(19) United States
(12) Patent Application Publication
(10) Pub. No.: US 2014/0363561 A1
(43) Pub. Date: Dec. 11, 2014

(54) TAL-MEDIATED TRANSFER DNA INSERTION

(71) Applicant: J.R. SIMPLOT COMPANY, BOISE, ID (US)

(72) Inventors: CAIUS M. ROMMENS, BOISE, ID (US); HUI DUAN, BOISE, ID (US); J. TROY WEEKS, BOISE, ID (US)

(73) Assignee: J.R. SIMPLOT COMPANY, BOISE, ID (US)

(21) Appl. No.: 14/212,520

(22) Filed: Mar. 14, 2014

Related U.S. Application Data
(60) Provisional application No. 61/790,434, filed on Mar. 15, 2013.

(51) Int. Cl. C12N 15/82; A23L 1/217; C12N 9/22

(52) U.S. Cl. CPC C12N 15/8213; C12N 9/22; A23L 1/217
USPC 426/637; 435/199; 435/320.1; 800/278; 800/298; 800/301

(57) ABSTRACT

The invention relates to methods for stably integrating a desired polynucleotide into a plant genome.

### CROSS-REFERENCE TO RELATED APPLICATIONS

This application claims the priority benefits of U.S. provisional patent 61/790,434 filed Mar. 15, 2013, the entire contents of which are incorporated herein by reference.

### FIELD OF THE INVENTION

The present invention relates to the field of plant biotechnology and provides methods for targeted transfer DNA insertion for the production of plants and plant products with desirable traits.

### BACKGROUND OF THE INVENTION

A plant can be modified through insertion of a DNA segment into its genome. The added DNA comprises genetic elements rearranged to produce RNA that either encodes a protein or triggers the degradation of specific native RNA. The prior art teaches a variety of sub-optimal methods that result in non-targeted (unpredictable and random) insertion.

There is a need in the art for an efficient and reproducible production of genetically engineered plants and plant products with desirable traits. The challenges associated with the employment of transgenic traits are disconcerting, especially because important quality issues have not effectively been addressed through conventional breeding.

### SUMMARY OF THE INVENTION

One aspect of the present invention is a method for stably integrating a desired polynucleotide into a plant genome, comprising:
(A) transforming plant material with a first vector comprising nucleotide sequences encoding TAL proteins designed to recognize a target sequence;
(B) transforming the plant material with a second vector comprising (i) a marker gene that is not operably linked to a promoter (“promoter-free marker cassette”) and which comprises a sequence homologous to the target sequence; and (ii) a desired polynucleotide; and
(C) identifying transformed plant material in which the desired polynucleotide is stably integrated.

In one embodiment, the transformed plant material is exposed to conditions that reflect the presence or absence of the marker gene in the transformed plant. In another embodiment, the marker gene is a herbicide resistance gene and the transformed plant material is exposed to herbicide. In one embodiment, the herbicide resistance gene is the ALS gene. In another embodiment, the promoter-free marker cassette is stably integrated into the plant genome.

In another embodiment, the invention provides a method for the targeted insertion of exogenous DNA into a plant comprising the steps of (i) transforming isolated plant cells with (A) a first binary vector comprising a promoter-less cassette comprising (a) a right border sequence linked to (b) a partial sequence of the Ubi7 intron 5′-untranslated region; (c) an Ubi7 monomer-encoding sequence fused to a mutated acetolactate synthase (ALS) gene; (d) a desired nucleotide sequence; and (e) a terminator sequence, wherein the desired nucleotide sequence is not operably linked to a promoter; and (B) a second binary vector comprising (a) a right border; (b) a forward expression cassette and a reverse expression cassette, each comprising a modified TAL effector operably linked to a strong constitutive promoter, and a terminator sequence; and (c) a sequence encoding an enzyme involved in cytokinin production, such as isopentenyl transferase (ipt), wherein the modified TAL effector is designed to bind the desired nucleotide sequence within an intron of potato's ubiquitin-7 (Ubi7) gene; and (ii) culturing the isolated plant cells under conditions that promote growth of plants that express the desired nucleotide sequence; wherein no vector backbone DNA is permanently inserted into the plant genome.

In a preferred aspect of the invention, the modified TAL effector comprises (a) a truncated C-terminal activation domain comprising a Fok1 endonuclease catalytic domain; (b) a codon-optimized target sequence binding domain comprising 16.5 repeat variable diresidues corresponding to the Ubi7 5′-untranslated intron sequence; and (c) an N-terminal region comprising a SV40 nuclear localization sequence.

In an additional preferred aspect of the invention, the desired nucleotide sequence is a silencing cassette targeting one or more genes selected from the group consisting of asparagine synthase 1 (Asn1), polyphenol oxidase (Ppo), and vacuolar invertase (Inv) genes. In an even more preferred aspect of the invention, the first binary vector further comprises a late blight resistance gene Vnt1 operably linked to its native promoter and terminator sequences.

In a different embodiment, the invention provides a transformed plant comprising in its genome an endogenous Ubi7 promoter operably linked to a desired exogenous nucleotide sequence operably linked to an exogenous terminator sequence. In one aspect of the invention, the expression of one or more genes selected from the group consisting of asparagine synthase 1 (Asn1), polyphenol oxidase (Ppo), and vacuolar invertase (Inv) genes in the transformed plant is down-regulated. In a preferred aspect of the invention, the plant further expresses a late blight resistance gene Vnt1.

In one embodiment, the transformed plant is a tuber-bearing plant. In a preferred embodiment, the tuber-bearing plant is a potato plant. Preferably, the transformed plant has a phenotype characterized by one or more of late blight resistance, black spot bruise tolerance, reduced cold-induced sweetening and reduced asparagine levels in its tubers.

In yet another embodiment, the invention provides a heat-processed product of the transformed plant of the invention. Preferably, the heat-processed product is a French fry, chip, crisp, potato, dehydrated potato or baked potato. In a preferred aspect of the invention, the heat-processed product has a lower level of acrylamide than a heat-processed product of a non-transformed plant of the same species.

In a different embodiment, the invention provides a modified TAL effector designed to bind to a desired sequence comprising (a) a truncated C-terminal activation domain comprising a catalytic domain; (b) a codon-optimized target sequence binding domain; and (c) an N-terminal region comprising a nuclear localization sequence. In a preferred aspect of the invention, the modified TAL effector is designed to bind the desired sequence within an intron of potato's ubiquitin-7 (Ubi7) gene. As such, the modified TAL effector comprises (a) a catalytic domain in the C-terminal activation domain comprising a Fok1 endonuclease; (b) a target sequence binding domain comprising 16.5 repeat variable diresidues corresponding to the Ubi7 5′-untranslated intron sequence; and (c) a SV40 nuclear localization sequence in the N-terminal region.

In yet another embodiment, the invention provides a binary vector comprising (a) a right border; (b) a forward expression cassette and a reverse expression cassette, each comprising a modified TAL effector according to claim 16 operably linked to a strong constitutive promoter and a terminator sequence; and (c) a sequence encoding an enzyme involved in cytokinine production, such as isopentenyl transferase (ipt).

In yet another embodiment, the invention provides a DNA construct comprising a promoter-less cassette comprising (a) a right border sequence linked to (b) a partial Ubi7 5′-untranslated intron sequence; (c) an Ubi7 monomer-encoding sequence fused to a mutated acetolactate synthase (ALS) gene; (d) a desired nucleotide sequence; (e) a terminator sequence; and (f) a left border, wherein the desired nucleotide sequence is not operably linked to a promoter. In a preferred aspect of the invention, the desired nucleotide sequence in the DNA construct is a silencing cassette targeting one or more genes selected from the group consisting of asparagine synthase 1 (Asn1), polyphenol oxidase (Ppo), and vacuolar invertase (Inv) genes. In an even more preferred aspect, the DNA construct further comprises a late blight resistance gene Vnt1 operably linked to its native promoter and terminator sequences.

In a different embodiment, the invention provides a kit for targeted insertion of exogenous DNA into a plant comprising: (A) a first binary vector comprising a promoter-less cassette comprising (a) a right border sequence linked to (b) a partial sequence of the Ubi7 intron 5′-untranslated region; (c) an Ubi7 monomer-encoding sequence fused to a mutated acetolactate synthase (ALS) gene; (d) a desired nucleotide sequence; and (e) a terminator sequence, wherein the desired nucleotide sequence is not operably linked to a promoter; and (B) a second binary vector comprising (a) a right border; (b) a forward expression cassette and a reverse expression cassette, each comprising a modified TAL effector operably linked to a strong constitutive promoter, and a terminator sequence; and (c) a sequence encoding isopentenyl transferase (ipt). In a preferred aspect of the invention, the modified TAL effector is designed to bind the desired nucleotide sequence within an intron of potato's ubiquitin-7 (Ubi7) gene, and comprises (a) a truncated C-terminal activation domain comprising a Fok1 endonuclease catalytic domain; (b) a codon-optimized target sequence binding domain comprising 16.5 repeat variable diresidues corresponding to the Ubi7 5′-untranslated intron sequence; and (c) an N-terminal region comprising a SV40 nuclear localization sequence. In another preferred aspect of the invention, the desired nucleotide sequence is a silencing cassette targeting one or more genes selected from the group consisting of asparagine synthase 1 (Asn1), polyphenol oxidase (Ppo), and vacuolar invertase (Inv) genes. In an even more preferred aspect of the invention, the first binary vector further comprises a late blight resistance gene Vnt1 operably linked to its native promoter and terminator sequences.

The foregoing general description and the following detailed description are exemplary and explanatory and are intended to provide further explanation of the invention as claimed. Other objects, advantages and novel features will be readily apparent to those skilled in the art from the following detailed description of the invention.

### BRIEF DESCRIPTION OF THE DRAWINGS

This application contains at least one drawing executed in color.

FIG. 1 illustrates the transfer DNA organization of the plasmid pSIM2168. Sequence shown in bottom panel starts from the 25 bp unhighlighted right border. The light gray highlighted sequence is part of the Ubi7 intron, the dark gray highlighted sequence is the Ubi7 monomer and the remaining unhighlighted sequence is part of the potato ALS gene coding.

FIG. 2 illustrates the forward (E3) and reverse (E4) TAL effector cassettes in the vector pSIM2170.

FIG. 3 shows the right border testing cassette described in Example 9.

FIG. 4 illustrates the DNA organization of the plasmid pSIM2162 carrying the Ubi7::ALS cassette.

FIG. 5 shows the organization of the forward (5A) and reverse (5B) effector proteins.

FIG. 6 illustrates the organization of the plasmid pSIM216 carrying the target sequence containing the forward and reverse recognition sites positioned immediately downstream from the start codon of the GUS reporter gene.

FIG. 7 shows GUS staining of Nicothiana benthamiana leaves following Agrobacterium infiltration. Left panel: infiltration with target vector pSIM2167 alone. Right panel; infiltration with target vector pSIM2167 and TAL effector vector pSIM2170.

FIG. 8 shows the sequence of PCR-amplified target region of the plasmid pSIM2167 after co-infiltration with the TAL effector vector pSIM2170. Effector recognition site is gray highlighted. Modifications on target sequence are small deletion (majority form) and substitutions (dark gray highlighted).

FIG. 9 shows the sequences of fragments from targeted insertion-specific PCR. The first non-highlighted sequence and the first light gray highlighted sequence are potato genome sequences. non highlighted sequence: part of Uni7-like promoter; light gray highlighted sequence: Uni7-like intron. The remaining sequences are from the pSIM2168 vector. Dark gray sequence: part of Ubi7 intron; non-highlighted sequence: Ubi7 monomer; light gray highlighted sequence: part of the ALS coding sequence.

FIG. 10 shows inter-node explants grown in hormone-free medium containing timentin and 0.0 mg/l imazamox (left panel) or 2.0 mg/l imazamox (right panel). No fully developed normal shoots were visible when the inter-node explants were grown in a medium containing imazamox.

FIG. 11 shows Ranger Russet control (RR-C) lines and herbicide-resistant Ranger Russet lines co-transformed with the pSIM2170 and pSIM2168 plasmids for targeted insertion, challenged with P. infestans late blight strain US8 BF6 for the development of disease symptoms, at seven days after infection.

FIG. 12 depicts the southern blot gels for selected herbicide-resistant Ranger Russet lines co-transformed with the pSIM2170 and pSIM2168 plasmids for targeted insertion. Left panel: invertase probe; right panel: Vnt1 promoter probe. Each additional band in the transformed lines, as compared to the Ranger Russet control (RR) lines, indicates a single copy transgene for lines RR-36 (36) and RR-39 (39). Transformed lines RR-26 and RR-32 are not shown.

### DETAILED DESCRIPTION OF THE INVENTION

One aspect of the present invention is the transient expression of transcription activator-like effector proteins designed to bind to, and consequently cut, a desired genomic target locus, thereby facilitating the insertion of a desired polynucleotide at that particular target locus. Accordingly, the present invention encompasses the transformation of plant material with a vector that contains an expression cassette encoding peptides or proteins that form appropriate TAL dimers that recognize and cleave a target locus, and a second vector that comprises one or more desired expression cassettes. Such desired expression cassettes may encode a particular protein or gene silencing transcript. In one embodiment, the second vector may comprise a cassette referred to herein as a “promoter-free” cassette, which comprises (i) a marker gene or gene that encodes a desired phenotype, and appropriate other regulatory elements that would facilitate appropriate expression of that marker gene if it was operably linked to a promoter; and (ii) a nucleotide region homologous to the endogenous target locus site destination. Thus, in one embodiment, the second vector comprises at least (i) an expression cassette encoding a desired polynucleotide (such as one that encodes a protein or untranslatable RNA transcript), and (ii) a promoter-free marker cassette that comprises a marker gene operably linked to a regulatory element such as a terminator or 3-untranslated region, along with the homologous target site region.

The promoter-free marker cassette and the expression cassette(s) of the second vector ideally travel together so that both become integrated into the target locus as a consequence of TAL-mediated activity (brought about by the other, TAL-encoding, vector). Ideally, the promoter-free cassette and the expression cassette(s) are integrated into a desired site at the target locus suitably near, e.g., downstream or upstream as the case may be, of one or more functional endogenous promoters or endogenous regulatory elements, such that the endogenous promoter or regulatory element expresses the marker gene of the promoter-free marker cassette. The appropriate design of the TAL sequences to recognize such a target sequence downstream of an endogenous gene promoter or regulatory element that initiates gene expression, such as an enhancer element, is therefore important in helping to ensure that the expression cassette(s) and promoter-free marker cassette are integrated at a particular genomic location time and again between different transformation events.

The marker gene is important because if the promoter-free marker cassette is appropriately integrated, the marker gene will be expressed by the endogenous regulatory element, and depending on the type of marker will (a) effectively identify successful transformants, and (b) give a preliminary indication of the successful insertion of the co joined expression cassette(s) at the desired target location. Thus, if the marker gene is a herbicide resistant gene, the transformed plant cells may be cultured on the relevant herbicide and cells that survive reflect those that are transformed with the herbicide resistance gene at the desired target locus near a functional endogenous promoter.

Thus, the ability to routinely insert an expression cassette at the same genomic locus between different transformation events is highly desirable and advantageous and cost-effective because this reduces the magnitude of screens needed to identify integration events that would otherwise occur randomly in different genomic environments. Those differences in integration loci can often disrupt the local genomic environment detrimentally, knock-out essential genes, or place the desired expression cassettes in loci that fail to express the integrated DNA.

Accordingly, the homologous target site region present in the promoter-free marker cassette is specifically designed to match up with the endogenous target site sequence that the TAL protein dimer of the other vector is also designed to recognize, bind to, and cut. Thus, both the promoter-free marker cassette and the TAL expression cassette contain sequences unique to the endogenous genomic target locus, such that the promoter-free marker cassette and its co-joined desired expression cassettes, is inserted into the precise target locus site cut by the TALs.

The present invention is not limited to the insertion of promoter-free marker cassettes and expression cassettes into a genomic locus or nearby an endogenous promoter or regulatory element. Rather, the present invention encompasses the use of the inventive method to stack cassettes in a modular fashion based upon the design of TAL sequences and homologous regions that recognize polynucleotide sequences from prior transformation events. That is, in one embodiment, a plant may have already been stably transformed with Expression Cassette A that expresses Gene X at a particular or random site in the plant genome. The present TAL-mediated integration method allows for the design of TAL sequences that recognize a sequence perhaps downstream of Gene X in Expression Cassette A, such that the TAL dimer effectively cleaves the plant genome at that Gene X site. If the promoter-free marker cassette—or any expression cassette—comprises a homologous region to that Gene X site, then it is possible to introduce that cassette immediately downstream of Gene X. As mentioned, it is not necessary that in all cases the present invention must utilize a promoter-free marker system for it may be the case that the gene-of-interest integrated downstream of the pre-transformed Gene X plant produces a detectable and desired trait in and of itself. Furthermore, the additional expression cassette may contain its own promoter or may be promoter-free such that the gene-of-interest is expressed from the promoter or regulatory element of Expression Cassette A.

Accordingly, in one embodiment, the present invention encompasses the de novo insertion of a desired expression cassette into a target locus using the promoter-free marker design to identify successful and appropriate transformants. In another embodiment, the present invention encompasses the subsequent insertion of one or more additional expression cassettes, which may or may not include a promoter-free marker cassette, downstream or upstream of a prior integration event. Thus, in the latter approach, the present invention permits the ability to stack genes at precise and defined locations within a plant genome by effectively linking together different expression cassettes even though this is done via different transformations.

In one embodiment, it is desirable to only transiently express the TAL proteins such that the only DNA that becomes stably integrated into the plant genome belongs to the desired expression cassette(s) and promoter-free marker cassette, if used.

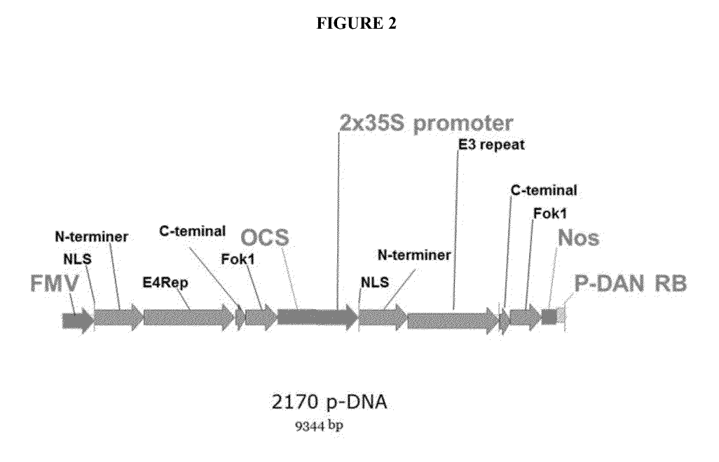

Accordingly, one aspect of the present invention encompasses (1) identifying in the genome of a plant a desired target locus sequence; (2) designing corresponding TAL sequences that specifically recognize that target locus sequence; and, optionally, (3) assaying the designed TAL sequences in an infiltration assay, for instance, to test if the corresponding TAL dimer, when formed, cuts appropriately. TALs that work can then be subcloned into a transient expression transformation vector, such as shown in FIG. 2. Such steps are described in detail herein. See for instance Examples 10 and 11.

A second vector can then be designed comprising one or more desired expression cassettes along with the promoter-free marker cassette, and both the TAL vector and the second vector subsequently transformed into one or two strains of Agrobacteria.

Plant material, such as explants, calli, cells, leaves, or stems, can then be transformed using these Agrobacteria. In one embodiment, the transformed plant material can be grown into calli on media that does not contain any selection component. That is, for the ease of illustration, if the second vector comprises a herbicide resistance marker gene that is not operably linked to a promoter, then the transformed plant material would initially be cultured on media that does not contain herbicide for a certain period of time. After that period of time, the plant material may be placed on callus induction media that does contain herbicide. Those materials that survive can then be placed on shoot induction media that also contains the same herbicide until shoots develop and survive for a period of time. The shoots that grow on herbicide media are therefore likely to contain the stably integrated herbicide resistance gene in their genomes along with the actual desired expression cassettes. Those herbicide-resistant shoots or leaves that grow from those shoots can then be subjected to PCR and other molecular analyses to determine if they contain the marker and also the desired expression cassette(s) in the correct and expected genomic target location. This method is described herein in detail, see for instance Example 4. When the ALS gene was used as the marker gene in the promoter-free arrangement, as discussed herein, 80% of the analyzed transformed shoots/leaves contained the desired insert stably integrated in the desired genomic locus.

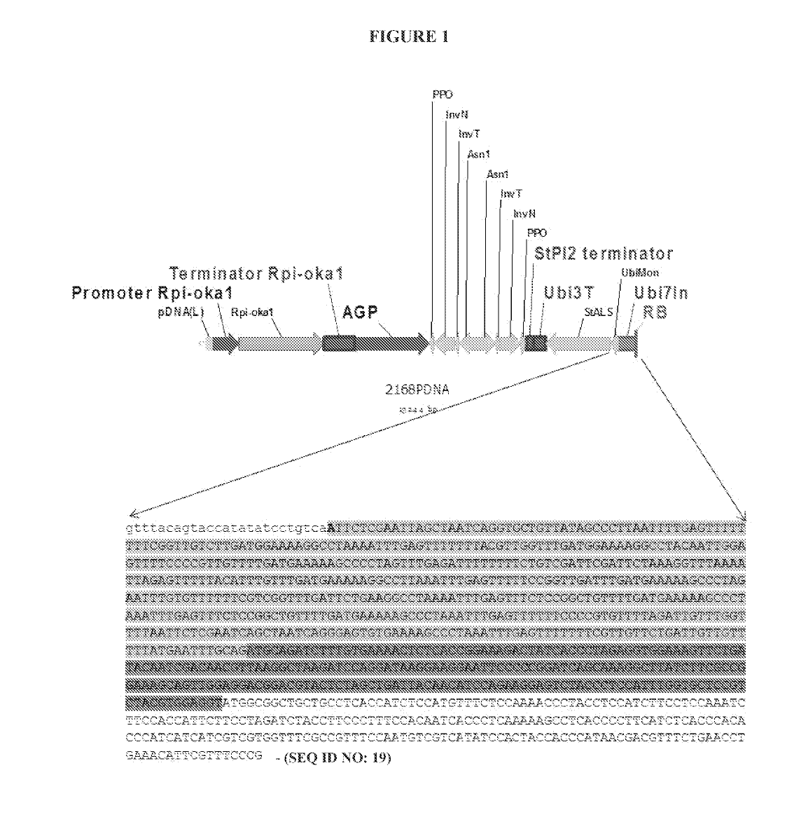

In one embodiment, the second vector comprises the gene expression cassette for late blight and a gene silencing cassette for silencing PPO, ASN1, and invertase, in addition to a promoter-free ALS herbicide marker gene, as shown in pSIM2168 (FIG. 1). In this case, the ALS gene is not operably linked to a promoter but it is operably linked to a terminator and includes, upstream, a sequence homologous to a region of the endogenous plant Ubi7 gene intron and part of the Ubi7 exon #1. FIG. 2 depicts the corresponding vector (pSIM2170) that expresses the E4Rep and E3 repeat TAL sequences that are also designed to recognize a naturally-occurring sequence within the Ubi7 gene intron. Both pSIM2168 and pSIM2170 are transformed into potato stem explants and subjected to the method described above and as described in methodological detail in the Examples provided herein. The results show that the inventive approach was successful in using TAL-mediated integration to stably integrate the cassettes of pSIM2168 into the precise target location desired in the endogenous Ubi7 gene intron.

The present inventive methods are not limited to the introduction of such vectors using transfer-DNAs or Agrobacterium. It is possible to use particle bombardment, for instance, without any Agrobacterium or T-DNA components. In one embodiment, for instance, it is possible to coat particle bombardment particles with DNA encoding the desired expression cassette(s) and promoter-free marker cassette, and also coat the same particles with TAL proteins or TAL protein dimers. In this fashion therefore a particle may comprise both encoding DNA and TAL proteins, or some particles may be coated with either the encoding DNA or the TAL proteins. In any event, plant material can be bombarded with such coated particles whereupon when the particles enter the plant cell, the TAL proteins function as intended to cut the genomic sequence at a desired site and integrate the co-delivered DNA. See for instance Martin-Ortigosa et al., Adv. Funct. Mater. 22, 3576-3582 (2012), which is incorporated herein by reference, for examples of how to use particle bombardment to co-deliver proteins and DNA into plants.

As used herein, a “desired polynucleotide” is essentially any polynucleotide or series of DNA sequences within an expression cassette or gene silencing cassette that the user desires to be integrated into the plant genome. Accordingly, “desired polynucleotide” may be used interchangeably with “cassette” “expression cassette” or “silencing cassette” herein. A desired polynucleotide in any expression cassette can be operably linked to any kind or strength of promoter and its expression is not necessarily therefore dependent on the expression of an endogenous plant genomic promoter.

While it is desirable to stably transform plant genomes according to the present TAL-mediated integration technology, another embodiment of the inventive methods encompasses the integration of a desired polynucleotide into any form or sample of nucleic acid, not

TALs

Transcription activator-like (TAL) effectors are plant pathogenic bacterial proteins that contain modular DNA binding domains that facilitate site-specific integration of DNAs into a particularly desired target site, such as in a plant genome. These domains comprise tandem, polymorphic amino acid repeats that individually specify contiguous nucleotides in DNA that are useful for directing the targeted site-specific integration approach.

A central repeat domain may comprise between 1.5 and 33.5 repeats typically 34 residues in length. An example of a repeat sequence is:

LTPEQVVAIASHDGGKQALETVQRLLPVLCQAHG

The residues at the 12th and 13th positions can be hypervariable known as the “repeat variable diresidue” or RVD. There is a relationship between the identity of these two residues in sequential repeats and sequential DNA bases in the TAL effector's target site. The code between RVD sequence and target DNA base can be expressed as:

NI=A

HD=C

NG=T

NN=R (G or A), and

NS=N (A, C, G, or T).

RVD NK can target G, but TAL effector nucleases (TALENs) that exclusively use NK instead of NN to target G can be less active

Target sites of TAL effectors may include a T flanking the 5′-base targeted by the first repeat perhaps due to a contact between this T nucleotide and a conserved tryptophan in the region N-terminal of the central repeat domain.

See also the following publications which are all incorporated herein by reference in their entirety: Boch J, Bonas U (September 2010). “XanthomonasAvrBs3 Family-Type III Effectors: Discovery and Function”. Annual Review of Phytopathology 48: 419-36; Voytas D F, Joung J K (December 2009). “Plant science. DNA binding made easy”. Science 326 (5959): 1491-2. Bibcode 2009; Moscou M J, Bogdanove A J (December 2009). “A simple cipher governs DNA recognition by TAL effectors”. Science 326 (5959): 1501; Boch J, Scholze H, Schornack S et al. (December 2009). “Breaking the code of DNA binding specificity of TAL-type III effectors”. Science 326 (5959): 1509-12; Morbitzer, R.; Romer, P.; Boch, J.; Lahaye, T. (2010). “Regulation of selected genome loci using de novo-engineered transcription activator-like effector (TALE)-type transcription factors”. Proceedings of the National Academy of Sciences 107 (50): 21617-21622; Miller, J. C.; Tan, S.; Qiao, G.; Barlow, K. A.; Wang, J.; Xia, D. F.; Meng, X.; Paschon, D. E. et al. (2010). “A TALE nuclease architecture for efficient genome editing”. Nature Biotechnology 29 (2): 14; Huang, P.; Xiao, A.; Zhou, M.; Zhu, Z.; Lin, S.; Zhang, B. (2011). “Heritable gene targeting in zebrafish using customized TALENs”. Nature Biotechnology 29 (8): 699; and Mak, A. N.-S.; Bradley, P.; Cernadas, R. A.; Bogdanove, A. J.; Stoddard, B. L. (2012). “The Crystal Structure of TAL Effector PthXo1 Bound to Its DNA Target”. Science. doi:10.1126/science.1216211.

Markers

Examples of the categories of marker genes that can be used as disclosed herein in the promoter-free marker gene cassette include, but are not limited to, herbicide tolerance, pesticide tolerance insect resistance, tolerance to stress, enhanced flavor or stability of the fruit or seed, or the ability to synthesize useful, non-plant proteins, e.g., medically valuable proteins or the ability to generate altered concentrations of plant proteins, and related impacts on the plant, e.g., altered levels of plant proteins catalyzing production of plant metabolites including secondary plant metabolites.

This invention provides methods and kits for the targeted insertion of desired nucleotide sequences into plants, by inserting promoter-less desired nucleotide sequences into the intron sequence of the ubiquitin-7 (Ubi7) gene, such that the expression of exogenous nucleotide sequences in the plants is driven by the endogenous Ubi7 promoter,. In particular, the inventors were able to create binary vectors for the transient expression of transcription activator-like effector nucleases specifically designed to bind desired nucleotide sequences within the intron sequence of the Ubi7 gene, such that when these vectors are introduced into plant cells together with binary vectors carrying the targeted promoter-less nucleotide sequences, the desired nucleotide sequences are inserted into the intron sequence of the Ubi7 gene with proper orientation and spacing, and their expression is driven by the endogenous Ubi7 promoter. The transformed plants regenerating from the transformed explants thus obtained carry only the targeted sequences.

The invention further provides plants transformed by the methods of the invention, as well as the binary vectors for transient and permanent transformation.

The technology strategy of the present invention addresses the need to efficiently produce genetically engineered plants and plant products with desirable traits by targeted transformation, such that the nutritional value and agronomic characteristics of plant crops, and in particular tuber-bearing plants, such as potato plants, may be improved. Desirable traits include, but are not limited to, high tolerance to impact-induced black spot bruise, reduced formation of the acrylamide precursor asparagine, reduced accumulation of reducing sugars and reduced accumulation of toxic Maillard products, including acrylamide. Thus, the present invention allows the targeted insertion of these desirable traits into a plant genome by reducing the expression of enzymes, such as polyphenol oxidase (PPO), which is responsible for black spot bruise, and asparagine synthetase-1 (Asn-1), which is responsible for the accumulation of asparagine, a precursor in acrylamide formation, and by increasing the expression of the late blight resistance gene Vnt1.

The present invention uses terms and phrases that are well known to those practicing the art. Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry and hybridization described herein are those well known and commonly employed in the art. Standard techniques are used for recombinant nucleic acid methods, polynucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. The techniques and procedures are generally performed according to conventional methodology (Molecular Cloning, A Laboratory Manual, 3rd. edition, edited by Sambrook & Russel Cold Spring Harbor Laboratory Press, Cold Spring Harbor, N.Y., 2001).

Agrobacterium or bacterial transformation: as is well known in the field, Agrobacteria that are used for transforming plant cells are disarmed and virulent derivatives of, usually, Agrobacterium tumefaciens or Agrobacterium rhizogenes. Upon infection of plants, explants, cells, or protoplasts, a single Agrobacterium strain containing a binary vector comprising a TAL effector cassette and a binary vector comprising the gene of interest, or two separate Agrobacterium strains, one containing a binary vector comprising a TAL effector cassette, and the other containing a binary vector comprising the gene of interest, transfer a desired DNA segment from a plasmid vector to the plant cell nucleus. The vector typically contains a desired polynucleotide that is located between the borders of a T-DNA. However, any bacteria capable of transforming a plant cell may be used, such as, Rhizobium trifolii, Rhizobium leguminosarum, Phyllobacterium myrsinacearum, SinoRhizobium meliloti, and MesoRhizobium loti. The present invention is not limited to the use of bacterial transformation systems. Any organism however that contains the appropriate cellular machinery and proteins to accomplish plant cell transformation.

Angiosperm: vascular plants having seeds enclosed in an ovary. Angiosperms are seed plants that produce flowers that bear fruits. Angiosperms are divided into dicotyledonous and monocotyledonous plant.

Antibiotic Resistance: ability of a cell to survive in the presence of an antibiotic. Antibiotic resistance, as used herein, results from the expression of an antibiotic resistance gene in a host cell. A cell may have resistance to any antibiotic. Examples of commonly used antibiotics include kanamycin and hygromycin.

Dicotyledonous plant (dicot): a flowering plant whose embryos have two seed halves or cotyledons, branching leaf veins, and flower parts in multiples of four or five. Examples of dicots include but are not limited to, potato, sugar beet, broccoli, cassava, sweet potato, pepper, poinsettia, bean, alfalfa, soybean, and avocado.

Endogenous: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species.

Expression cassette: polynucleotide that may comprise, from 5′ to 3′, (a) a first promoter, (b) a sequence comprising (i) at least one copy of a gene or gene fragment, or (ii) at least one copy of a fragment of the promoter of a gene, and (c) either a terminator or a second promoter that is positioned in the opposite orientation as the first promoter.

Foreign: “foreign,” with respect to a nucleic acid, means that that nucleic acid is derived from non-plant organisms, or derived from a plant that is not the same species as the plant to be transformed or is not derived from a plant that is not interfertile with the plant to be transformed, does not belong to the species of the target plant. According to the present invention, foreign DNA or RNA represents nucleic acids that are naturally occurring in the genetic makeup of fungi, bacteria, viruses, mammals, fish or birds, but are not naturally occurring in the plant that is to be transformed. Thus, a foreign nucleic acid is one that encodes, for instance, a polypeptide that is not naturally produced by the transformed plant. A foreign nucleic acid does not have to encode a protein product.

Gene: A gene is a segment of a DNA molecule that contains all the information required for synthesis of a product, polypeptide chain or RNA molecule that includes both coding and non-coding sequences. A gene can also represent multiple sequences, each of which may be expressed independently, and may encode slightly different proteins that display the same functional activity. For instance, the asparagine synthetase 1 and 2 genes can, together, be referred to as a gene.

Genetic element: a “genetic element” is any discreet nucleotide sequence such as, but not limited to, a promoter, gene, terminator, intron, enhancer, spacer, 5′-untranslated region, 3′-untranslated region, or recombinase recognition site.

Genetic modification: stable introduction of DNA into the genome of certain organisms by applying methods in molecular and cell biology.

Gymnosperm: as used herein, refers to a seed plant that bears seed without ovaries. Examples of gymnosperms include conifers, cycads, ginkgos, and ephedras.

Introduction: as used herein, refers to the insertion of a nucleic acid sequence into a cell, by methods including infection, transfection, transformation or transduction.

Monocotyledonous plant (monocot): a flowering plant having embryos with one cotyledon or seed leaf, parallel leaf veins, and flower parts in multiples of three. Examples of monocots include, but are not limited to maize, rice, oat, wheat, barley, and sorghum.

Native: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species.

Native DNA: any nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species. In other words, a native genetic element represents all genetic material that is accessible to plant breeders for the improvement of plants through classical plant breeding. Any variants of a native nucleic acid also are considered “native” in accordance with the present invention. For instance, a native DNA may comprise a point mutation since such point mutations occur naturally. It is also possible to link two different native DNAs by employing restriction sites because such sites are ubiquitous in plant genomes.

Native Nucleic Acid Construct: a polynucleotide comprising at least one native DNA.

Operably linked: combining two or more molecules in such a fashion that in combination they function properly in a plant cell. For instance, a promoter is operably linked to a structural gene when the promoter controls transcription of the structural gene.

Overexpression: expression of a gene to levels that are higher than those in control plants.

P-DNA: a plant-derived transfer-DNA (“P-DNA”) border sequence of the present invention is not identical in nucleotide sequence to any known bacterium-derived T-DNA border sequence, but it functions for essentially the same purpose. That is, the P-DNA can be used to transfer and integrate one polynucleotide into another. A P-DNA can be inserted into a tumor-inducing plasmid, such as a Ti-plasmid from Agrobacterium in place of a conventional T-DNA, and maintained in a bacterium strain, just like conventional transformation plasmids. The P-DNA can be manipulated so as to contain a desired polynucleotide, which is destined for integration into a plant genome via bacteria-mediated plant transformation. The P-DNA comprises at least one border sequence. See Rommens et al. 2005 Plant Physiology 139: 1338-1349, which is incorporated herein by reference. In certain embodiments of the invention, the T-DNA is replaced by the P-DNA.

Phenotype: phenotype is a distinguishing feature or characteristic of a plant, which may be altered according to the present invention by integrating one or more “desired polynucleotides” and/or screenable/selectable markers into the genome of at least one plant cell of a transformed plant. The “desired polynucleotide(s)” and/or markers may confer a change in the phenotype of a transformed plant, by modifying any one of a number of genetic, molecular, biochemical, physiological, morphological, or agronomic characteristics or properties of the transformed plant cell or plant as a whole.

Plant tissue: a “plant” is any of various photosynthetic, eukaryotic, multicellular organisms of the kingdom Plantae characteristically producing embryos, containing chloroplasts, and having cellulose cell walls. A part of a plant, i.e., a “plant tissue” may be treated according to the methods of the present invention to produce a transgenic plant. Many suitable plant tissues can be transformed according to the present invention and include, but are not limited to, somatic embryos, pollen, leaves, stems, calli, stolons, microtubers, and shoots. Thus, the present invention envisions the transformation of angiosperm and gymnosperm plants such as wheat, maize, rice, barley, oat, sugar beet, potato, tomato, alfalfa, cassava, sweet potato, and soybean. According to the present invention “plant tissue” also encompasses plant cells. Plant cells include suspension cultures, callus, embryos, meristematic regions, callus tissue, leaves, roots, shoots, gametophytes, sporophytes, pollen, seeds and microspores. Plant tissues may be at various stages of maturity and may be grown in liquid or solid culture, or in soil or suitable media in pots, greenhouses or fields. A plant tissue also refers to any clone of such a plant, seed, progeny, propagule whether generated sexually or asexually, and descendents of any of these, such as cuttings or seed. Of particular interest are potato, maize, and wheat.

Plant transformation and cell culture: broadly refers to the process by which plant cells are genetically modified and transferred to an appropriate plant culture medium for maintenance, further growth, and/or further development. Such methods are well known to the skilled artisan.

Processing: the process of producing a food from (1) the seed of, for instance, wheat, corn, coffee plant, or cocoa tree, (2) the tuber of, for instance, potato, or (3) the root of, for instance, sweet potato and yam comprising heating to at least 120° C. Examples of processed foods include bread, breakfast cereal, pies, cakes, toast, biscuits, cookies, pizza, pretzels, tortilla, French fries, oven-baked fries, potato chips, hash browns, roasted coffee, and cocoa.

Progeny: a “progeny” of the present invention, such as the progeny of a transgenic plant, is one that is born of, begotten by, or derived from a plant or the transgenic plant. Thus, a “progeny” plant, i.e., an “F1” generation plant is an offspring or a descendant of the transgenic plant produced by the inventive methods. A progeny of a transgenic plant may contain in at least one, some, or all of its cell genomes, the desired polynucleotide that was integrated into a cell of the parent transgenic plant by the methods described herein. Thus, the desired polynucleotide is “transmitted” or “inherited” by the progeny plant. The desired polynucleotide that is so inherited in the progeny plant may reside within a T-DNA or P-DNA construct, which also is inherited by the progeny plant from its parent. The term “progeny” as used herein, also may be considered to be the offspring or descendants of a group of plants.

Promoter: promoter is intended to mean a nucleic acid, preferably DNA that binds RNA polymerase and/or other transcription regulatory elements. A promoter is a nucleic acid sequence that enables a gene with which it is associated to be transcribed. A regulatory region refers to nucleic acid sequences that influence and/or promote initiation of transcription. Promoters are typically considered to include regulatory regions, such as enhancer or inducer elements.

Eukaryotic promoters typically lie upstream of the gene to which they are most immediately associated. Promoters can have regulatory elements located several kilobases away from their transcriptional start site, although certain tertiary structural formations by the transcriptional complex can cause DNA to fold, which brings those regulatory elements closer to the actual site of transcription. Many eukaryotic promoters contain a “TATA box” sequence, typically denoted by the nucleotide sequence, TATAAA. This element binds a TATA binding protein, which aids formation of the RNA polymerase transcriptional complex. The TATA box typically lies within 50 bases of the transcriptional start site.

Eukaryotic promoters also are characterized by the presence of certain regulatory sequences that bind transcription factors involved in the formation of the transcriptional complex. An example is the E-box denoted by the sequence CACGTG, which binds transcription factors in the basic-helix-loop-helix family. There also are regions that are high in GC nucleotide content.

A polynucleotide may be linked in two different orientations to the promoter. In one orientation, e.g., “sense”, at least the 5′-part of the resultant RNA transcript will share sequence identity with at least part of at least one target transcript. In the other orientation designated as “antisense”, at least the 5′-part of the predicted transcript will be identical or homologous to at least part of the inverse complement of at least one target transcript.

A plant promoter is a promoter capable of initiating transcription in plant cells whether or not its origin is a plant cell. Exemplary plant promoters include, but are not limited to, those that are obtained from plants, plant viruses, and bacteria such as Agrobacterium or Rhizobium which comprise genes expressed in plant cells. Examples of promoters under developmental control include promoters that preferentially initiate transcription in certain tissues, such as xylem, leaves, roots, or seeds. Such promoters are referred to as tissue-preferred promoters. Promoters which initiate transcription only in certain tissues are referred to as tissue-specific promoters. A cell type-specific promoter primarily drives expression in certain cell types in one or more organs, for example, vascular cells in roots or leaves. An inducible or repressible promoter is a promoter which is under environmental control. Examples of environmental conditions that may effect transcription by inducible promoters include anaerobic conditions or the presence of light. Tissue specific, tissue preferred, cell type specific, and inducible promoters constitute the class of non-constitutive promoters. A constitutive promoter is a promoter which is active under most environmental conditions, and in most plant parts.

Polynucleotide is a nucleotide sequence, comprising a gene coding sequence or a fragment thereof, (comprising at least 15 consecutive nucleotides, preferably at least 30 consecutive nucleotides, and more preferably at least 50 consecutive nucleotides), a promoter, an intron, an enhancer region, a polyadenylation site, a translation initiation site, 5′ or 3′ untranslated regions, a reporter gene, a selectable marker or the like. The polynucleotide may comprise single stranded or double stranded DNA or RNA. The polynucleotide may comprise modified bases or a modified backbone. The polynucleotide may be genomic, an RNA transcript (such as an mRNA) or a processed nucleotide sequence (such as a cDNA). The polynucleotide may comprise a sequence in either sense or antisense orientations.

An isolated polynucleotide is a polynucleotide sequence that is not in its native state, e.g., the polynucleotide is comprised of a nucleotide sequence not found in nature or the polynucleotide is separated from nucleotide sequences with which it typically is in proximity or is next to nucleotide sequences with which it typically is not in proximity.

Seed: a “seed” may be regarded as a ripened plant ovule containing an embryo, and a propagative part of a plant, as a tuber or spore. Seed may be incubated prior to Agrobacterium-mediated transformation, in the dark, for instance, to facilitate germination. Seed also may be sterilized prior to incubation, such as by brief treatment with bleach. The resultant seedling can then be exposed to a desired strain of Agrobacterium.

Selectable/screenable marker: a gene that, if expressed in plants or plant tissues, makes it possible to distinguish them from other plants or plant tissues that do not express that gene. Screening procedures may require assays for expression of proteins encoded by the screenable marker gene. Examples of selectable markers include herbicide resistance genes, such as acetolactate synthase (ALS), the neomycin phosphotransferase (NptII) gene encoding kanamycin and geneticin resistance, the hygromycin phosphotransferase (HptII) gene encoding resistance to hygromycin, or other similar genes known in the art.

Sensory characteristics: panels of professionally trained individuals can rate food products for sensory characteristics such as appearance, flavor, aroma, and texture. Thus, the present invention contemplates improving the sensory characteristics of a plant product obtained from a plant that has been modified according to the present invention to manipulate its tuber yield production.

Sequence identity: as used herein, “sequence identity” or “identity” in the context of two nucleic acid or polypeptide sequences includes reference to the residues in the two sequences which are the same when aligned for maximum correspondence over a specified region. A homologous region or sequence as used herein therefore describes a sequence that shares some degree of sequence identity with a target genomic loci. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11 17 (1988) e.g., as implemented in the program PC/GENE (Intelligenetics, Mountain View, Calif., USA).

As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Sequence identity” has an art-recognized meaning and can be calculated using published techniques. See COMPUTATIONAL MOLECULAR BIOLOGY, Lesk, ed. (Oxford University Press, 1988), BIOCOMPUTING: INFORMATICS AND GENOME PROJECTS, Smith, ed. (Academic Press, 1993), COMPUTER ANALYSIS OF SEQUENCE DATA, PART I, Griffin & Griffin, eds., (Humana Press, 1994), SEQUENCE ANALYSIS IN MOLECULAR BIOLOGY, Von Heinje ed., Academic Press (1987), SEQUENCE ANALYSIS PRIMER, Gribskov & Devereux, eds. (Macmillan Stockton Press, 1991), and Carillo & Lipton, SIAM J. Applied Math. 48: 1073 (1988). Methods commonly employed to determine identity or similarity between two sequences include but are not limited to those disclosed in GUIDE TO HUGE COMPUTERS, Bishop, ed., (Academic Press, 1994) and Carillo & Lipton, supra. Methods to determine identity and similarity are codified in computer programs. Preferred computer program methods to determine identity and similarity between two sequences include but are not limited to the GCG program package (Devereux et al., Nucleic Acids Research 12: 387 (1984)), BLASTP, BLASTN, FASTA (Atschul et al., J. Mol. Biol. 215: 403 (1990)), and FASTDB (Brutlag et al., Comp. App. Biosci. 6: 237 (1990)).

Silencing: The unidirectional and unperturbed transcription of either genes or gene fragments from promoter to terminator can trigger post-transcriptional silencing of target genes. Initial expression cassettes for post-transcriptional gene silencing in plants comprised a single gene fragment positioned in either the antisense (McCormick et al., U.S. Pat. No. 6,617,496; Shewmaker et al., U.S. Pat. No. 5,107,065) or sense (van der Krol et al., Plant Cell 2:291-299, 1990) orientation between regulatory sequences for transcript initiation and termination. In Arabidopsis, recognition of the resulting transcripts by RNA-dependent RNA polymerase leads to the production of double-stranded (ds) RNA. Cleavage of this dsRNA by Dicer-like (Dcl) proteins such as Dcl4 yields 21-nucleotide (nt) small interfering RNAs (siRNAs). These siRNAs complex with proteins including members of the Argonaute (Ago) family to produce RNA-induced silencing complexes (RISCs). The RISCs then target homologous RNAs for endonucleolytic cleavage.

More effective silencing constructs contain both a sense and antisense component, producing RNA molecules that fold back into hairpin structures (Waterhouse et al., Proc Natl Acad Sci USA 95: 13959-13964, 1998). The high dsRNA levels produced by expression of inverted repeat transgenes were hypothesized to promote the activity of multiple Dcls. Analyses of combinatorial Dcl knockouts in Arabidopsis supported this idea, and also identified Dcl4 as one of the proteins involved in RNA cleavage.

One component of conventional sense, antisense, and double-strand (ds) RNA-based gene silencing constructs is the transcriptional terminator. WO 2006/036739, which is incorporated in its entirety by reference, shows that this regulatory element becomes obsolete when gene fragments are positioned between two oppositely oriented and functionally active promoters. The resulting convergent transcription triggers gene silencing that is at least as effective as unidirectional ‘promoter-to-terminator’ transcription. In addition to short variably-sized and non-polyadenylated RNAs, terminator-free cassette produced rare longer transcripts that reach into the flanking promoter. Replacement of gene fragments by promoter-derived sequences further increased the extent of gene silencing.

TAL effectors (TALE) are proteins secreted by Xanthomonas bacteria characterized by the presence of a DNA binding domain that contains a repeated highly conserved 33-34 amino acid sequence, except for the highly variable 12th and 13th amino acids, which show a strong correlation with specific nucleotide recognition. These proteins can bind promoter sequences in the host plant and activate the expression of plant genes. This application makes use of engineered TAL effectors that are fused to the cleavage domain of FokI endonucleases for the targeted insertion of desirable genes into plants.

Tissue: any part of a plant that is used to produce a food. A tissue can be a tuber of a potato, a root of a sweet potato, or a seed of a maize plant.

Transcriptional terminators: The expression DNA constructs of the present invention typically have a transcriptional termination region at the opposite end from the transcription initiation regulatory region. The transcriptional termination region may be selected, for stability of the mRNA to enhance expression and/or for the addition of polyadenylation tails added to the gene transcription product. Translation of a nascent polypeptide undergoes termination when any of the three chain-termination codons enters the A site on the ribosome. Translation termination codons are UAA, UAG, and UGA. In the instant invention, transcription terminators are derived from either a gene or, more preferably, from a sequence that does not represent a gene but intergenic DNA. For example, the terminator sequence from the potato ubiquitin gene may be used.

Transfer DNA (T-DNA): a transfer DNA is a DNA segment delineated by either T-DNA borders or P-DNA borders to create a T-DNA or P-DNA, respectively. A T-DNA is a genetic element that is well-known as an element capable of integrating a nucleotide sequence contained within its borders into another genome. In this respect, a T-DNA is flanked, typically, by two “border” sequences. A desired polynucleotide of the present invention and a selectable marker may be positioned between the left border-like sequence and the right border-like sequence of a T-DNA. The desired polynucleotide and selectable marker contained within the T-DNA may be operably linked to a variety of different, plant-specific (i.e., native), or foreign nucleic acids, like promoter and terminator regulatory elements that facilitate its expression, i.e., transcription and/or translation of the DNA sequence encoded by the desired polynucleotide or selectable marker.

Transformation of plant cells: A process by which a nucleic acid is stably inserted into the genome of a plant cell. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols such as ‘refined transformation’ or ‘precise breeding’, viral infection, whiskers, electroporation, microinjection, polyethylene glycol-treatment, heat shock, lipofection and particle bombardment.

Transgenic plant: a transgenic plant of the present invention is one that comprises at least one cell genome in which an exogenous nucleic acid has been stably integrated. According to the present invention, a transgenic plant is a plant that comprises only one genetically modified cell and cell genome, or is a plant that comprises some genetically modified cells, or is a plant in which all of the cells are genetically modified. A transgenic plant of the present invention may be one that comprises expression of the desired polynucleotide, i.e., the exogenous nucleic acid, in only certain parts of the plant. Thus, a transgenic plant may contain only genetically modified cells in certain parts of its structure.

Variant: a “variant,” as used herein, is understood to mean a nucleotide or amino acid sequence that deviates from the standard, or given, nucleotide or amino acid sequence of a particular gene or protein. The terms, “isoform,” “isotype,” and “analog” also refer to “variant” forms of a nucleotide or an amino acid sequence. An amino acid sequence that is altered by the addition, removal or substitution of one or more amino acids, or a change in nucleotide sequence, may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. “Variant” may also refer to a “shuffled gene” such as those described in Maxygen-assigned patents.

It is understood that the present invention is not limited to the particular methodology, protocols, vectors, and reagents, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise. Thus, for example, a reference to “a gene” is a reference to one or more genes and includes equivalents thereof known to those skilled in the art and so forth. Indeed, one skilled in the art can use the methods described herein to express any native gene (known presently or subsequently) in plant host systems.

The following examples are set forth as representative of specific and preferred embodiments of the present invention. These examples are not to be construed as limiting the scope of the invention in any manner. It should be understood that many variations and modifications can be made while remaining within the spirit and scope of the invention.

### EXAMPLES

### Example 1

### Method for Targeted Insertion

A preferred target site for gene insertion is within an intron positioned in the untranslated 5′-leader region of the potato's ubiquitin-7 (Ubi7) gene. Potato is tetraploid and contains four copies of this gene; the copies are identical or near-identical. The Ubi7 genes are expressed at high levels in a near-constitutive manner, which suggests that they are located in regions that promote transcriptional activity. Sequences positioned within a transfer DNA are therefore expected to be effectively expressed. Furthermore, insertional inactivation of one of the Ubi7 genes is not expected to cause any quality or agronomic issues because potato still contains three functionally-active copies of the gene.

DNA segments were inserted into the intron sequence of the ubiquitin-7 gene according to the following steps:

(1) TAL effectors were designed to bind to sequences within the intron, which is (a) more than about 25-bp upstream from the region comprising branch site (consensus=CU(A/G)A(C/U)), pyrimidine-rich (=AT-rich) sequence, and intron/exon junction (consensus=CAGG), and (b) more than about 50-bp downstream from the splice donor site at the exon/intron junction (consensus=AGGT).

(2) A binary vector was created for transient expression of the TAL effectors in plant cells. This vector contains (a) a single right border but no left border; (b) two TAL effector genes operably linked to strong constitutive promoters; and (c) an expression cassette for the isopentenyl transferase (ipt) gene involved in cytokinin production. Stable transformation can be selected against because it would result in integration of the entire vector and, consequently, produce stunted shoots that overexpress cytokinins and are unable to produce roots.

(3) A second binary vector was created for stable transformation with a transfer DNA comprising genetic elements from potato delineated by borders: (a) right border; (b) part of the intron of the Ubi7 promoter, starting from the sequence between targeted TAL binding sites; (c) Ubi7 monomer-encoding sequence; (d) modified acetolactate synthase (ALS) gene that is insensitive to at least one ALS inhibitor selected from the group including sulfonylureas, imidazolinones, triazolopyrimidines, pyrimidinyl oxybenzoates, and sulfonylamino carbonyl triazolinones; (e) terminator of the ubiquitin-3 gene; (f) silencing cassette targeting the asparagine synthase 1 (Asn1), polyphenol oxidase (Ppo), and vacuolar invertase (Inv) genes; (g) late blight resistance gene Vnt1, operably linked to its native promoter and terminator sequences; and (h) left border. The vector backbone contains, apart from sequences required for maintenance and selection in E. coli and A. tumefaciens, an expression cassette for the ipt gene.

(4) The two binary vectors were separately introduced into the A. tumefaciens AGL-1 strain.

(5) Potato stem explants were co-infected with the two strains from step (4) and then co-cultivated for two days.

(6) Explants were transferred to media containing selection agents that kill Agrobacterium.

(7) Two weeks after transformation, the explants were again transferred to media also containing an ALS inhibitor.

(8) Herbicide resistant shoots arising from the explants within the next three months were transferred to root-inducing media and analyzed by PCR for the presence of a junction between the Ubi7 promoter and the modified ALS gene. At least 80% of regenerated plants contained such a junction.

(9) PCR-positive plants were regenerated, propagated, and evaluated for late blight resistance, reduced asparagine levels in tubers, black spot bruise tolerance, and reduced cold-induced sweetening.

The next examples describe aspects of the method.

### Example 2

### Imazamox Kill-Curve Essay

To determine the concentration of imazamox needed to kill untransformed potato cells, Ranger Russet internode stem explants were transformed with the binary vector pSIM1331. This vector contains (a) an expression cassette for the selectable marker gene nptII inserted between borders and (b) an expression cassette for the ipt gene in the backbone. The strain used to mediate transformation was Agrobacterium strain LBA4404, grown to an OD600 of 0.2. Following a 10 minute inoculation period, the explants were transferred to co-culture medium and placed in a Percival growth chamber at 24° C. under filtered light for 48 hours. Inter-node explants were transferred to hormone-free medium (HFM) containing the antibiotic timentin but lacking imazamox. Petri plates were place in Percival growth chamber at 24° C. and a 16 h photoperiod.

After two weeks, the inter-node explants were transferred to HFM containing timentin and five treatment concentrations (0, 0.5, 1.0, 1.5 & 2.0 mg/l) of the plant selection herbicide imazamox. Each treatment consisted of 3 replicates with each replicate containing ˜20 inter-node explants per Petri plate. Petri plates were placed in Percival growth chamber at 24° C. and a 16 h photoperiod. Inter-node explants were subcultured every 2 weeks to fresh HFM containing the respective treatment concentration of imazamox to encourage any regeneration of shoots and reduce any Agrobacterium over-growth.

Results indicated that a small number of inter-node explants in all imazamox treatment concentrations exhibited some Ipt meristamatic callus growth and primary shoot formation. However, no fully developed normal shoots arose in any of the imazamox treatment concentrations. Based upon these results, it was determined that 2.0 mg/l imazamox is the optimal concentration for in vitro selection. Optimal concentration is defined as the concentration of a selective agent that allows cell growth to some degree but does not allow regeneration of fully developed shoots.

The co-culture medium included 0.444 g/l Murashige & Skoog modified basal medium with Gamborg vitamins (M404; PhytoTechnology Laboratories), 30 g/l sucrose (S24060; Research Products International Corp.) and 6.0 g/l agar (S20400; Research Products International Corp.), and had pH 5.7

The hormone-free medium (HFM) included 4.44 g/l Murashige & Skoog modified basal medium with Gamborg vitamins (M404; PhytoTechnology Laboratories), 30 g/l sucrose (S24060; Research Products International Corp.), 300 mg/l timentin and 2.0 g/l Gelzan (G024; Caisson), and had pH 5.7

### Example 3

### Transformation and Regeneration of Potato Plants from Stem Explants Single Strain Approach

(1) 3-4-week old in vitro Ranger Russet potato plants growing on stock medium comprising 2.22 g/l Murashige & Skoog modified basal medium with Gamborg vitamins (M404; PhytoTechnology Laboratories), 15 g/l sucrose (S24060; Research Products International Corp.) and 2.0 g/l Gelzan (G024; Caisson) at pH 5.7, were used.

(2) The leaves and node sections were removed and inter-node stem portions were isolated. The inter-node stem portions were cut into 3-5 mm explants sections and placed in 15 ml of MS liquid medium containing 4.44 g/l Murashige & Skoog modified basal medium with Gamborg vitamins (M404; PhytoTechnology Laboratories), and 30 g/l sucrose (S24060; Research Products International Corp.) at pH 5.7.

(3) Agrobacterium (LBA4404) derived from a single colony containing a binary vector TAL effector cassette and a binary vector gene-of-interest cassette was grown overnight in Luria Broth at 28° C. in a shaking incubator. The next day the bacterial solution was pelleted and resuspended to 0.2 OD600 in MS liquid medium.

(4) Stem explants were incubated in the bacterial solution for 10 minutes at room temperature and blotted dry on sterile filter paper to remove excess of bacteria.

(5) The inoculated stem explants were placed on co-culture medium without selection in a Percival growth chamber for 48 h under filtered light. The co-culture medium contained 0.444 g/l Murashige & Skoog modified basal medium with Gamborg vitamins (M404; PhytoTechnology Laboratories), 30 g/l sucrose (S24060; Research Products International Corp.) and 6.0 g/l agar (S20400; Research Products International Corp.) at pH 5.7

(6) The stem explants were transferred to either callus induction hormone medium (CIHM) or hormone-free medium (HFM) containing antibiotics (timentin) and without plant selection. Petri plates were placed in a Percival growth chamber at 24° C. with a 16 h photoperiod. The CIHM contained 4.44 g/l Murashige & Skoog modified basal medium with Gamborg vitamins (M404; PhytoTechnology Laboratories), 30 g/l sucrose (S24060; Research Products International Corp.), 2.5 mg/l zeatin riboside, 0.1 mg/l NAA, 300 mg/l timentin and 6.0 g/l agar (S20400; Research Products International Corp.) at pH 5.7. The HFM contained 4.44 g/l Murashige & Skoog modified basal medium with Gamborg vitamins (M404; PhytoTechnology Laboratories), 30 g/l sucrose (S24060; Research Products International Corp.), 300 mg/l timentin and 2.0 g/l Gelzan (G024; Caisson) at pH 5.7

(7) After two weeks, the stem explants were transferred to either callus induction hormone medium (CIHM) or hormone-free medium (HFM) containing antibiotics (timentin) and plant selection. Petri plates were placed in a Percival growth chamber at 24° C. with a 16 h photoperiod. The CIHM contained 4.44 g/l Murashige & Skoog modified basal medium with Gamborg vitamins (M404; PhytoTechnology Laboratories), 30 g/l sucrose (S24060; Research Products International Corp.), 2.5 mg/l zeatin riboside, 0.1 mg/l NAA, 300 mg/l timentin, 2.0 mg/l imazamox and 6.0 g/l agar (S20400; Research Products International Corp.) at pH 5.7. The HFM contained 4.44 g/l Murashige & Skoog modified basal medium with Gamborg vitamins (M404; PhytoTechnology Laboratories), 30 g/l sucrose (S24060; Research Products International Corp.), 300 mg/l timentin, 2.0 mg/l imazamox and 2.0 g/l Gelzan (G024; Caisson) at pH 5.7.

(8) Four weeks post-transformation, the stem explants were transferred to either Shoot induction hormone medium (SIHM) or hormone-free medium (HFM) containing antibiotics (timentin) and plant selection. Petri plates were placed in a Percival growth chamber at 24° C. with a 16 h photoperiod. Stem explants were sub-cultured every 2-4 weeks to fresh SIHM or HFM to encourage full regeneration of shoots. The SIHM contained 4.44 g/l Murashige & Skoog modified basal medium with Gamborg vitamins (M404; PhytoTechnology Laboratories), 30 g/l sucrose (S24060; Research Products International Corp.), 2.5 mg/l zeatin riboside, 0.3 mg/l GA3, 300 mg/l timentin, 2.0 mg/l imazamox and 6.0 g/l agar (S20400; Research Products International Corp.) at pH 5.7. The HFM contained 4.44 g/l Murashige & Skoog modified basal medium with Gamborg vitamins (M404; PhytoTechnology Laboratories), 30 g/l sucrose (S24060; Research Products International Corp.), 300 mg/l timentin, 2.0 mg/l imazamox and 2.0 g/l Gelzan (G024; Caisson) at pH 5.7.

(9) Fully developed shoots were propagated for future testing and analysis.

### Example 4

### Transformation and Regeneration of Potato Plants from Stem Explants Double Strain Approach

(1) 3-4 week-old in vitro Ranger Russet potato plants growing on stock medium containing 2.22 g/l Murashige & Skoog modified basal medium with Gamborg vitamins (M404; PhytoTechnology Laboratories), 15 g/l sucrose (S24060; Research Products International Corp.) and 2.0 g/l Gelzan (G024; Caisson) at pH 5.7 were used.

(2) The leaves and node sections were removed and inter-node stem portions were isolated. The inter-node stem portions were cut into 3-5 mm explants sections and placed in 15 ml of MS liquid medium containing 4.44 g/l Murashige & Skoog modified basal medium with Gamborg vitamins (M404; PhytoTechnology Laboratories), and 30 g/l sucrose (S24060; Research Products International Corp.) at pH 5.7.

(3) Two separate Agrobacterium strains (LBA4404), each derived from a single colony, one containing a binary vector comprising a TAL effector cassette and the other containing a binary vector comprising a gene-of-interest cassette, were grown overnight in Luria Broth at 28° C. in a shaking incubator. The next day, each separate bacterial solution was pelleted and re-suspended to 0.2 OD600 in MS liquid medium.

(4) Stem explants were incubated in a single combined bacterial solution that consisted of equal volumes from each individual bacterial solution (co-transformation) for 10 minutes at room temperature and blotted dry on sterile filter paper to remove excess of bacteria.

(5) The inoculated stem explants were placed on co-culture medium without selection in a Percival growth chamber for 48 h under filtered light. The co-culture medium contained 0.444 g/l Murashige & Skoog modified basal medium with Gamborg vitamins (M404; PhytoTechnology Laboratories), 30 g/l sucrose (S24060; Research Products International Corp.) and 6.0 g/l agar (S20400; Research Products International Corp.) at pH 5.7.

(6) The stem explants were transferred to either callus induction hormone medium (CIHM) or hormone-free medium (HFM) containing antibiotics (Timentin) and without plant selection. The Petri plates were placed in a Percival growth chamber at 24° C. with a 16 h photoperiod. The CIHM contained 4.44 g/l Murashige & Skoog modified basal medium with Gamborg vitamins (M404; PhytoTechnology Laboratories), 30 g/l sucrose (S24060; Research Products International Corp.), 2.5 mg/l Zeatin Riboside, 0.1 mg/l NAA, 300 mg/l Timentin and 6.0 g/l agar (S20400; Research Products International Corp.) at pH 5.7. The HFM contained 4.44 g/l Murashige & Skoog modified basal medium with Gamborg vitamins (M404; PhytoTechnology Laboratories), 30 g/l sucrose (S24060; Research Products International Corp.), 300 mg/l Timentin and 2.0 g/l Gelzan (G024; Caisson) at pH 5.7.

(7) After two weeks, the stem explants were transferred to either callus induction hormone medium (CIHM) or hormone-free medium (HFM) containing antibiotics (Timentin) and plant selection. The Petri plates were placed in a Percival growth chamber at 24° C. with a 16 h photoperiod. The CIHM contained 4.44 g/l Murashige & Skoog modified basal medium with Gamborg vitamins (M404; PhytoTechnology Laboratories), 30 g/l sucrose (S24060; Research Products International Corp.), 2.5 mg/l Zeatin Riboside, 0.1 mg/l NAA, 300 mg/l Timentin, 2.0 mg/l imazamox and 6.0 g/l agar (S20400; Research Products International Corp.) at pH 5.7. The HFM contained 4.44 g/l Murashige & Skoog modified basal medium with Gamborg vitamins (M404; PhytoTechnology Laboratories), 30 g/l sucrose (S24060; Research Products International Corp.), 300 mg/l Timentin, 2.0 mg/l imazamox and 2.0 g/l Gelzan (G024; Caisson) at pH 5.7.

(8) Four weeks post-transformation, the stem explants were transferred to either Shoot induction hormone medium (SIHM) or hormone-free medium (HFM) containing antibiotics (Timentin) and plant selection. The Petri plates were placed in a Percival growth chamber at 24° C. with a 16 h photoperiod. Stem explants were subcultured every 2-4 weeks to fresh SIHM or HFM to encourage full regeneration of shoots. The SIHM contained 4.44 g/l Murashige & Skoog modified basal medium with Gamborg vitamins (M404; PhytoTechnology Laboratories), 30 g/l sucrose (S24060; Research Products International Corp.), 2.5 mg/l Zeatin Riboside, 0.3 mg/l GA3, 300 mg/l Timentin, 2.0 mg/l imazamox and 6.0 g/l agar (S20400; Research Products International Corp.) at pH 5.7. The HFM contained 4.44 g/l Murashige & Skoog modified basal medium with Gamborg vitamins (M404; PhytoTechnology Laboratories), 30 g/l sucrose (S24060; Research Products International Corp.), 300 mg/l Timentin, 2.0 mg/l imazamox and 2.0 g/l Gelzan (G024; Caisson) at pH 5.7

9) Fully developed shoots were propagated for future testing and analysis.

### Example 5

### Target Site Sequence in Potato Ranger, Burbank and Atlantic Cultivars

To determine if the target region (5′ region of the Ubi7 promoter intron) is conserved across different potato cultivars, primer pair HD175F1 and HD175R1 (SEQ ID NO: 1 and SEQ ID NO: 2) were designed and used to amplify target region from the potato varieties Ranger, Burbank and Atlantic. The amplified fragments were cloned into pGEMT-easy vector and sequenced. Sequence results showed that the target region is identical for all varieties tested. The ubi7 promoter intron sequence is represented by SEQ ID NO: 3.

### Example 6

### Design of TAL Effectors

A pair of TAL effectors was designed to target the selected region. Forward and reverse TALE recognition sites are listed as SEQ ID NO: 4 and SEQ ID NO: 5, respectively. The TALE scaffold was Hax3, a member of the AvrBs3 family that was identified in Brassicaceae pathogen X. campestris pv. Armoraciae strain 5. The modification made on this scaffold included: (a) the C-terminal activation domain of original Hax3 was truncated; (b) a nuclear localization sequence from SV40 virus was added at the N terminal of truncated Hax3 protein; (c) a codon optimization was performed on original Hax3 DNA sequence; (d) original 11.5 repeat variable diresidues (RVD) were replaced by 16.5 RVDs corresponding to the targeting sites; (e) a catalytic domain of Fok1 nuclease was added at the C-terminal of modified Hax3 scaffold.

The organization of effector proteins is shown in FIG. 5. The DNA and protein sequences of final forward and reverse TALEs are listed as SEQ ID NOS: 6, 7, 8 and 9.

### Example 7

### Vector for DNA Transfer

The transfer DNA consisted of potato-derived genetic elements and was delineated by T-DNA-like borders. It included three cassettes from left border to right border: (a) a late blight resistant cassette; (b) a tuber-specific silencing cassette targeting three genes: the ASN1 gene involved in asparagine formation; the acidic invertase (INV) gene associated with hydrolysis of sucrose; and the polyphenol oxidase (PPO) gene that encodes the enzyme oxidizing polyphenols upon impact bruise; and (c) a promoter-less mutated potato acetolactate synthase (ALS) gene (with W563L AND S642I substitutions) that was hypothesized to confer resistance to ALS inhibiting herbicides when over-expressed.

The transfer DNA was designed to be inserted into the intron region positioned within the leader of one of potato's four Ubi7 genes, so that the associated Ubi7 promoter would drive expression of the ALS gene and confer resistance against ALS inhibitor-type herbicides.

Since the Ubi7 monomer plays an important role in protein stabilization, the coding sequence, preceded by part of the intron, was fused in frame to the ALS gene. Insertion of the transfer DNA into a binary vector created the plasmid pSIM2168. The organization of the transfer DNA is illustrated in FIG. 1. The DNA and protein sequences of wild type and mutated ALS gene are represented by SEQ ID NOS: 10, 11, 12 and 13. The whole transfer DNA sequence in pSIM2168 is represented by SEQ ID NO: 14.

### Example 8

### Vector for TAL Effectors

Each TAL effector, forward or reverse, was driven by a constitutive (35s or FMV) promoter and followed by a terminator (Nos or Ocs), to form two separate plant expression cassettes. The two cassettes were cloned into a binary vector to form the pSIM2170 as shown in FIG. 2. This binary vector had only one border and contained an ipt gene expression cassette so that it was possible to select against stable integration of the effector genes.

### Example 9

### The Right Border Upstream the Ubi7 Intron 5′ Region Supports DNA Transfer

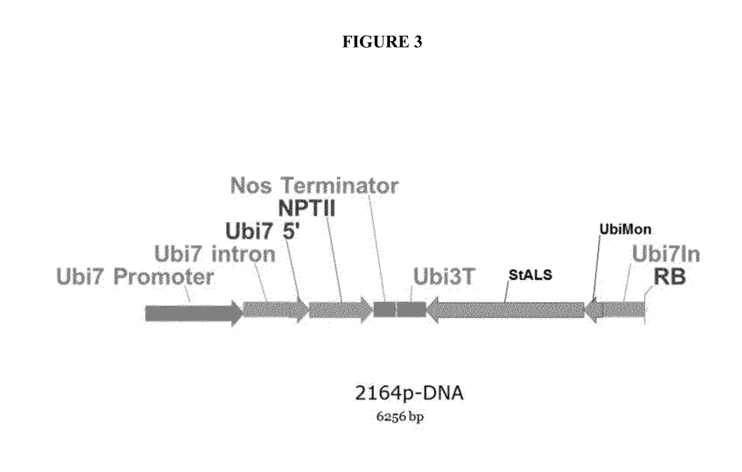

Because efficacy of the border as primary cleavage site is dependent, in part, on flanking DNA sequences, a right border upstream the Ubi7 intron 5′region was tested for its ability to support DNA transfer. For this purpose, a DNA fragment comprising the right border/intron sequence upstream from the Ubi7 monomer and modified ALS gene was cloned into the binary vector pSIM123-F to form pSIM2164. Vector pSIM123-F contained an expression cassette for the selectable marker gene nptII, but lacked the borders needed to transfer this cassette into plant cells (see FIG. 3). Nevertheless, infection of explants with an Agrobacterium strain carrying the pSIM2164 generated the same number of kanamycin resistant shoots per explant as a positive control (infection with a strain carrying the nptII gene positioned within T-DNA borders).

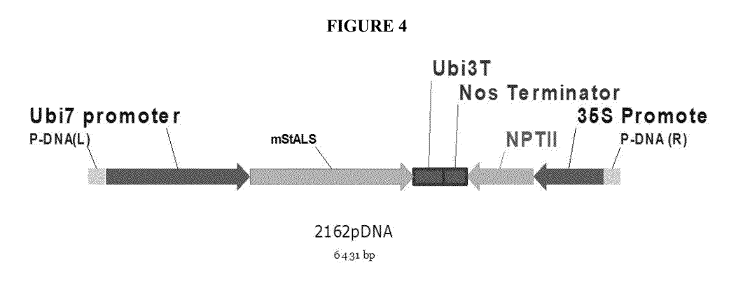

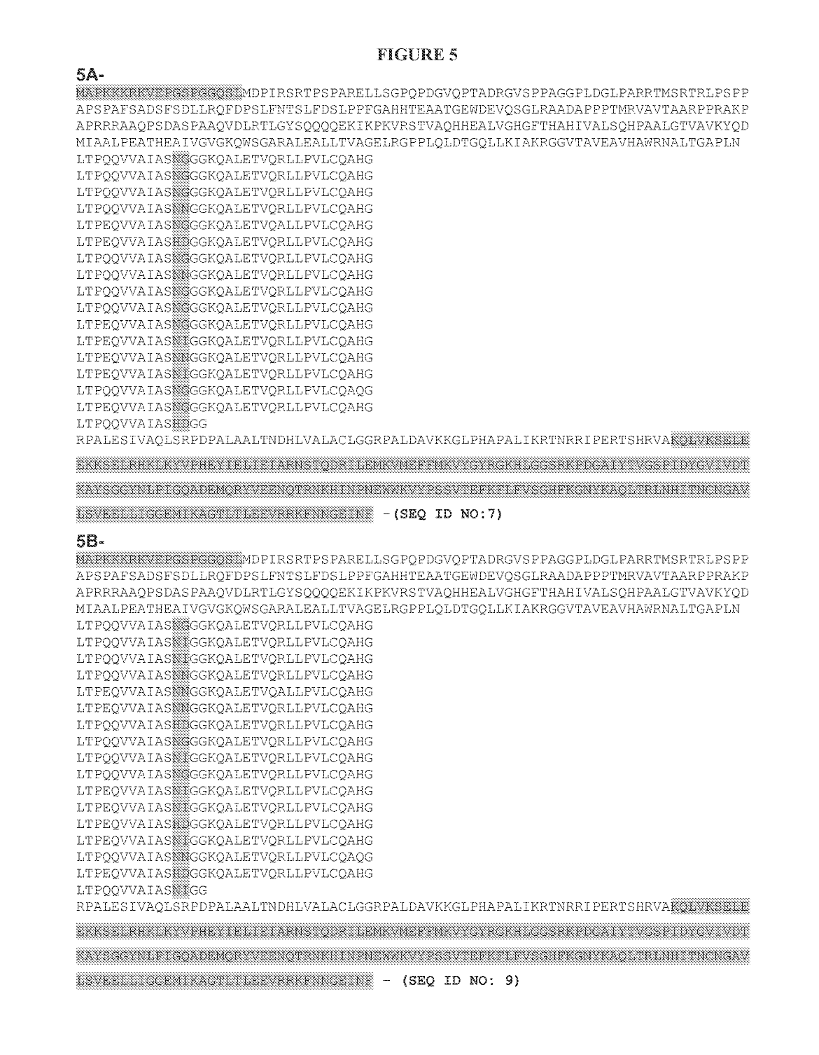

To test the efficiency of the mutated ALS gene in conferring imazamox resistance to potato, a vector carrying a Ubi7::ALS cassette (pSIM2162, see FIG. 4) was created.

Transformation with this vector yielded herbicide resistant plants that were confirmed by PCR to contain the Ubi7::ALS cassette.

### Example 10

### Vector design for transient transformation in N. benthamiana

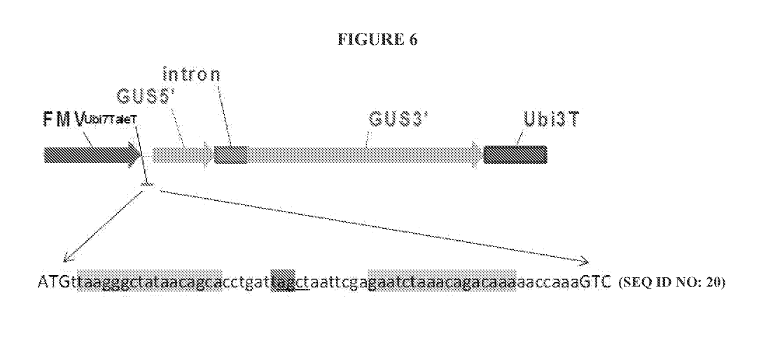

To test the efficiency of the specifically designed TALEs in vivo, a vector with the target sequence (part of the Ubi7 intron) was designed. This vector, pSIM2167, was co-transformed with the vector carrying the effectors into N. benthamiana. As shown in FIG. 6, the target sequence contained the forward and reverse recognition sites positioned immediately downstream from the start codon of the GUS reporter gene. A stop codon between the two recognition sequences and in frame with the GUS coding sequence rendered the GUS coding sequence inactive. If the TALEs bind their designed recognition sites and cleave in the intermediary sequence, subsequent repair would be expected to occasionally eliminate the stop codon without altering the reading frame, thus restoring GUS function. Such events could be visualized by histochemically staining the N. benthamiana leaves, about 4 days after infiltration.

The target sequence region can also be PCR amplified and sequenced to identify TALE mediated mutations. However, direct PCR and cloning of the target sequence would yield an un-modified target sequence because of the possible low efficiency of transformation. Therefore, the isolated DNA was first digested with the AluI enzyme, which cleaves the AGCT restriction site located between the two TALE recognition sites. After amplification, the PCR products were again digested with AluI to further enrich the mutated target sequence for downstream cloning and sequencing analyses. The entire sequence of FMV-target-GUS-Nos cassette is represented by SEQ ID NO: 15. The PCR primers used for amplifying the target sequence are represented by SEQ ID NO: 16 and SEQ ID NO: 17.

### Example 11

### Agrobacterium Transformation and N. benthamiana Infiltration

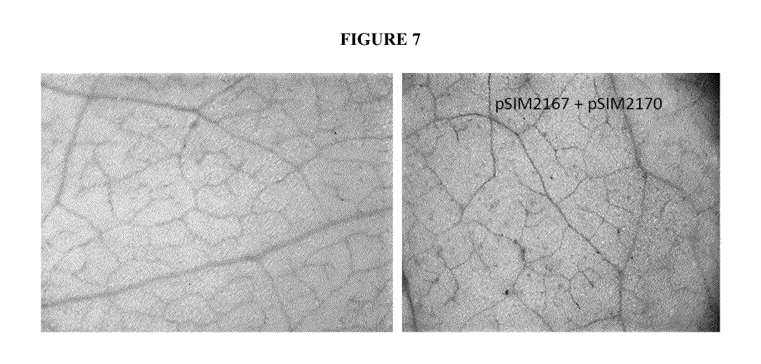

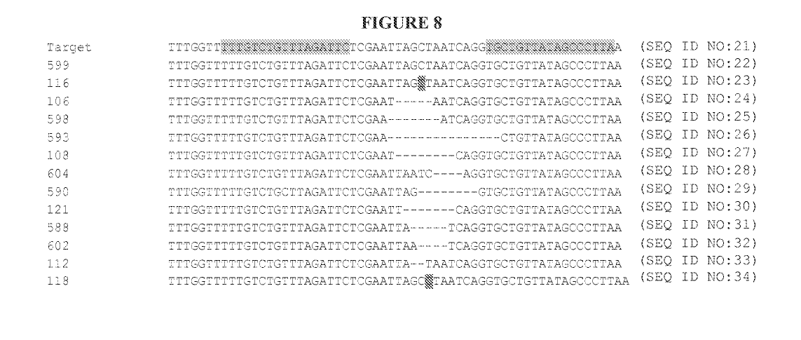

The designed vectors were transformed into Agrobacterium strain AGL1 and tested for vector stability. Four to six days after infiltration, leaf discs from infiltrated tissue were collected for GUS staining assay and DNA isolation. Isolated DNA was digested with the AluI enzyme and used as template for target region amplification and further cloning and sequencing. As shown in FIG. 7, GUS staining was observed in co-infiltrated tissue (right panel) but not in the tissue infiltrated by target vector alone (left panel). Further sequence analyses showed in FIG. 8 also confirmed that the target sequence was modified by TALEs.

### Example 12

### Genotyping of Stable Transformants

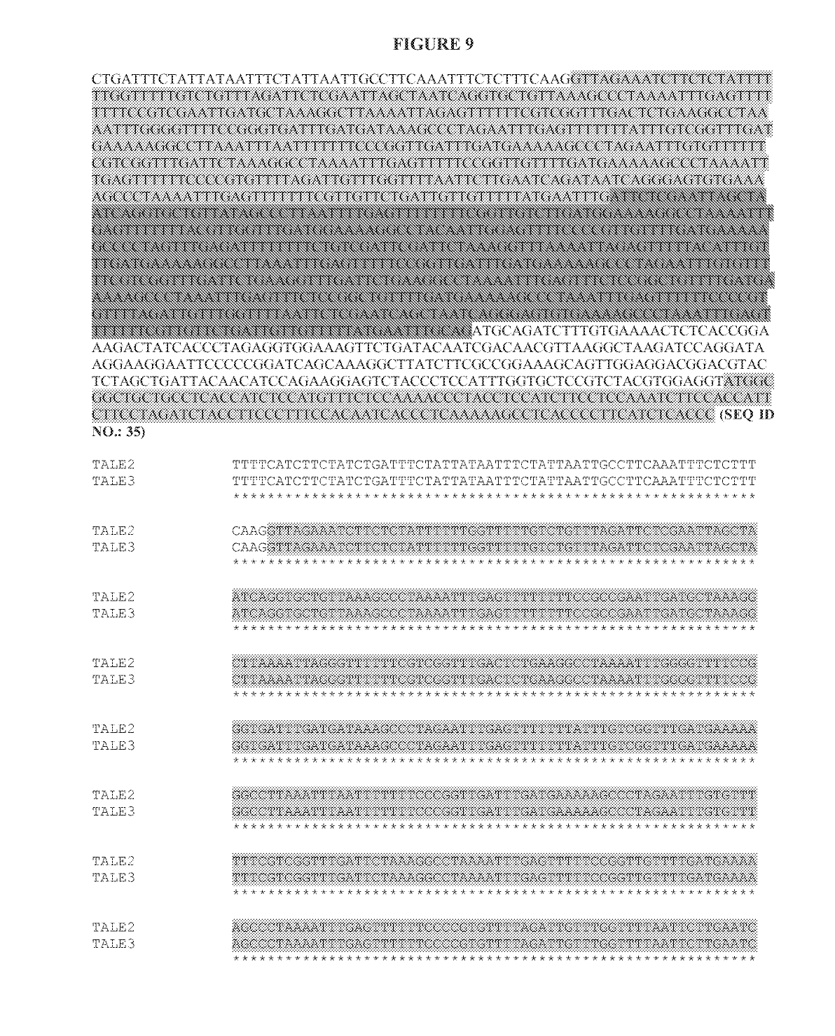

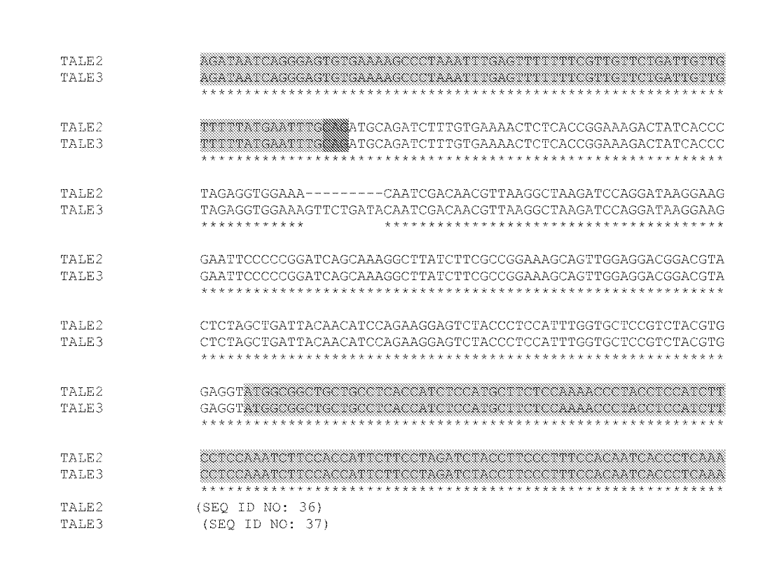

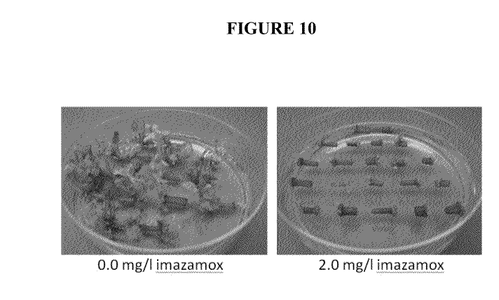

Primer pairs HD208 F1 and R1 were designed to genotype herbicide resistant transformants. The forward primer is located in the promoter region of Ubi7 gene, and the reverse primer is located in ALS coding region. The primer pair is targeted-insertion specific primer because only if the transfer DNA is inserted into the designed position, the primer pair will amplify a fragment. PCR analysis of the independent herbicide resistant lines from the co-transfromation of pSIM2170 and pSIM2168 did amplify fragments. These fragments were cloned and sequenced. As shown in FIG. 9, in one line, TALE1, the fragment contained part of the transfer DNA cassette, including the partial Ubi7 intron, the Ubi7 monomer and part of the ALS coding region, flanked by potato genome sequence. Sequence blast showed that the flanked potato genome is the promoter region of an Ubi7 like gene located on chromosome 7 which also contains very similar recognition sites of the designed TALE. In another two lines, TALE2 and TALE3, the transfer DNA cassettes were inserted into the same genomic loci as in TALE1, except that intron portions of the transfer DNA cassette were largely deleted. The TALE2 and TALE3 lines were very similar, except that in TALE2 there was a 9 by deletion in the Ubi7 monomer.

### Example 13

### Characterization of Stable Transformed Lines for Targeted Insertion

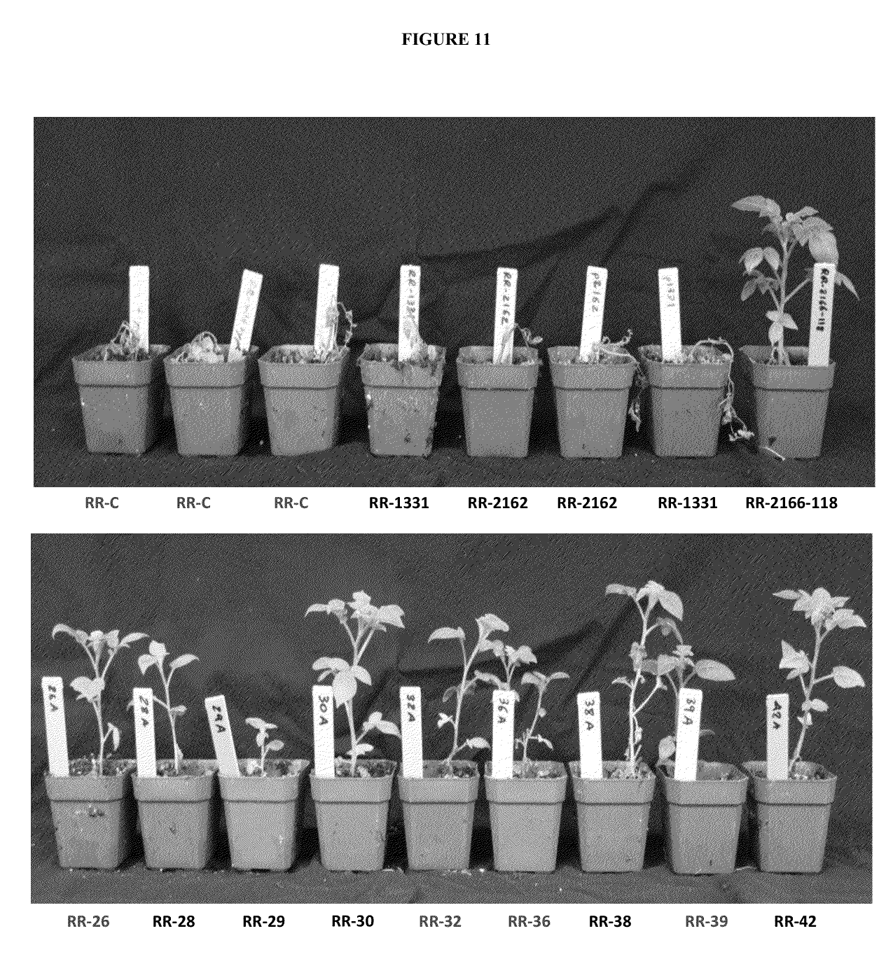

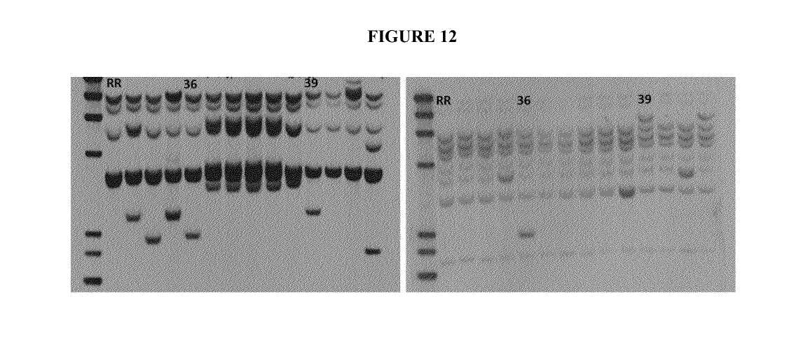

The data and results described above indicated that the targeted insertion of an intended DNA segment was successful. Herbicide resistant Ranger Russet (RR) lines from the co-transformation of pSIM2170 and pSIM2168 were propagated and transferred to soil for following tests/analyses. Specifically, the transformed lines were tested for resistance to Late blight diseases challenge, by determining the activity of the enzyme polyphenol oxidase, and running southern analyses for copy number of both silencing and Vnt1 cassettes. For diseases assay, plantlets in soil for three weeks were inoculated with P. infestans late blight strain US8 BF6 for the development of disease symptom. For Southern blot analyses, 3 μg DNA isolated from leaf tissues were digested by HindIII restriction enzyme, run on 0.7% agarose gel, transferred to positive charged nylon membrane and hybridized with Dig labeled probes either for invertase fragment in silencing cassette or for Vnt1 promoter in Vnt1 expression cassette. Four lines were identified and summarized in Table 1 below. These lines were late blight resistant (see FIG. 11) and had a single copy for both cassettes (see FIG. 12). Each extra band in lines RR-36 and RR-39, as compared to RR control lines, indicated the presence of a single copy of the transgene. (Data for line RR-26 and RR-32 are not shown).

TABLE 1
Line Characterization for Targeted Insertion.

| Line Number | Late Blight | Invertase Copy No. | Vnt1 Copy No. |
|---|---|---|---|
| Ranger control | susceptible | 0 | 0 |
| RR-26 | resistant | 1 | 1 |
| RR-32 | resistant | 1 | 1 |
| RR-36 | resistant | 1 | 1 |
| RR-38 | resistant | 1 | 1 |

SEQUENCE LISTING
SEQ ID NO: 1
TCCTAATTTTCCCCACCACA
SEQ ID NO: 2
AACAGCCGGAGAAACTCAAA
SEQ ID NO: 3 UBI7 PROMOTER INTRON
GTTAGAAATCTTCTCTATTTTTGGTTTTTGTCTGTTTAGATTCTCGAATTAGCTAATCAGGTGCTGTTATAGCCCTT
AATTTTGAGTTTTTTTTCGGTTGTCTTGATGGAAAAGGCCTAAAATTTGAGTTTTTTTACGTTGGTTTGATGGAAAA
GGCCTACAATTGGAGTTTTCCCCGTTGTTTTGATGAAAAAGCCCCTAGTTTGAGATTTTTTTTCTGTCGATTCGATT
CTAAAGGTTTAAAATTAGAGTTTTTACATTTGTTTGATGAAAAAGGCCTTAAATTTGAGTTTTTCCGGTTGATTTGA
TGAAAAAGCCCTAGAATTTGTGTTTTTTCGTCGGTTTGATTCTGAAGGCCTAAAATTTGAGTTTCTCCGGCTGTTTT
GATGAAAAAGCCCTAAATTTGAGTTTCTCCGGCTGTTTTGATGAAAAAGCCCTAAATTTGAGTTTTTTCCCCGTGTT
TTAGATTGTTTGGTTTTAATTCTCGAATCAGCTAATCAGGGAGTGTGAAAAGCCCTAAATTTGAGTTTTTTTCGTTG
TTCTGATTGTTGTTTTTATGAATTTGCAG
SEQ ID NO: 4 UBI7INTE3 (FORWARD) RECOGNITION SITE
(T) TTTGTCTGTTTAGATTC
SEQ ID NO: 5 UBI7INTE4 (REVERSE) RECOGNITION SITE
(T) TAAGGGCTATAACAGCA
SEQ ID NO: 6 E3 (FORWARD EFFECTOR) DNA
ATGGCTCCCAAAAAGAAGAGAAAGGTAGAACCAGGATCACCTGGTGGACAATCACTTATGGACCCAATACGAAGCAG
AACGCCATCACCAGCTAGGGAACTTCTCTCTGGACCACAGCCTGATGGAGTTCAGCCAACTGCAGATCGAGGTGTTT
CTCCGCCAGCCGGTGGCCCTTTAGATGGACTCCCAGCAAGAAGAACAATGTCCCGTACCAGACTCCCAAGTCCCCCT
GCCCCGTCGCCAGCCTTTTCAGCTGACTCCTTCTCTGATCTTCTTAGGCAATTTGACCCTTCTCTTTTCAATACATC
CCTTTTCGATTCACTTCCTCCTTTCGGCGCACATCATACTGAGGCAGCCACCGGCGAATGGGACGAAGTCCAAAGTG
GTTTAAGGGCAGCTGATGCTCCACCACCGACGATGAGAGTCGCTGTTACCGCCGCACGTCCTCCTAGAGCCAAGCCA
GCCCCTAGAAGACGAGCTGCGCAACCCTCCGATGCAAGCCCTGCAGCTCAAGTAGACCTTCGAACACTAGGTTACTC
CCAGCAACAACAAGAAAAAATAAAGCCAAAGGTTAGATCAACAGTTGCACAACATCACGAAGCCCTAGTCGGACACG
GATTTACACATGCTCATATCGTGGCTCTTTCACAACATCCTGCAGCTCTTGGAACAGTCGCTGTCAAATATCAGGAT
ATGATTGCTGCATTGCCAGAAGCTACTCACGAAGCTATCGTCGGAGTTGGGAAACAATGGTCAGGCGCAAGAGCATT
AGAGGCGCTTCTCACCGTAGCTGGTGAATTACGAGGTCCTCCACTCCAATTGGATACTGGGCAATTATTAAAAATCG
CTAAACGAGGTGGAGTCACTGCTGTCGAAGCCGTTCATGCATGGCGTAACGCTCTCACGGGGGCCCCACTAAACCTT
ACCCCACAACAAGTTGTGGCAATAGCTTCTAATGGTGGTGGTAAACAAGCCCTTGAGACGGTTCAAAGACTTCTACC
AGTTCTTTGTCAGGCACATGGATTGACCCCACAACAGGTCGTAGCAATCGCATCTAACGGAGGTGGTAAGCAAGCTC
TTGAAACGGTACAAAGATTACTTCCCGTGCTTTGTCAAGCTCATGGACTCACTCCTCAACAAGTGGTCGCTATTGCA
AGTAACGGTGGTGGAAAGCAAGCACTAGAAACCGTCCAACGACTCCTTCCTGTTCTCTGTCAAGCACATGGTTTGAC
TCCTCAGCAGGTCGTCGCAATTGCATCAAACAATGGAGGCAAACAAGCTTTAGAAACAGTACAAAGACTATTGCCCG
TTCTTTGCCAAGCGCATGGGTTAACTCCCGAACAAGTCGTTGCCATTGCAAGTAACGGAGGAGGTAAACAAGCTCTC
GAAACGGTTCAAGCACTTTTACCCGTTCTCTGTCAAGCACATGGACTCACACCTGAACAAGTAGTTGCTATCGCATC
GCATGATGGTGGAAAACAAGCACTGGAAACTGTACAAAGACTTTTGCCAGTTTTATGTCAAGCGCACGGTCTTACTC
CTCAACAAGTTGTCGCCATTGCCTCTAATGGAGGTGGAAAACAAGCTCTTGAAACTGTCCAGAGACTTCTGCCCGTT
CTATGTCAGGCTCATGGGCTAACCCCTCAACAGGTTGTTGCAATCGCATCTAATAATGGAGGAAAACAAGCTTTAGA
AACTGTCCAACGACTACTGCCCGTTCTCTGCCAAGCACACGGACTTACACCACAACAGGTTGTAGCTATAGCTAGCA
ATGGTGGCGGTAAACAGGCTTTGGAAACAGTACAGCGGCTTCTACCAGTCTTATGCCAAGCCCACGGGCTTACTCCT
CAACAAGTTGTCGCCATTGCCTCTAATGGAGGTGGAAAACAAGCTCTTGAAACTGTCCAGAGACTTCTGCCCGTTCT
ATGTCAGGCTCATGGGCTTACTCCTGAACAGGTTGTCGCAATAGCTTCAAACGGTGGCGGAAAACAAGCTCTTGAAA
CAGTGCAACGTCTCCTTCCCGTCCTCTGTCAGGCTCACGGACTTACGCCCGAACAAGTTGTTGCTATAGCTTCGAAT
ATTGGTGGAAAACAAGCTCTCGAAACCGTCCAAAGGCTCCTCCCAGTACTTTGCCAAGCACATGGATTAACCCCTGA
GCAAGTAGTTGCAATTGCCTCGAACAATGGAGGAAAGCAAGCATTAGAAACTGTTCAGAGACTTTTGCCTGTCCTGT
GTCAAGCCCACGGTCTTACACCAGAGCAGGTTGTCGCTATAGCTTCTAACATTGGTGGAAAGCAAGCTCTTGAGACT
GTGCAACGTTTGCTTCCAGTCCTCTGTCAAGCACACGGACTCACTCCACAACAGGTGGTTGCAATTGCTTCAAATGG
CGGTGGCAAACAAGCATTAGAGACTGTACAGAGACTACTTCCTGTTCTTTGTCAAGCACAAGGGCTCACCCCTGAGC
AGGTAGTCGCTATCGCCTCAAATGGTGGCGGGAAGCAGGCCCTGGAGACTGTTCAGAGACTACTGCCCGTCCTATGT
CAGGCTCACGGTCTAACACCACAACAAGTCGTCGCAATCGCTAGTCATGACGGAGGTCGACCTGCTCTAGAGTCGAT
AGTCGCACAACTATCACGACCTGATCCCGCTCTTGCAGCATTGACAAACGATCATTTAGTCGCACTTGCATGTTTAG
GAGGACGACCAGCACTTGATGCCGTTAAGAAAGGACTACCGCACGCCCCTGCATTGATTAAAAGAACAAACAGACGA
ATCCCGGAGAGAACTTCACATCGTGTAGCCAAGCAACTTGTCAAAAGTGAACTGGAGGAGAAGAAATCTGAACTTCG
TCATAAATTGAAATATGTGCCTCATGAATATATTGAATTAATTGAAATTGCCAGAAATTCCACTCAGGATAGAATTC
TTGAAATGAAGGTAATGGAATTTTTTATGAAAGTTTATGGATATAGAGGTAAACATTTGGGTGGATCAAGGAAACCG
GACGGAGCAATTTATACTGTCGGATCTCCTATTGATTACGGTGTGATCGTGGATACTAAAGCTTATAGCGGAGGTTA
TAATCTGCCAATTGGCCAAGCAGATGAAATGCAACGATATGTCGAAGAAAATCAAACACGAAACAAACATATCAACC
CTAATGAATGGTGGAAAGTCTATCCATCTTCTGTAACGGAATTTAAGTTTTTATTTGTGAGTGGTCACTTTAAAGGA
AACTACAAAGCTCAGCTTACACGATTAAATCATATCACTAATTGTAATGGAGCTGTTCTTAGTGTAGAAGAGCTTTT
AATTGGTGGAGAAATGATTAAAGCCGGCACATTAACCTTAGAGGAAGTGAGACGGAAATTTAATAACGGCGAGATAA
ACTTTTGA
SEQ ID NO: 7.
MAPKKKRKVEPGSPGGQSLMDPIRSRTPSPARELLSGPQPDGVQPTADRGVSPPAGGPLDGLPARRTMSRTRLPSPP
APSPAFSADSFSDLLRQFDPSLFNTSLFDSLPPFGAHHTEAATGEWDEVQSGLRAADAPPPTMRVAVTAARPPRAKP
APRRRAAQPSDASPAAQVDLRTLGYSQQQQEKIKPKVRSTVAQHHEALVGHGFTHAHIVALSQHPAALGTVAVKYQD
MIAALPEATHEAIVGVGKQWSGARALEALLTVAGELRGPPLQLDTGQLLKIAKRGGVTAVEAVHAWRNALTGAPLNL
TPQQVVAIASNGGGKQALETVQRLLPVLCQAHGLTPQQVVAIASNGGGKQALETVQRLLPVLCQAHGLTPQQVVAIA
SNGGGKQALETVQRLLPVLCQAHGLTPQQVVAIASNNGGKQALETVQRLLPVLCQAHGLTPEQVVAIASNGGGKQAL
ETVQALLPVLCQAHGLTPEQVVAIASHDGGKQALETVQRLLPVLCQAHGLTPQQVVAIASNGGGKQALETVQRLLPV
LCQAHGLTPQQVVAIASNNGGKQALETVQRLLPVLCQAHGLTPQQVVAIASNGGGKQALETVQRLLPVLCQAHGLTP
QQVVAIASNGGGKQALETVQRLLPVLCQAHGLTPEQVVAIASNGGGKQALETVQRLLPVLCQAHGLTPEQVVAIASN
IGGKQALETVQRLLPVLCQAHGLTPEQVVAIASNNGGKQALETVQRLLPVLCQAHGLTPEQVVAIASNIGGKQALET
VQRLLPVLCQAHGLTPQQVVAIASNGGGKQALETVQRLLPVLCQAQGLTPEQVVAIASNGGGKQALETVQRLLPVLC
QAHGLTPQQVVAIASHDGGRPALESIVAQLSRPDPALAALTNDHLVALACLGGRPALDAVKKGLPHAPALIKRTNRR
IPERTSHRVAKQLVKSELEEKKSELRHKLKYVPHEYIELIEIARNSTQDRILEMKVMEFFMKVYGYRGKHLGGSRKP
DGAIYTVGSPIDYGVIVDTKAYSGGYNLPIGQADEMQRYVEENQTRNKHINPNEWWKVYPSSVTEFKFLFVSGHFKG
NYKAQLTRLNHITNCNGAVLSVEELLIGGEMIKAGTLTLEEVRRKFNNGEINF
SEQ ID NO: 8. E4(REVERSE EFFECTOR) DNA
ATGGCTCCCAAAAAGAAGAGAAAGGTAGAACCAGGATCACCTGGTGGACAATCACTTATGGACCCAATACGAAGCAG
AACGCCATCACCAGCTAGGGAACTTCTCTCTGGACCACAGCCTGATGGAGTTCAGCCAACTGCAGATCGAGGTGTTT
CTCCGCCAGCCGGTGGCCCTTTAGATGGACTCCCAGCAAGAAGAACAATGTCCCGTACCAGACTCCCAAGTCCCCCT
GCCCCGTCGCCAGCCTTTTCAGCTGACTCCTTCTCTGATCTTCTTAGGCAATTTGACCCTTCTCTTTTCAATACATC
CCTTTTCGATTCACTTCCTCCTTTCGGCGCACATCATACTGAGGCAGCCACCGGCGAATGGGACGAAGTCCAAAGTG
GTTTAAGGGCAGCTGATGCTCCACCACCGACGATGAGAGTCGCTGTTACCGCCGCACGTCCTCCTAGAGCCAAGCCA
GCCCCTAGAAGACGAGCTGCGCAACCCTCCGATGCAAGCCCTGCAGCTCAAGTAGACCTTCGAACACTAGGTTACTC
CCAGCAACAACAAGAAAAAATAAAGCCAAAGGTTAGATCAACAGTTGCACAACATCACGAAGCCCTAGTCGGACACG
GATTTACACATGCTCATATCGTGGCTCTTTCACAACATCCTGCAGCTCTTGGAACAGTCGCTGTCAAATATCAGGAT
ATGATTGCTGCATTGCCAGAAGCTACTCACGAAGCTATCGTCGGAGTTGGGAAACAATGGTCAGGCGCAAGAGCATT
AGAGGCGCTTCTCACCGTAGCTGGTGAATTACGAGGTCCTCCACTCCAATTGGATACTGGGCAATTATTAAAAATCG
CTAAACGAGGTGGAGTCACTGCTGTCGAAGCCGTTCATGCATGGCGTAACGCTCTCACGGGGGCCCCACTAAACCTT
ACCCCACAACAAGTTGTGGCAATAGCTTCTAATGGAGGTGGTAAACAAGCCCTTGAGACGGTTCAAAGACTTCTACC
AGTTCTTTGTCAGGCACATGGATTGACCCCACAACAGGTCGTAGCAATCGCATCTAACATTGGTGGTAAGCAAGCTC
TTGAAACGGTACAAAGATTACTTCCCGTGCTTTGTCAAGCTCATGGACTCACTCCTCAACAAGTGGTCGCTATTGCA
AGTAATATTGGTGGAAAGCAAGCACTAGAAACCGTCCAACGACTCCTTCCTGTTCTCTGTCAAGCACATGGTTTGAC
TCCTCAGCAGGTCGTCGCAATTGCATCAAATAACGGAGGCAAACAAGCTTTAGAAACAGTACAAAGACTATTGCCCG
TTCTTTGCCAAGCGCATGGGTTAACTCCCGAACAAGTCGTTGCCATTGCAAGTAACAATGGAGGTAAACAAGCTCTC
GAAACGGTTCAAGCACTTTTACCCGTTCTCTGTCAAGCACATGGACTCACACCTGAACAAGTAGTTGCTATCGCATC
GAATAATGGTGGAAAACAAGCACTGGAAACTGTACAAAGACTTTTGCCAGTTTTATGTCAAGCGCACGGTCTTACTC
CTCAACAAGTTGTCGCCATTGCCTCTCATGATGGTGGAAAACAAGCTCTTGAAACTGTCCAGAGACTTCTGCCCGTT
CTATGTCAGGCTCATGGGCTAACCCCTCAACAGGTTGTTGCAATCGCATCTAATGGTGGAGGAAAACAAGCTTTAGA
AACTGTCCAACGACTACTGCCCGTTCTCTGCCAAGCACACGGACTTACACCACAACAGGTTGTAGCTATAGCTAGCA
ATATTGGCGGTAAACAGGCTTTGGAAACAGTACAGCGGCTTCTACCAGTCTTATGCCAAGCCCACGGGCTTACTCCT
CAACAAGTTGTCGCCATTGCCTCTAACGGAGGTGGAAAACAAGCTCTTGAAACTGTCCAGAGACTTCTGCCCGTTCT
ATGTCAGGCTCATGGGCTTACTCCTGAACAGGTTGTCGCAATAGCTTCAAACATTGGCGGAAAACAAGCTCTTGAAA
CAGTGCAACGTCTCCTTCCCGTCCTCTGTCAGGCTCACGGACTTACGCCCGAACAAGTTGTTGCTATAGCTTCGAAT
ATTGGTGGAAAACAAGCTCTCGAAACCGTCCAAAGGCTCCTCCCAGTACTTTGCCAAGCACATGGATTAACCCCTGA
GCAAGTAGTTGCAATTGCCTCGCACGATGGAGGAAAGCAAGCATTAGAAACTGTTCAGAGACTTTTGCCTGTCCTGT
GTCAAGCCCACGGTCTTACACCAGAGCAGGTTGTCGCTATAGCTTCTAATATCGGTGGAAAGCAAGCTCTTGAGACT
GTGCAACGTTTGCTTCCAGTCCTCTGTCAAGCACACGGACTCACTCCACAACAGGTGGTTGCAATTGCTTCAAATAA
TGGTGGCAAACAAGCATTAGAGACTGTACAGAGACTACTTCCTGTTCTTTGTCAAGCACAAGGGCTCACCCCTGAGC
AGGTAGTCGCTATCGCCTCACACGACGGCGGGAAGCAGGCCCTGGAGACTGTTCAGAGACTACTGCCCGTCCTATGT
CAGGCTCACGGTCTAACACCACAACAAGTCGTCGCAATCGCTAGTAATATTGGAGGTCGACCTGCTCTAGAGTCGAT
AGTCGCACAACTATCACGACCTGATCCCGCTCTTGCAGCATTGACAAACGATCATTTAGTCGCACTTGCATGTTTAG
GAGGACGACCAGCACTTGATGCCGTTAAGAAAGGACTACCGCACGCCCCTGCATTGATTAAAAGAACAAACAGACGA
ATCCCGGAGAGAACTTCACATCGTGTAGCCAAGCAACTTGTCAAAAGTGAACTGGAGGAGAAGAAATCTGAACTTCG
TCATAAATTGAAATATGTGCCTCATGAATATATTGAATTAATTGAAATTGCCAGAAATTCCACTCAGGATAGAATTC
TTGAAATGAAGGTAATGGAATTTTTTATGAAAGTTTATGGATATAGAGGTAAACATTTGGGTGGATCAAGGAAACCG
GACGGAGCAATTTATACTGTCGGATCTCCTATTGATTACGGTGTGATCGTGGATACTAAAGCTTATAGCGGAGGTTA
TAATCTGCCAATTGGCCAAGCAGATGAAATGCAACGATATGTCGAAGAAAATCAAACACGAAACAAACATATCAACC
CTAATGAATGGTGGAAAGTCTATCCATCTTCTGTAACGGAATTTAAGTTTTTATTTGTGAGTGGTCACTTTAAAGGA
AACTACAAAGCTCAGCTTACACGATTAAATCATATCACTAATTGTAATGGAGCTGTTCTTAGTGTAGAAGAGCTTTT
AATTGGTGGAGAAATGATTAAAGCCGGCACATTAACCTTAGAGGAAGTGAGACGGAAATTTAATAACGGCGAGATAA
ACTTTTGA
SEQ ID NO: 9. E4 PROTEIN
MAPKKKRKVEPGSPGGQSLMDPIRSRTPSPARELLSGPQPDGVQPTADRGVSPPAGGPLDGLPARRTMSRTRLPSPP
APSPAFSADSFSDLLRQFDPSLFNTSLFDSLPPFGAHHTEAATGEWDEVQSGLRAADAPPPTMRVAVTAARPPRAKP
APRRRAAQPSDASPAAQVDLRTLGYSQQQQEKIKPKVRSTVAQHHEALVGHGFTHAHIVALSQHPAALGTVAVKYQD
MIAALPEATHEAIVGVGKQWSGARALEALLTVAGELRGPPLQLDTGQLLKIAKRGGVTAVEAVHAWRNALTGAPLNL
TPQQVVAIASNGGGKQALETVQRLLPVLCQAHGLTPQQVVAIASNIGGKQALETVQRLLPVLCQAHGLTPQQVVAIA
SNIGGKQALETVQRLLPVLCQAHGLTPQQVVAIASNNGGKQALETVQRLLPVLCQAHGLTPEQVVAIASNNGGKQAL
ETVQALLPVLCQAHGLTPEQVVAIASNNGGKQALETVQRLLPVLCQAHGLTPQQVVAIASHDGGKQALETVQRLLPV
LCQAHGLTPQQVVAIASNGGGKQALETVQRLLPVLCQAHGLTPQQVVAIASNIGGKQALETVQRLLPVLCQAHGLTP
QQVVAIASNGGGKQALETVQRLLPVLCQAHGLTPEQVVAIASNIGGKQALETVQRLLPVLCQAHGLTPEQVVAIASN
IGGKQALETVQRLLPVLCQAHGLTPEQVVAIASHDGGKQALETVQRLLPVLCQAHGLTPEQVVAIASNIGGKQALET
VQRLLPVLCQAHGLTPQQVVAIASNNGGKQALETVQRLLPVLCQAQGLTPEQVVAIASHDGGKQALETVQRLLPVLC
QAHGLTPQQVVAIASNIGGRPALESIVAQLSRPDPALAALTNDHLVALACLGGRPALDAVKKGLPHAPALIKRTNRR
IPERTSHRVAKQLVKSELEEKKSELRHKLKYVPHEYIELIEIARNSTQDRILEMKVMEFFMKVYGYRGKHLGGSRKP
DGAIYTVGSPIDYGVIVDTKAYSGGYNLPIGQADEMQRYVEENQTRNKHINPNEWWKVYPSSVTEFKFLFVSGHFKG
NYKAQLTRLNHITNCNGAVLSVEELLIGGEMIKAGTLTLEEVRRKFNNGEINF
SEQ ID NO: 10. StALS CDNA
ATGGCGGCTGCTGCCTCACCATCTCCATGTTTCTCCAAAACCCTACCTCCATCTTCCTCCAAATCTTCCACCATTCT
TCCTAGATCTACCTTCCCTTTCCACAATCACCCTCAAAAAGCCTCACCCCTTCATCTCACCCACACCCATCATCATC
GTCGTGGTTTCGCCGTTTCCAATGTCGTCATATCCACTACCACCCATAACGACGTTTCTGAACCTGAAACATTCGTT
TCCCGTTTCGCCCCTGACGAACCCAGAAAGGGTTGTGATGTTCTTGTGGAGGCACTTGAAAGGGAGGGGGTTACGGA
TGTATTTGCGTACCCAGGAGGTGCTTCTATGGAGATTCATCAGGCTTTGACACGTTCGAATATTATTCGTAATGTGC
TGCCACGTCATGAGCAAGGTGGTGTGTTTGCTGCAGAGGGTTACGCACGGGCGACTGGGTTCCCTGGTGTTTGCATT
GCTACCTCTGGTCCGGGAGCTACGAATCTTGTTAGTGGTCTTGCGGATGCTTTGTTGGATAGTATTCCGATTGTTGC
TATTACGGGTCAAGTGCCGAGGAGGATGATTGGTACTGATGCGTTTCAGGAAACGCCTATTGTTGAGGTAACGAGAT
CTATTACGAAGCATAATTATCTTGTTATGGATGTAGAGGATATTCCTAGGGTTGTTCGTGAAGCGTTTTTTCTAGCG
AAATCGGGACGGCCTGGGCCGGTTTTGATTGATGTACCTAAGGATATTCAGCAACAATTGGTGATACCTAATTGGGA
TCAGCCAATGAGGTTGCCTGGTTACATGTCTAGGTTACCTAAATTGCCTAATGAGATGCTTTTGGAACAAATTATTA
GGCTGATTTCGGAGTCGAAGAAGCCTGTTTTGTATGTGGGTGGTGGGTGTTTGCAATCAAGTGAGGAGCTGAGACGA
TTTGTGGAGCTTACGGGTATTCCTGTGGCGAGTACTTTGATGGGTCTTGGAGCTTTTCCAACTGGGGATGAGCTTTC
CCTTCAAATGTTGGGTATGCATGGGACTGTGTATGCTAATTATGCTGTGGATGGTAGTGATTTGTTGCTTGCATTTG
GGGTGAGGTTTGATGATCGAGTTACTGGTAAATTGGAAGCTTTTGCTAGCCGAGCGAAAATTGTCCACATTGATATT
GATTCGGCTGAGATTGGAAAGAACAAGCAACCTCATGTTTCCATTTGTGCAGATATCAAGTTGGCATTACAGGGTTT
GAATTCCATATTGGAGGGTAAAGAAGGTAAGCTGAAGTTGGACTTTTCTGCTTGGAGACAGGAGTTAACGGAACAGA
AGGTGAAGTACCCATTGAGTTTTAAGACTTTTGGTGAAGCCATCCCTCCACAATATGCTATTCAGGTTCTTGATGAG
TTAACTAACGGAAATGCCATTATTAGTACTGGTGTGGGGCAACACCAGATGTGGGCTGCCCAATACTATAAGTACAA
AAAGCCACACCAATGGTTGACATCTGGTGGATTAGGAGCAATGGGATTTGGTTTGCCTGCTGCAATAGGTGCGGCTG
TTGGAAGACCGGGTGAGATTGTGGTTGACATTGATGGTGACGGGAGTTTTATCATGAATGTGCAGGAGTTAGCAACA
ATTAAGGTGGAGAATCTCCCAGTTAAGATTATGTTGCTGAATAATCAACACTTGGGAATGGTGGTTCAATGGGAGGA
TCGATTCTATAAGGCTAACAGAGCACACACTTACTTGGGTGATCCTGCTAATGAGGAAGAGATCTTCCCTAATATGT
TGAAATTCGCAGAGGCTTGTGGCGTACCTGCTGCAAGAGTGTCACACAGGGATGATCTTAGAGCTGCCATTCAAAAG
ATGTTAGACACTCCTGGGCCATACTTGTTGGATGTGATTGTACCTCATCAGGAGCACGTTCTACCTATGATTCCCAG
TGGCGGTGCTTTCAAAGATGTGATCACAGAGGGTGATGGGAGACGTTCATATTGA
SEQ ID NO: 11. StALS AMINO ACID
MAAAASPSPCFSKTLPPSSSKSSTILPRSTFPFHNHPQKASPLHLTHTHHHRRGFAVSNVVISTTTHNDVSEPETFV
SRFAPDEPRKGCDVLVEALEREGVTDVFAYPGGASMEIHQALTRSNIIRNVLPRHEQGGVFAAEGYARATGFPGVCI
ATSGPGATNLVSGLADALLDSIPIVAITGQVPRRMIGTDAFQETPIVEVTRSITKHNYLVMDVEDIPRVVREAFFLA
KSGRPGPVLIDVPKDIQQQLVIPNWDQPMRLPGYMSRLPKLPNEMLLEQIIRLISESKKPVLYVGGGCLQSSEELRR
FVELTGIPVASTLMGLGAFPTGDELSLQMLGMHGTVYANYAVDGSDLLLAFGVRFDDRVTGKLEAFASRAKIVHIDI
DSAEIGKNKQPHVSICADIKLALQGLNSILEGKEGKLKLDFSAWRQELTEQKVKYPLSFKTFGEAIPPQYAIQVLDE
LTNGNAIISTGVGQHQMWAAQYYKYKKPHQWLTSGGLGAMGFGLPAAIGAAVGRPGEIVVDIDGDGSFIMNVQELAT
IKVENLPVKIMLLNNQHLGMVVQWEDRFYKANRAHTYLGDPANEEEIFPNMLKFAEACGVPAARVSHRDDLRAAIQK
MLDTPGPYLLDVIVPHQEHVLPMIPSGGAFKDVITEGDGRRSY
SEQ ID NO: 12. mStALS CDNA (G804A-SILENT MUTATION TO GET RID OF BSTEII SITES,
TG1687/1688CT-W563L, G1925T-S642I)
ATGGCGGCTGCTGCCTCACCATCTCCATGTTTCTCCAAAACCCTACCTCCATCTTCCTCCAAATCTTCCACCATTCT
TCCTAGATCTACCTTCCCTTTCCACAATCACCCTCAAAAAGCCTCACCCCTTCATCTCACCCACACCCATCATCATC
GTCGTGGTTTCGCCGTTTCCAATGTCGTCATATCCACTACCACCCATAACGACGTTTCTGAACCTGAAACATTCGTT
TCCCGTTTCGCCCCTGACGAACCCAGAAAGGGTTGTGATGTTCTTGTGGAGGCACTTGAAAGGGAGGGGGTTACGGA
TGTATTTGCGTACCCAGGAGGTGCTTCTATGGAGATTCATCAGGCTTTGACACGTTCGAATATTATTCGTAATGTGC
TGCCACGTCATGAGCAAGGTGGTGTGTTTGCTGCAGAGGGTTACGCACGGGCGACTGGGTTCCCTGGTGTTTGCATT
GCTACCTCTGGTCCGGGAGCTACGAATCTTGTTAGTGGTCTTGCGGATGCTTTGTTGGATAGTATTCCGATTGTTGC
TATTACGGGTCAAGTGCCGAGGAGGATGATTGGTACTGATGCGTTTCAGGAAACGCCTATTGTTGAGGTAACGAGAT
CTATTACGAAGCATAATTATCTTGTTATGGATGTAGAGGATATTCCTAGGGTTGTTCGTGAAGCGTTTTTTCTAGCG
AAATCGGGACGGCCTGGGCCGGTTTTGATTGATGTACCTAAGGATATTCAGCAACAATTGGTGATACCTAATTGGGA
TCAGCCAATGAGGTTGCCTGGTTACATGTCTAGATTACCTAAATTGCCTAATGAGATGCTTTTGGAACAAATTATTA
GGCTGATTTCGGAGTCGAAGAAGCCTGTTTTGTATGTGGGTGGTGGGTGTTTGCAATCAAGTGAGGAGCTGAGACGA
TTTGTGGAGCTTACGGGTATTCCTGTGGCGAGTACTTTGATGGGTCTTGGAGCTTTTCCAACTGGGGATGAGCTTTC
CCTTCAAATGTTGGGTATGCATGGGACTGTGTATGCTAATTATGCTGTGGATGGTAGTGATTTGTTGCTTGCATTTG
GGGTGAGGTTTGATGATCGAGTTACTGGTAAATTGGAAGCTTTTGCTAGCCGAGCGAAAATTGTCCACATTGATATT
GATTCGGCTGAGATTGGAAAGAACAAGCAACCTCATGTTTCCATTTGTGCAGATATCAAGTTGGCATTACAGGGTTT
GAATTCCATATTGGAGGGTAAAGAAGGTAAGCTGAAGTTGGACTTTTCTGCTTGGAGACAGGAGTTAACGGAACAGA
AGGTGAAGTACCCATTGAGTTTTAAGACTTTTGGTGAAGCCATCCCTCCACAATATGCTATTCAGGTTCTTGATGAG
TTAACTAACGGAAATGCCATTATTAGTACTGGTGTGGGGCAACACCAGATGTGGGCTGCCCAATACTATAAGTACAA
AAAGCCACACCAATGGTTGACATCTGGTGGATTAGGAGCAATGGGATTTGGTTTGCCTGCTGCAATAGGTGCGGCTG
TTGGAAGACCGGGTGAGATTGTGGTTGACATTGATGGTGACGGGAGTTTTATCATGAATGTGCAGGAGTTAGCAACA
ATTAAGGTGGAGAATCTCCCAGTTAAGATTATGTTGCTGAATAATCAACACTTGGGAATGGTGGTTCAACTGGAGGA
TCGATTCTATAAGGCTAACAGAGCACACACTTACTTGGGTGATCCTGCTAATGAGGAAGAGATCTTCCCTAATATGT
TGAAATTCGCAGAGGCTTGTGGCGTACCTGCTGCAAGAGTGTCACACAGGGATGATCTTAGAGCTGCCATTCAAAAG
ATGTTAGACACTCCTGGGCCATACTTGTTGGATGTGATTGTACCTCATCAGGAGCACGTTCTACCTATGATTCCCAT
TGGCGGTGCTTTCAAAGATGTGATCACAGAGGGTGATGGGAGACGTTCATATTGA
SEQ ID NO: 13. mStALS AMINO ACID (MUTATIONS W563L AND S642I)
MAAAASPSPCFSKTLPPSSSKSSTILPRSTFPFHNHPQKASPLHLTHTHHHRRGFAVSNVVISTTTHNDVSEPETFV
SRFAPDEPRKGCDVLVEALEREGVTDVFAYPGGASMEIHQALTRSNIIRNVLPRHEQGGVFAAEGYARATGFPGVCI
ATSGPGATNLVSGLADALLDSIPIVAITGQVPRRMIGTDAFQETPIVEVTRSITKHNYLVMDVEDIPRVVREAFFLA
KSGRPGPVLIDVPKDIQQQLVIPNWDQPMRLPGYMSRLPKLPNEMLLEQIIRLISESKKPVLYVGGGCLQSSEELRR
FVELTGIPVASTLMGLGAFPTGDELSLQMLGMHGTVYANYAVDGSDLLLAFGVRFDDRVTGKLEAFASRAKIVHIDI
DSAEIGKNKQPHVSICADIKLALQGLNSILEGKEGKLKLDFSAWRQELTEQKVKYPLSFKTFGEAIPPQYAIQVLDE
LTNGNAIISTGVGQHQMWAAQYYKYKKPHQWLTSGGLGAMGFGLPAAIGAAVGRPGEIVVDIDGDGSFIMNVQELAT
IKVENLPVKIMLLNNQHLGMVVQLEDRFYKANRAHTYLGDPANEEEIFPNMLKFAEACGVPAARVSHRDDLRAAIQK
MLDTPGPYLLDVIVPHQEHVLPMIPIGGAFKDVITEGDGRRSY
SEQ ID NO: 14. PSIM2168 PDNA (LB TO RB)
TGGCAGGATATATACCGGTGTAAACGAAGTGTGTGTGGTTGATCCAAAATCTATCGTACCTTTAGAAAGTGTAGCTA
TGAAGGATAGTCTCACTTATGAAGAACTACCTATTGAGATTCTTGATCGTCAGGTCCGAAGGTTGAGAAAAATAGAA
GTCGCTTCAGTTACGGCTTTGTGGAGGAGTAAGGGTACCAGTTATACACCCTACATTCTACTCGAGTCATTATGATG
ATGTCTCACGACCAAATCAAATCAAAGTTAAATAAATATCGAACCGAACGCCCACTCTGTATGAGTATGGCAAAAGA
TTTTGAGAGAATCAAGTTGCATAAAAGCCTAATTTTCATGGAACATACAAATTGAGTCTCATAATAGCCCAAACTCA
CAGCCATGAACCCAAATTGGGTAAAGTTTTGCAAGACGTTCATCAAACAGTTAGGAAACATAAAATGGCGCTAGATA
TATAATAAATTTTTTTAACATATGGTGTGATTGATAGTTATATACTAAAGATGTTTGCTTAGTTACGTAATTTTTTC
AAAAAAAAAAGGTACATTATCAATCATCAGTCACAAAATATTAAAAGTTACTGTTTGTTTTTTAAATTCCATGTCGA
ATTTAATTGAATGACACTTAAATTGGGACGAACGGTGTAATTTCTTTTGACTATTCTACTAGTATCTATCCACAGCA
CGTGTTGTTCCTTTCTTCTTTCGTTTTTCATTTACTTGACATTATTAGGAGACTTGGCCCTGAACTCCAACTATTCT
AAGCTGACCTTTCTTTTCCTTTACCAATTATCTTCTTCTTTCTAATTTCGTTTTACGCGTAGTACTGCCTGAATTTT
CTGACTTTCAACGTTTGTTATTCATGCTTGAAAACGAAATACCAGCTAACAAAAGATGAATTATTGTGTTTACAAGA
CTTGGGCCGTTGACTCTTACTTTCCCTTCCTCATCCTCACATTTAGAAAAAAGAAATTTAACGAAAAATTAAAGGAG
ATGGCTGAAATTCTTCTCACAGCAGTCATCAATAAATCAATAGAAATAGCTGGAAATGTACTCTTTCAAGAAGGTAC
GCGTTTATATTGGTTGAAAGAGGACATCGATTGGCTCCAGAGAGAAATGAGACACATTCGATCATATGTAGACAATG
CAAAGGCAAAGGAAGTTGGAGGCGATTCAAGGGTGAAAAACTTATTAAAAGATATTCAACAACTGGCAGGTGATGTG
GAGGATCTATTAGATGAGTTTCTTCCAAAAATTCAACAATCCAATAAGTTCATTTGTTGCCTTAAGACGGTTTCTTT
TGCCGATGAGTTTGCTATGGAGATTGAGAAGATAAAAAGAAGAGTTGCTGATATTGACCGTGTAAGGACAACTTACA
GCATCACAGATACAAGTAACAATAATGATGATTGCATTCCATTGGACCGGAGAAGATTGTTCCTTCATGCTGATGAA
ACAGAGGTCATCGGTCTGGAAGATGACTTCAATACACTACAAGCCAAATTACTTGATCATGATTTGCCTTATGGAGT
TGTTTCAATAGTTGGCATGCCCGGTTTGGGAAAAACAACTCTTGCCAAGAAACTTTATAGGCATGTCTGTCATCAAT
TTGAGTGTTCGGGACTGGTCTATGTTTCACAACAGCCAAGGGCGGGAGAAATCTTACATGACATAGCCAAACAAGTT
GGACTGACGGAAGAGGAAAGGAAAGAAAACTTGGAGAACAACCTACGATCACTCTTGAAAATAAAAAGGTATGTTAT
TCTCTTAGATGACATTTGGGATGTTGAAATTTGGGATGATCTAAAACTTGTCCTTCCTGAATGTGATTCAAAAATTG
GCAGTAGGATAATTATAACCTCTCGAAATAGTAATGTAGGCAGATACATAGGAGGGGATTTCTCAATCCACGTGTTG
CAACCCCTAGATTCAGAGAAAAGCTTTGAACTCTTTACCAAGAAAATCTTTAATTTTGTTAATGATAATTGGGCCAA
TGCTTCACCAGACTTGGTAAATATTGGTAGATGTATAGTTGAGAGATGTGGAGGTATACCGCTAGCAATTGTGGTGA
CTGCAGGCATGTTAAGGGCAAGAGGAAGAACAGAACATGCATGGAACAGAGTACTTGAGAGTATGGCTCATAAAATT
CAAGATGGATGTGGTAAGGTATTGGCTCTGAGTTACAATGATTTGCCCATTGCATTAAGGCCATGTTTCTTGTACTT
TGGTCTTTACCCCGAGGACCATGAAATTCGTGCTTTTGATTTGACAAATATGTGGATTGCTGAGAAGCTGATAGTTG
TAAATACTGGCAATGGGCGAGAGGCTGAAAGTTTGGCGGATGATGTCCTAAATGATTTGGTTTCAAGAAACTTGATT
CAAGTTGCCAAAAGGACATATGATGGAAGAATTTCAAGTTGTCGCATACATGACTTGTTACATAGTTTGTGTGTGGA
CTTGGCTAAGGAAAGTAACTTCTTTCACACGGAGCACAATGCATTTGGTGATCCTAGCAATGTTGCTAGGGTGCGAA
GGATTACATTCTACTCTGATGATAATGCCATGAATGAGTTCTTCCATTTAAATCCTAAGCCTATGAAGCTTCGTTCA
CTTTTCTGTTTCACAAAAGACCGTTGCATATTTTCTCAAATGGCTCATCTTAACTTCAAATTATTGCAAGTGTTGGT
TGTAGTCATGTCTCAAAAGGGTTATCAGCATGTTACTTTCCCCAAAAAAATTGGGAACATGAGTTGCCTACGTTATG
TGCGATTGGAGGGGGCAATTAGAGTAAAATTGCCAAATAGTATTGTCAAGCTCAAATGTCTAGAGACCCTGGATATA
TTTCATAGCTCTAGTAAACTTCCTTTTGGTGTTTGGGAGTCTAAAATATTGAGACATCTTTGTTACACAGAAGAATG
TTACTGTGTCTCTTTTGCAAGTCCATTTTGCCGAATCATGCCTCCTAATAATCTACAAACTTTGATGTGGGTGGATG
ATAAATTTTGTGAACCAAGATTGTTGCACCGATTGATAAATTTAAGAACATTGTGTATAATGGATGTATCCGGTTCT
ACCATTAAGATATTATCAGCATTGAGCCCTGTGCCTAGAGCGTTGGAGGTTCTGAAGCTCAGATTTTTCAAGAACAC
GAGTGAGCAAATAAACTTGTCGTCCCATCCAAATATTGTCGAGTTGGGTTTGGTTGGTTTCTCAGCAATGCTCTTGA
ACATTGAAGCATTCCCTCCAAATCTTGTCAAGCTTAATCTTGTCGGCTTGATGGTAGACGGTCATCTATTGGCAGTG
CTTAAGAAATTGCCCAAATTAAGGATACTTATATTGCTTTGGTGCAGACATGATGCAGAAAAAATGGATCTCTCTGG
TGATAGCTTTCCGCAACTTGAAGTTTTGTATATTGAGGATGCACAAGGGTTGTCTGAAGTAACGTGCATGGATGATA
TGAGTATGCCTAAATTGAAAAAGCTATTTCTTGTACAAGGCCCAAACATTTCCCCAATTAGTCTCAGGGTCTCGGAA
CGGCTTGCAAAGTTGAGAATATCACAGGTACTATAAATAATTATTTACGTTTAATATCCATGATTTTTTTAAATTTG
TATTTAGTTCATCAACTAAATATTCCATGTCTAATAAATTGCAGGGATGCCTTTGAAAATGATTCTGTGTTGGAGAG
AATCTTCTGATGCCTGTTGGTATTATAATACTAATAATAAGAGAAAAAGTTTGATTACTGTTTCAAGTTAATTGCTT
GTGATTTGTAAAAACAAATTACTTTTATATTTCTCTTTGTTTTATTTTATGTTTATTTATCTTTAATTAATGGAGTA
ATAAAATAAAAATCTTATTTTCAATAGAAAAAAGTAGACCTTATTTGTGGTGCATGTATGGTATCTTTTTGAAATTT
TTGATATATTTGCTCTTTGATTCGAATTTCTTGCTTATATGATGATTTGCATAAATATAAAATATTATACAAATACC
TATGGGTTGGAAAATATAGAAATATGCCAATCAAATGTATACAAAAATCATTAATAGATAGAATCGTAAAAGATATA
CAAATGAGAAATGCTTGACTAAGAAGCTTCGTGCAACCTCTCACACTGAGCACAATGCATTTGGTGATCTCGGCACT
ATTGCTGTTACTTGTAAGACTACGTTCCCCAATAAGTCTTTCCAAACGGCTTGCAAAGCTGAGAATATGAAAATCTC
ATAGGTTAGTTTGCTGCGTTAATTATTTACATTTAATATGCTCGATAAGGTGATTTTAAAAAAATTTGTACTAGTTA
ATTCATGAACTAAATATTTCATTTAATACTCCATAATTCTGAATATGGAAAATAAATAATATTTAATAACAAGAATA
AAATGATAAATTATTCATTGATTTTATAAATTGGATAAATATTATTAAATATTCTTAAATAATATAATGAACAAGTG
AAGATGAACGGAGGGAGTATGAAGCCTCTTTTCAAAGGGGCCCCAAGTGTCTGAGACAACCAAAACTGAAAGTGGGA
AACCAAACTCTAAGTCAAAGACTTTATATACAAAATGGTATAAATATAATTATTTAATTTACTATCGGGTTATCGAT
TAACCCGTTAAGAAAAAACTTCAAACCGTTAAGAACCGATAACCCGATAACAAAAAAAATCTAAATCGTTATCAAAA
CCGCTAAACTAATAACCCAATATTGATAAACCAATAACTTTTTTTATTCGGGTTATCGGTTTCAGTTCTGTTTGGAA
CAATCCTAGTGTCCTAATTATTGTTTTGAGAACCAAGAAAACAAAAACTTACGTCGCAAATATTTCAGTAAATACTT
GTATATCTCAGTGATAATTGATTTCCAACATGTATAATTATCATTTACGTAATAATAGATGGTTTCCGAAACTTACG
CTTCCCTTTTTTCTTTTGCAGTCGTATGGAATAAAAGTTGGATATGGAGGCATTCCCGGGCCTTCAGGTGGAAGAGA
CGGAGCTGCTTCACAAGGAGGGGGTTGTTGTACTTGAAAATGGGCATTTATTGTTCGCAAACCTATCATGTTCCTAT
GGTTGTTTATTTGTAGTTTGGTGTTCTTAATATCGAGTGTTCTTTAGTTTGTTCCTTTTAATGAAAGGATAATATCT
GTGCAAAAATAAGTAAATTCGGTACATAAAGACATTTTTTTTTGCATTTTCTGTTTATGGAGTTGTCAAATGTGAAT
TTATTTCATAGCATGTGAGTTTCCTCTCCTTTTTCATGTGCCCTTGGGCCTTGCATGTTTCTTGCACCGCAGTGTGC
CAGGGCTGTCGGCAGATGGACATAAATGGCACACCGCTCGGCTCGTGGAAAGAGTATGGTCAGTTTCATTGATAAGT
ATTTACTCGTATTCGGTGTTTACATCAAGTTAATATGTTCAAACACATGTGATATCATACATCCATTAGTTAAGTAT
AAATGCCAACTTTTTACTTGAATCGCCGAATAAATTTACTTACGTCCAATATTTAGTTTTGTGTGTCAAACATATCA
TGCACTATTTGATTAAGAATAAATAAACGATGTGTAATTTGAAAACCAATTAGAAAAGAAGTATGACGGGATTGATG
TTCTGTGAAATCACTGGTAAATTGGACGGACGATGAAATTTGATCGTCCATTTAAGCATAGCAACATGGGTCTTTAG
TCATCATCATTATGTTATAATTATTTTCTTGAAACTTGATACACCAACTTTCATTGGGAAAGTGACAGCATAGTATA
AACTATAATATCAATTCTGGCAATTTCGAATTATTCCAAATCTCTTTTGTCATTTCATTTCCTCCCCTATGTCTGCA
AGTACCAATTATTTAAGTACAAAAAATCTTGATTAAACAATTTATTTTCTCACTAATAATCACATTTAATCATCAAC
GGTTCATACACGTCTGTCACTCTTTTTTTATTCTCTCAAGCGCATGTGATCATACCAATTATTTAAATACAAAAAAT
CTTGATTAAACAATTCAGTTTCTCACTAATAATCACATTTAATCATCAACGGTTCATACACATCCGTCACTCTTTTT
TTATTCTCTCAAGCGCATGTGATCATACCAATTATTTAAATACAAAAAATCTTGATTAAACAATTCATTTTCTCACT
AATAATCACATTTAATCATCAACGGTTTATACACGTCCGCCACTCTTTTTTTATTCTCTCAAGCGTATGTGATCATA
TCTAACTCTCGTGCAAACAAGTGAAATGACGTTCACTAATAAATAATCTTTTGAATACTTTGTTCAGTTTAATTTAT
TTAATTTGATAAGAATTTTTTTATTATTGAATTTTTATTGTTTTAAATTAAAAATAAGTTAAATATATCAAAATATC
TTTTAATTTTATTTTTGAAAAATAACGTAGTTCAAACAAATTAAAATTGAGTAACTGTTTTTCGAAAAATAATGATT
CTAATAGTATATTCTTTTTCATCATTAGATATTTTTTTTAAGCTAAGTACAAAAGTCATATTTCAATCCCCAAAATA
GCCTCAATCACAAGAAATGCTTAAATCCCCAAAATACCCTCAATCACAAGACGTGTGTACCAATCATACCTATGGTC
CTCTCGTAAATTCCGACAAAATCAGGTCTATAAAGTTACCCTTGATATCAGTATTATAAAACTAAAAATCTCAGCTG
TAATTCAAGTGCAATCACACTCTACCACACACTCTCTAGTAGAGAGATCAGTTGATAACAAGCTTGTTAACGGATCC
ATAATTGTAACTGATTTATTCTTGAATAACAACTTCAATGAAATCAAGCAACAAAGCTGATTTCAACATAAAAAAAC
AGAACAAGAAAACAAAAACAGAGCATCATCCATCAAAGTGTAATCTCAGCAGATTCAATAGAGACTACAAGATTTTG
CACTTGTACATAATCATCAGTGTCACCGGTATAAAGCATCATGATCTGACCATCGGGTAGGATGGTAGCGGACCCAG
TCCAGACACCGTTAATATCGTACCATTGATCAGGAACCATGGCAAAAGGCAAGTAGAGCCAGTGGATCAAGTCCTTG
GATACGGCATGGCCCCATGTGATATTTCCCCAAATAGCTGAATCTGGATTGTATTGATAAAAAAGATGATACCATCC
CTTGTGGTACAATGGACCATTAGGATCGTTCATCCAATTTTTTTGAGGTTGAAAATGGTAAGCAGTTCTTTGCCAGC
TAAGCATAGCATTGGACCACGCATAAGAAACGTGACTAGCATTGACGACATCTCGAAAAGTCTTATCGGAGACTCCC
TGAGAAACACCTCTTGACGGCGGCGCCGGCGAACGGGAGTTACTCTGCAAGTCCGGTGACTGGTTGTTGAGGATCGG
AAAGAAGGCTACAGAAAGCAAAAGGAAAGAGGAGAGGAAAATGCCGGAGATGATTTTAAGGGACTTCCGGTGGCCGG
AATCGGGTTGATCCGGGAGGAATGTGTAATGGGAGGCGGAGTTTTCCGGGTCATAACTGGAATGGTACTGCGTGGCC
ATACTCGTGCCTAAAATGGCGAATAAGTAGAGTATAACACTACATATTCTCCCTCTCTTCCCTTTCTTGATGGGACA
TCGGTGAAATAACCTTCAAATGAAAAAAAGAATGAAGAAGATATGGCTTGATGAAGAACTCTTTATCCAGAAATGGT
ACTCTAGCTTCTAAGCCCCACGCGGATGTAGCCTTGTTTGCTCTTAAACAGTCATACTGGTGAAGCGCTTTTATCTT
GCGACATGTTTCCGTGTGGAACTCTTCCTTGTTTGGAGCCTTGTGGAAGTACAAGTAGCCACCAAAAATTTCGTCAG
CACCTTCCCCTGATATGACCATCTTCACTCCTAGTGATTTAATCTTACGTGACATAAGGAACATAGGAGTGCTGGCT
CTTATTGTTGTTACATCATACGTCTCGATATGATATATAACATCTTCAATAGCATCAATCCCGTCCTGAACAGTAAA
GTGAAACTCGTGGTGAACGGTTCCTAAAAAGTCAGCAACTTCTTTTGCAGCCTTGAGATCTGGTGAGCCCTCGAGAA
CATTTTGAAGTTTTCCCTCCGGGGCACTTGTACTCTAGCAAGAACGGAGGGCTTAGGAGATGGTACAATCCCGCTTG
GTTCTCTGAAGCAATTCCTTCCACTCCTTATGACACTTTGGTTCTGAGGCGTGCCTTCGAAAATGCTGTTATCAAAC
GGTTGATGACTGATGTCCCCTTTGGCGTTCTGCTCTCGGGGGGACTTGATTCGTCTTTGGTTGCTTCTGTCACTACT
CGATACTTGGCTGGAACAAAAGCTGCTAAGCAATGGGGAGCACAACTTCATTCCTTCTGTGTTGGTCTCGAGGGCTC
ACCAGATCTCAAGGCTGCAAAAGAAGTTGCTGACTTTTTAGGAACCGTTCACCACGAGTTTCACTTTACTGTTCAGG
ACGGGATTGATGCTATTGAAGATGTTATATATCATATCGAGACGTATGATGTAACAACAATAAGAGCCAGCACTCCT
ATGTTCCTTATGTCACGTAAGATTAAATCACTAGGAGTGAAGATGGTCATATCAGGGGAAGGTGCTGACGAAATTTT
TGGTGGCTACTTGTACTTCCACAAGGCTCCAAACAAGGAAGAGTTCCACACGGAAACATGTCGCAAGATAAAAGCGC
TTCACCAGTATGACTGTTTAAGAGCAAACAAGGCTACATCCGCGTGGGGCTTAGAAGCTAGAGTACCATTTCTGGAT
AAAGAGTTCTTCATCAAGCCATATCTTCTTCATTCTTTTTTTCATTTGAAGGTTATTTCACCGATGTCCCATCAAGA
AAGGGAAGAGAGGGAGAATATGTAGTGTTATACTCTACTTATTCGCCATTTTAGGCACGAGTATGGCCACGCAGTAC
CATTCCAGTTATGACCCGGAAAACTCCGCCTCCCATTACACATTCCTCCCGGATCAACCCGATTCCGGCCACCGGAA
GTCCCTTAAAATCATCTCCGGCATTTTCCTCTCCTCTTTCCTTTTGCTTTCTGTAGCCTTCTTTCCGATCCTCAACA
ACCAGTCACCGGACTTGCAGAGTAACTCCCGTTCGCCGGCGCCGCCGTCAAGAGGTGTTTCTCAGGGAGTCTCCGAT
AAGACTTTTCGAGATGTCGTCAATGCTAGTCACGTTTCTTATGCGTGGTCCAATGCTATGCTTAGCTGGCAAAGAAC
TGCTTACCATTTTCAACCTCAAAAAAATTGGATGAACGATCCTAATGGTCCATTGTACCACAAGGGATGGTATCATC
TTTTTTATCAATACAATCCAGATTCAGCTATTTGGGGAAATATCACATGGGGCCATGCCGTATCCAAGGACTTGATC
CACTGGCTCTACTTGCCTTTTGCCATGGTTCCTGATCAATGGTACGATATTAACGGTGTCTGGACTGGGTCCGCTAC
CATCCTACCCGATGGTCAGATCATGATGCTTTATACCGGTGACACTGATGATTATGTACAAGTGCAAAATCTTGTAG
TCTCTATTGAATCTGCTGAGATTACACTTTGATGGATGATGCTCTGTTTTTGTTTTCTTGTTCTGTTTTTTTATGTT
GAAATCAGCTTTGTTGCTTGATTTCATTGAAGTTGTTATTCAAGAATAAATCAGTTACAATTATACTAGTCCCTAGA
CTTGTCCATCTTCTGGATTGGCCAACTTAATTAATGTATGAAATAAAAGGATGCACACATAGTGACATGCTAATCAC
TATAATGTGGGCATCAAAGTTGTGTGTTATGTGTAATTACTAATTATCTGAATAAGAGAAAGAGATCATCCATATTT
CTTATCCTAAATGAATGTCACGTGTCTTTATAATTCTTTGATGAACCAGATGCATTTTATTAACCAATTCCATATAC
GAGCTCCCTATTTTTTTACTATATTATACTCAACCCAATGAGCATAAAGACTGTAAAATCTCAAATTCCTGAGAAGC
ATATTTATCGATCCCACAGACTTGATAGTTCCATAATCCATACGCTGCAGCCAAATTGCTAGTGTGTTGAACATTTA
ACACGTAGAGAACTAGAAAAGATATAAAACTAAGATTGATATCCAAAATAGACGAGAACAATAAGCAAAAACTCTTA
GTTTTGAAATAAATCAACAATCCCGAGGGTTGTCACATACATCAAAAACGAAAATCCATATAGCAAAAAAAACTCTA
AATTACCGTTCGACAAAAAGAGAAAACTGATAGGACATTTGCTAAACATTAAAATCAATATGAACGTCTCCCATCAC
CCTCTGTGATCACATCTTTGAAAGCACCGCCAATGGGAATCATAGGTAGAACGTGCTCCTGATGAGGTACAATCACA
TCCAACAAGTATGGCCCAGGAGTGTCTAACATCTTTTGAATGGCAGCTCTAAGATCATCCCTGTGTGACACTCTTGC
AGCAGGTACGCCACAAGCCTCTGCGAATTTCAACATATTAGGGAAGATCTCTTCCTCATTAGCAGGATCACCCAAGT
AAGTGTGTGCTCTGTTAGCCTTATAGAATCGATCCTCCAGTTGAACCACCATTCCCAAGTGTTGATTATTCAGCAAC
ATAATCTTAACTGGGAGATTCTCCACCTTAATTGTTGCTAACTCCTGCACATTCATGATAAAACTCCCGTCACCATC
AATGTCAACCACAATCTCACCCGGTCTTCCAACAGCCGCACCTATTGCAGCAGGCAAACCAAATCCCATTGCTCCTA
ATCCACCAGATGTCAACCATTGGTGTGGCTTTTTGTACTTATAGTATTGGGCAGCCCACATCTGGTGTTGCCCCACA
CCAGTACTAATAATGGCATTTCCGTTAGTTAACTCATCAAGAACCTGAATAGCATATTGTGGAGGGATGGCTTCACC
AAAAGTCTTAAAACTCAATGGGTACTTCACCTTCTGTTCCGTTAACTCCTGTCTCCAAGCAGAAAAGTCCAACTTCA
GCTTACCTTCTTTACCCTCCAATATGGAATTCAAACCCTGTAATGCCAACTTGATATCTGCACAAATGGAAACATGA
GGTTGCTTGTTCTTTCCAATCTCAGCCGAATCAATATCAATGTGGACAATTTTCGCTCGGCTAGCAAAAGCTTCCAA
TTTACCAGTAACTCGATCATCAAACCTCACCCCAAATGCAAGCAACAAATCACTACCATCCACAGCATAATTAGCAT
ACACAGTCCCATGCATACCCAACATTTGAAGGGAAAGCTCATCCCCAGTTGGAAAAGCTCCAAGACCCATCAAAGTA
CTCGCCACAGGAATACCCGTAAGCTCCACAAATCGTCTCAGCTCCTCACTTGATTGCAAACACCCACCACCCACATA
CAAAACAGGCTTCTTCGACTCCGAAATCAGCCTAATAATTTGTTCCAAAAGCATCTCATTAGGCAATTTAGGTAATC
TAGACATGTAACCAGGCAACCTCATTGGCTGATCCCAATTAGGTATCACCAATTGTTGCTGAATATCCTTAGGTACA
TCAATCAAAACCGGCCCAGGCCGTCCCGATTTCGCTAGAAAAAACGCTTCACGAACAACCCTAGGAATATCCTCTAC
ATCCATAACAAGATAATTATGCTTCGTAATAGATCTCGTTACCTCAACAATAGGCGTTTCCTGAAACGCATCAGTAC
CAATCATCCTCCTCGGCACTTGACCCGTAATAGCAACAATCGGAATACTATCCAACAAAGCATCCGCAAGACCACTA
ACAAGATTCGTAGCTCCCGGACCAGAGGTAGCAATGCAAACACCAGGGAACCCAGTCGCCCGTGCGTAACCCTCTGC
AGCAAACACACCACCTTGCTCATGACGTGGCAGCACATTACGAATAATATTCGAACGTGTCAAAGCCTGATGAATCT
CCATAGAAGCACCTCCTGGGTACGCAAATACATCCGTAACCCCCTCCCTTTCAAGTGCCTCCACAAGAACATCACAA
CCCTTTCTGGGTTCGTCAGGGGCGAAACGGGAAACGAATGTTTCAGGTTCAGAAACGTCGTTATGGGTGGTAGTGGA
TATGACGACATTGGAAACGGCGAAACCACGACGATGATGATGGGTGTGGGTGAGATGAAGGGGTGAGGCTTTTTGAG
GGTGATTGTGGAAAGGGAAGGTAGATCTAGGAAGAATGGTGGAAGATTTGGAGGAAGATGGAGGTAGGGTTTTGGAG
AAACATGGAGATGGTGAGGCAGCAGCCGCCATACCTCCACGTAGACGGAGCACCAAATGGAGGGTAGACTCCTTCTG
GATGTTGTAATCAGCTAGAGTACGTCCGTCCTCCAACTGCTTTCCGGCGAAGATAAGCCTTTGCTGATCCGGGGGAA
TTCCTTCCTTATCCTGGATCTTAGCCTTAACGTTGTCGATTGTATCAGAACTTTCCACCTCTAGGGTGATAGTCTTT
CCGGTGAGAGTTTTCACAAAGATCTGCATCTGCAAATTCATAAAAACAACAATCAGAACAACGAAAAAAACTCAAAT
TTAGGGCTTTTCACACTCCCTGATTAGCTGATTCGAGAATTAAAACCAAACAATCTAAAACACGGGGAAAAAACTCA
AATTTAGGGCTTTTTCATCAAAACAGCCGGAGAAACTCAAATTTAGGGCTTTTTCATCAAAACAGCCGGAGAAACTC
AAATTTTAGGCCTTCAGAATCAAACCGACGAAAAAACACAAATTCTAGGGCTTTTTCATCAAATCAACCGGAAAAAC
TCAAATTTAAGGCCTTTTTCATCAAACAAATGTAAAAACTCTAATTTTAAACCTTTAGAATCGAATCGACAGAAAAA
AAATCTCAAACTAGGGGCTTTTTCATCAAAACAACGGGGAAAACTCCAATTGTAGGCCTTTTCCATCAAACCAACGT
AAAAAAACTCAAATTTTAGGCCTTTTCCATCAAGACAACCGAAAAAAAACTCAAAATTAAGGGCTATAACAGCACCT
GATTAGCTAATTCGAGAATTGACAGGATATATGGTACTGTAAAC
SEQ ID NO: 15. FMV-TARGET-GUS-NOS
ATTTAGCAGCATTCCAGATTGGGTTCAATCAACAAGGTACGAGCCATATCACTTTATTCAAATTGGTATCGCCAAAA
CCAAGAAGGAACTCCCATCCTCAAAGGTTTGTAAGGAAGAATTCTCAGTCCAAAGCCTCAACAAGGTCAGGGTACAG
AGTCTCCAAACCATTAGCCAAAAGCTACAGGAGATCAATGAAGAATCTTCAATCAAAGTAAACTACTGTTCCAGCAC
ATGCATCATGGTCAGTAAGTTTCAGAAAAAGACATCCACCGAAGACTTAAAGTTAGTGGGCATCTTTGAAAGTAATC
TTGTCAACATCGAGCAGCTGGCTTGTGGGGACCAGACAAAAAAGGAATGGTGCAGAATTGTTAGGCGCACCTACCAA
AAGCATCTTTGCCTTTATTGCAAAGATAAAGCAGATTCCTCTAGTACAAGTGGGGAACAAAATAACGTGGAAAAGAG
CTGTCCTGACAGCCCACTCACTAATGCGTATGACGAACGCAGTGACGACCACAAAAGAATTCCCTCTATATAAGAAG
GCATTCATTCCCATTTGAAGGATCATCAGATACTCAACCAATACTAGTATGTTAAGGGCTATAACAGCACCTGATTA
GCTAATTCGAGAATCTAAACAGACAAAAACCAAAGTCCGTCCTGTAGAAACCCCAACCCGTGAAATCAAAAAACTCG
ACGGCCTGTGGGCATTCAGTCTGGATCGCGAAAACTGTGGAATTGATCAGCGTTGGTGGGAAAGCGCGTTACAAGAA
AGCCGGGCAATTGCTGTGCCAGGCAGTTTTAACGATCAGTTCGCCGATGCAGATATTCGTAATTATGCGGGCAACGT
CTGGTATCAGCGCGAAGTCTTTATACCGAAAGGTTGGGCAGGCCAGCGTATCGTGCTGCGTTTCGATGCGGTCACTC
ATTACGGCAAAGTGTGGGTCAATAATCAGGAAGTGATGGAGCATCAGGGCGGCTATACGCCATTTGAAGCCGATGTC
ACGCCGTATGTTATTGCCGGGAAAAGTGTACGTAAGTTTCTGCTTCTACCTTTGATATATATATAATAATTATCATT
AATTAGTAGTAATATAATATTTCAAATATTTTTTTCAAAATAAAAGAATGTAGTATATAGCAATTGCTTTTCTGTAG
TTTATAAGTGTGTATATTTTAATTTATAACTTTTCTAATATATGACCAAAATTTGTTGATGTGCAGGTATCACCGTT
TGTGTGAACAACGAACTGAACTGGCAGACTATCCCGCCGGGAATGGTGATTACCGACGAAAACGGCAAGAAAAAGCA
GTCTTACTTCCATGATTTCTTTAACTATGCCGGAATCCATCGCAGCGTAATGCTCTACACCACGCCGAACACCTGGG
TGGACGATATCACCGTGGTGACGCATGTCGCGCAAGACTGTAACCACGCGTCTGTTGACTGGCAGGTGGTGGCCAAT
GGTGATGTCAGCGTTGAACTGCGTGATGCGGATCAACAGGTGGTTGCAACTGGACAAGGCACTAGCGGGACTTTGCA
AGTGGTGAATCCGCACCTCTGGCAACCGGGTGAAGGTTATCTCTATGAACTGTGCGTCACAGCCAAAAGCCAGACAG
AGTGTGATATCTACCCGCTTCGCGTCGGCATCCGGTCAGTGGCAGTGAAGGGCGAACAGTTCCTGATTAACCACAAA
CCGTTCTACTTTACTGGCTTTGGTCGTCATGAAGATGCGGACTTGCGTGGCAAAGGATTCGATAACGTGCTGATGGT
GCACGACCACGCATTAATGGACTGGATTGGGGCCAACTCCTACCGTACCTCGCATTACCCTTACGCTGAAGAGATGC
TCGACTGGGCAGATGAACATGGCATCGTGGTGATTGATGAAACTGCTGCTGTCGGCTTTAACCTCTCTTTAGGCATT
GGTTTCGAAGCGGGCAACAAGCCGAAAGAACTGTACAGCGAAGAGGCAGTCAACGGGGAAACTCAGCAAGCGCACTT
ACAGGCGATTAAAGAGCTGATAGCGCGTGACAAAAACCACCCAAGCGTGGTGATGTGGAGTATTGCCAACGAACCGG
ATACCCGTCCGCAAGGTGCACGGGAATATTTCGCGCCACTGGCGGAAGCAACGCGTAAACTCGACCCGACGCGTCCG
ATCACCTGCGTCAATGTAATGTTCTGCGACGCTCACACCGATACCATCAGCGATCTCTTTGATGTGCTGTGCCTGAA
CCGTTATTACGGATGGTATGTCCAAAGCGGCGATTTGGAAACGGCAGAGAAGGTACTGGAAAAAGAACTTCTGGCCT
GGCAGGAGAAACTGCATCAGCCGATTATCATCACCGAATACGGCGTGGATACGTTAGCCGGGCTGCACTCAATGTAC
ACCGACATGTGGAGTGAAGAGTATCAGTGTGCATGGCTGGATATGTATCACCGCGTCTTTGATCGCGTCAGCGCCGT
CGTCGGTGAACAGGTATGGAATTTCGCCGATTTTGCGACCTCGCAAGGCATATTGCGCGTTGGCGGTAACAAGAAAG
GGATCTTCACTCGCGACCGCAAACCGAAGTCGGCGGCTTTTCTGCTGCAAAAACGCTGGACTGGCATGAACTTCGGT
GAAAAACCGCAGCAGGGAGGCAAACAATGAAAGCTTTTGATTTTAATGTTTAGCAAATGTCCTATCAGTTTTCTCTT
TTTGTCGAACGGTAATTTAGAGTTTTTTTTGCTATATGGATTTTCGTTTTTGATGTATGTGACAACCCTCGGGATTG
TTGATTTATTTCAAAACTAAGAGTTTTTGCTTATTGTTCTCGTCTATTTTGGATATCAATCTTAGTTTTATATCTTT
TCTAGTTCTCTACGTGTTAAATGTTCAACACACTAGCAATTTGGCTGCAGCGTATGGATTATGGAACTATCAAGTCT
GTGGGATCGATAAATATGCTTCTCAGGAATTTGAGATTTTACAGTCTTTATGCTCATTGGGTTGAGTATAATATAGT
AAAAAAATAGG
SEQ ID NO: 16
AAGGCATTCATTCCCATTTG
SEQ ID NO: 17
GACCCACACTTTGCCGTAAT

1. A method for stably integrating a desired polynucleotide into a plant genome, comprising
(A) transforming a plant material with a first vector comprising nucleotide sequences encoding TAL proteins designed to recognize a target sequence;
(B) transforming the plant material with a second vector comprising (i) a marker gene that is not operably linked to a promoter (“promoter-free marker cassette”) and which comprises a sequence homologous to the target sequence; and (ii) a desired polynucleotide; and
(C) identifying transformed plant material in which the desired polynucleotide is stably integrated into the plant genome.

2. The method of claim 1, wherein the marker gene is acetolactate synthase (ALS) gene and the transformed plant material is exposed to herbicide.

3. The method of claim 1, wherein the promoter-free marker cassette is stably integrated into the plant genome.

4. A method for the targeted insertion of exogenous DNA into a plant comprising the steps of (i) transforming isolated plant cells with
(A) a first binary vector comprising a promoter-less cassette comprising (a) a right border sequence linked to (b) a partial sequence of the Ubi7 intron 5′-untranslated region; (c) an Ubi7 monomer-encoding sequence fused to a mutated acetolactate synthase (ALS) gene; (d) a desired nucleotide sequence; and (e) a terminator sequence, wherein the desired nucleotide sequence is not operably linked to a promoter and wherein the desired nucleotide sequence is a silencing cassette targeting the asparagine synthase 1 (Asn1) gene, the polyphenol oxidase (Ppo) gene, or the vacuolar invertase (Inv) gene; and
(B) a second binary vector comprising (a) a right border; (b) a forward expression cassette and a reverse expression cassette, each comprising a modified TAL effector operably linked to a strong constitutive promoter, and a terminator sequence; and (c) a sequence encoding isopentenyl transferase (ipt), wherein the modified TAL effector is designed to bind the desired nucleotide sequence within an intron of potato's ubiquitin-7 (Ubi7) gene;
and (ii) culturing the isolated plant cells under conditions that promote growth of plants that express the desired nucleotide sequence; wherein no vector backbone DNA is permanently inserted into the plant genome.

5. The method of claim 4, wherein the modified TAL effector comprises (a) a truncated C-terminal activation domain comprising a Fok1 endonuclease catalytic domain; (b) a codon-optimized target sequence binding domain comprising 16.5 repeat variable diresidues corresponding to the Ubi7 5′-untranslated intron sequence; and (c) an N-terminal region comprising a SV40 nuclear localization sequence.

6. The method of claim 4, wherein the first binary vector further comprises a late blight resistance gene Vnt1 operably linked to its native promoter and terminator sequences.

7. A transformed tuber-bearing plant comprising in its genome an endogenous Ubi7 promoter operably linked to a desired exogenous nucleotide sequence operably linked to an exogenous terminator sequence, wherein the expression of asparagine synthase 1 (Asn1), polyphenol oxidase (Ppo), or vacuolar invertase (Inv) in the transformed plant is down-regulated.

8. The transformed plant of claim 7, wherein the plant further expresses a late blight resistance gene Vnt1.

9. The transformed tuber-bearing plant of claim 7, wherein the tuber-bearing plant is a potato plant having a phenotype characterized by one or more of late blight resistance, black spot bruise tolerance, reduced cold-induced sweetening and reduced asparagine levels in its tubers.

10. A heat-processed product of the transformed tuber-bearing plant of claim 7, wherein the product is a French fry, chip, crisp, potato, dehydrated potato or baked potato and wherein the heat-processed product has a lower level of acrylamide than a heat-processed product of a non-transformed plant of the same species.

11. A modified TAL effector designed to bind to a desired sequence within an intron of potato's ubiquitin-7 (Ubi7) gene comprising (a) a truncated C-terminal activation domain comprising a catalytic domain; (b) a codon-optimized target sequence binding domain; and (c) an N-terminal region comprising a nuclear localization sequence.

12. The modified TAL effector of claim 11, wherein (a) the catalytic domain in the C-terminal activation domain comprises a Fok1 endonuclease; (b) the target sequence binding domain comprises 16.5 repeat variable diresidues corresponding to the Ubi7 5′-untranslated intron sequence; and (c) the nuclear localization sequence in the N-terminal region is a SV40 nuclear localization sequence.

13. A binary vector comprising (a) a right border; (b) a forward expression cassette and a reverse expression cassette, each comprising a modified TAL effector according to claim 11 operably linked to a strong constitutive promoter and a terminator sequence; and (c) a sequence encoding isopentenyl transferase (ipt).

14. A DNA construct comprising a promoter-less cassette comprising (a) a right border sequence linked to (b) a partial Ubi7 5′-untranslated intron sequence; (c) an Ubi7 monomer-encoding sequence fused to a mutated acetolactate synthase (ALS) gene; (d) a desired nucleotide sequence; (e) a terminator sequence; and (f) a left border, wherein the desired nucleotide sequence is a silencing cassette targeting the asparagine synthase 1 (Asn1) gene, the polyphenol oxidase (Ppo) gene, or the vacuolar invertase (Inv) gene, and wherein the desired nucleotide sequence is not operably linked to a promoter.

15. The DNA construct of claim 14, wherein the DNA construct further comprises a late blight resistance gene Vnt1 operably linked to its native promoter and terminator sequences.

16. A kit for targeted insertion of exogenous DNA into a plant comprising:
(A) a first binary vector comprising a promoter-less cassette comprising (a) a right border sequence linked to (b) a partial sequence of the Ubi7 intron 5′-untranslated region; (c) an Ubi7 monomer-encoding sequence fused to a mutated acetolactate synthase (ALS) gene; (d) a desired nucleotide sequence; and (e) a terminator sequence, wherein the desired nucleotide sequence is not operably linked to a promoter, wherein the desired nucleotide sequence is a silencing cassette targeting the asparagine synthase 1 (Asn1) gene, the polyphenol oxidase (Ppo) gene, or the vacuolar invertase (Inv) gene; and
(B) a second binary vector comprising (a) a right border; (b) a forward expression cassette and a reverse expression cassette, each comprising a modified TAL effector operably linked to a strong constitutive promoter, and a terminator sequence; and (c) a sequence encoding isopentenyl transferase (ipt), wherein the modified TAL effector is designed to bind the desired nucleotide sequence within an intron of potato's ubiquitin-7 (Ubi7) gene.

17. The kit of claim 16, wherein the modified TAL effector comprises (a) a truncated C-terminal activation domain comprising a Fok1 endonuclease catalytic domain; (b) a codon-optimized target sequence binding domain comprising 16.5 repeat variable diresidues corresponding to the Ubi7 5′-untranslated intron sequence; and (c) an N-terminal region comprising a SV40 nuclear localization sequence.

18. The kit of claim 17, wherein the first binary vector further comprises a late blight resistance gene Vnt1 operably linked to its native promoter and terminator sequences.


SEQUENCE LISTING (37 sequences)

SEQ ID NO: 1 - DNA, 20 bp, Artificial Sequence
  Description of Artificial Sequence Synthetic primer
tcctaattttccccaccaca

SEQ ID NO: 2 - DNA, 20 bp, Artificial Sequence
  Description of Artificial Sequence Synthetic primer
aacagccggagaaactcaaa

SEQ ID NO: 3 - DNA, 568 bp, Solanum tuberosum
gttagaaatcttctctatttttggtttttgtctgtttagattctcgaattagctaatcaggtgctgttatagcccttaattttgagttttttttcggttgtcttgatggaaaaggcctaaaatttgagtttttttacgttggtttgatggaaaaggcctacaattggagttttccccgttgttttgatgaaaaagcccctagtttgagattttttttctgtcgattcgattctaaaggtttaaaattagagtttttacatttgtttgatgaaaaaggccttaaatttgagtttttccggttgatttgatgaaaaagccctagaatttgtgttttttcgtcggtttgattctgaaggcctaaaatttgagtttctccggctgttttgatgaaaaagccctaaatttgagtttctccggctgttttgatgaaaaagccctaaatttgagttttttccccgtgttttagattgtttggttttaattctcgaatcagctaatcagggagtgtgaaaagccctaaatttgagtttttttcgttgttctgattgttgtttttatgaatttgcag

SEQ ID NO: 4 - DNA, 18 bp, Artificial Sequence
  Description of Artificial Sequence Synthetic oligonucleotide
ttttgtctgtttagattc

SEQ ID NO: 5 - DNA, 18 bp, Artificial Sequence
  Description of Artificial Sequence Synthetic oligonucleotide
ttaagggctataacagca

SEQ ID NO: 6 - DNA, 3396 bp, Artificial Sequence
  Description of Artificial Sequence Synthetic polynucleotide
atggctcccaaaaagaagagaaaggtagaaccaggatcacctggtggacaatcacttatggacccaatacgaagcagaacgccatcaccagctagggaacttctctctggaccacagcctgatggagttcagccaactgcagatcgaggtgtttctccgccagccggtggccctttagatggactcccagcaagaagaacaatgtcccgtaccagactcccaagtccccctgccccgtcgccagccttttcagctgactccttctctgatcttcttaggcaatttgacccttctcttttcaatacatcccttttcgattcacttcctcctttcggcgcacatcatactgaggcagccaccggcgaatgggacgaagtccaaagtggtttaagggcagctgatgctccaccaccgacgatgagagtcgctgttaccgccgcacgtcctcctagagccaagccagcccctagaagacgagctgcgcaaccctccgatgcaagccctgcagctcaagtagaccttcgaacactaggttactcccagcaacaacaagaaaaaataaagccaaaggttagatcaacagttgcacaacatcacgaagccctagtcggacacggatttacacatgctcatatcgtggctctttcacaacatcctgcagctcttggaacagtcgctgtcaaatatcaggatatgattgctgcattgccagaagctactcacgaagctatcgtcggagttgggaaacaatggtcaggcgcaagagcattagaggcgcttctcaccgtagctggtgaattacgaggtcctccactccaattggatactgggcaattattaaaaatcgctaaacgaggtggagtcactgctgtcgaagccgttcatgcatggcgtaacgctctcacgggggccccactaaaccttaccccacaacaagttgtggcaatagcttctaatggtggtggtaaacaagcccttgagacggttcaaagacttctaccagttctttgtcaggcacatggattgaccccacaacaggtcgtagcaatcgcatctaacggaggtggtaagcaagctcttgaaacggtacaaagattacttcccgtgctttgtcaagctcatggactcactcctcaacaagtggtcgctattgcaagtaacggtggtggaaagcaagcactagaaaccgtccaacgactccttcctgttctctgtcaagcacatggtttgactcctcagcaggtcgtcgcaattgcatcaaacaatggaggcaaacaagctttagaaacagtacaaagactattgcccgttctttgccaagcgcatgggttaactcccgaacaagtcgttgccattgcaagtaacggaggaggtaaacaagctctcgaaacggttcaagcacttttacccgttctctgtcaagcacatggactcacacctgaacaagtagttgctatcgcatcgcatgatggtggaaaacaagcactggaaactgtacaaagacttttgccagttttatgtcaagcgcacggtcttactcctcaacaagttgtcgccattgcctctaatggaggtggaaaacaagctcttgaaactgtccagagacttctgcccgttctatgtcaggctcatgggctaacccctcaacaggttgttgcaatcgcatctaataatggaggaaaacaagctttagaaactgtccaacgactactgcccgttctctgccaagcacacggacttacaccacaacaggttgtagctatagctagcaatggtggcggtaaacaggctttggaaacagtacagcggcttctaccagtcttatgccaagcccacgggcttactcctcaacaagttgtcgccattgcctctaatggaggtggaaaacaagctcttgaaactgtccagagacttctgcccgttctatgtcaggctcatgggcttactcctgaacaggttgtcgcaatagcttcaaacggtggcggaaaacaagctcttgaaacagtgcaacgtctccttcccgtcctctgtcaggctcacggacttacgcccgaacaagttgttgctatagcttcgaatattggtggaaaacaagctctcgaaaccgtccaaaggctcctcccagtactttgccaagcacatggattaacccctgagcaagtagttgcaattgcctcgaacaatggaggaaagcaagcattagaaactgttcagagacttttgcctgtcctgtgtcaagcccacggtcttacaccagagcaggttgtcgctatagcttctaacattggtggaaagcaagctcttgagactgtgcaacgtttgcttccagtcctctgtcaagcacacggactcactccacaacaggtggttgcaattgcttcaaatggcggtggcaaacaagcattagagactgtacagagactacttcctgttctttgtcaagcacaagggctcacccctgagcaggtagtcgctatcgcctcaaatggtggcgggaagcaggccctggagactgttcagagactactgcccgtcctatgtcaggctcacggtctaacaccacaacaagtcgtcgcaatcgctagtcatgacggaggtcgacctgctctagagtcgatagtcgcacaactatcacgacctgatcccgctcttgcagcattgacaaacgatcatttagtcgcacttgcatgtttaggaggacgaccagcacttgatgccgttaagaaaggactaccgcacgcccctgcattgattaaaagaacaaacagacgaatcccggagagaacttcacatcgtgtagccaagcaacttgtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtgagacggaaatttaataacggcgagataaacttttga

SEQ ID NO: 7 - protein, 1131 aa, Artificial Sequence
  Description of Artificial Sequence Synthetic polypeptide
MAPKKKRKVEPGSPGGQSLMDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNGGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPQQVVAIASNGGGKQALETVQRLLPVLCQAQGLTPEQVVAIASNGGGKQALETVQRLLPVLCQAHGLTPQQVVAIASHDGGRPALESIVAQLSRPDPALAALTNDHLVALACLGGRPALDAVKKGLPHAPALIKRTNRRIPERTSHRVAKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 8 - DNA, 3396 bp, Artificial Sequence
  Description of Artificial Sequence Synthetic polynucleotide
atggctcccaaaaagaagagaaaggtagaaccaggatcacctggtggacaatcacttatggacccaatacgaagcagaacgccatcaccagctagggaacttctctctggaccacagcctgatggagttcagccaactgcagatcgaggtgtttctccgccagccggtggccctttagatggactcccagcaagaagaacaatgtcccgtaccagactcccaagtccccctgccccgtcgccagccttttcagctgactccttctctgatcttcttaggcaatttgacccttctcttttcaatacatcccttttcgattcacttcctcctttcggcgcacatcatactgaggcagccaccggcgaatgggacgaagtccaaagtggtttaagggcagctgatgctccaccaccgacgatgagagtcgctgttaccgccgcacgtcctcctagagccaagccagcccctagaagacgagctgcgcaaccctccgatgcaagccctgcagctcaagtagaccttcgaacactaggttactcccagcaacaacaagaaaaaataaagccaaaggttagatcaacagttgcacaacatcacgaagccctagtcggacacggatttacacatgctcatatcgtggctctttcacaacatcctgcagctcttggaacagtcgctgtcaaatatcaggatatgattgctgcattgccagaagctactcacgaagctatcgtcggagttgggaaacaatggtcaggcgcaagagcattagaggcgcttctcaccgtagctggtgaattacgaggtcctccactccaattggatactgggcaattattaaaaatcgctaaacgaggtggagtcactgctgtcgaagccgttcatgcatggcgtaacgctctcacgggggccccactaaaccttaccccacaacaagttgtggcaatagcttctaatggaggtggtaaacaagcccttgagacggttcaaagacttctaccagttctttgtcaggcacatggattgaccccacaacaggtcgtagcaatcgcatctaacattggtggtaagcaagctcttgaaacggtacaaagattacttcccgtgctttgtcaagctcatggactcactcctcaacaagtggtcgctattgcaagtaatattggtggaaagcaagcactagaaaccgtccaacgactccttcctgttctctgtcaagcacatggtttgactcctcagcaggtcgtcgcaattgcatcaaataacggaggcaaacaagctttagaaacagtacaaagactattgcccgttctttgccaagcgcatgggttaactcccgaacaagtcgttgccattgcaagtaacaatggaggtaaacaagctctcgaaacggttcaagcacttttacccgttctctgtcaagcacatggactcacacctgaacaagtagttgctatcgcatcgaataatggtggaaaacaagcactggaaactgtacaaagacttttgccagttttatgtcaagcgcacggtcttactcctcaacaagttgtcgccattgcctctcatgatggtggaaaacaagctcttgaaactgtccagagacttctgcccgttctatgtcaggctcatgggctaacccctcaacaggttgttgcaatcgcatctaatggtggaggaaaacaagctttagaaactgtccaacgactactgcccgttctctgccaagcacacggacttacaccacaacaggttgtagctatagctagcaatattggcggtaaacaggctttggaaacagtacagcggcttctaccagtcttatgccaagcccacgggcttactcctcaacaagttgtcgccattgcctctaacggaggtggaaaacaagctcttgaaactgtccagagacttctgcccgttctatgtcaggctcatgggcttactcctgaacaggttgtcgcaatagcttcaaacattggcggaaaacaagctcttgaaacagtgcaacgtctccttcccgtcctctgtcaggctcacggacttacgcccgaacaagttgttgctatagcttcgaatattggtggaaaacaagctctcgaaaccgtccaaaggctcctcccagtactttgccaagcacatggattaacccctgagcaagtagttgcaattgcctcgcacgatggaggaaagcaagcattagaaactgttcagagacttttgcctgtcctgtgtcaagcccacggtcttacaccagagcaggttgtcgctatagcttctaatatcggtggaaagcaagctcttgagactgtgcaacgtttgcttccagtcctctgtcaagcacacggactcactccacaacaggtggttgcaattgcttcaaataatggtggcaaacaagcattagagactgtacagagactacttcctgttctttgtcaagcacaagggctcacccctgagcaggtagtcgctatcgcctcacacgacggcgggaagcaggccctggagactgttcagagactactgcccgtcctatgtcaggctcacggtctaacaccacaacaagtcgtcgcaatcgctagtaatattggaggtcgacctgctctagagtcgatagtcgcacaactatcacgacctgatcccgctcttgcagcattgacaaacgatcatttagtcgcacttgcatgtttaggaggacgaccagcacttgatgccgttaagaaaggactaccgcacgcccctgcattgattaaaagaacaaacagacgaatcccggagagaacttcacatcgtgtagccaagcaacttgtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtgagacggaaatttaataacggcgagataaacttttga

SEQ ID NO: 9 - protein, 1131 aa, Artificial Sequence
  Description of Artificial Sequence Synthetic polypeptide
MAPKKKRKVEPGSPGGQSLMDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNIGGKQALETVQRLLPVLCQAHGLTPQQVVAIASNIGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNNGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNIGGKQALETVQRLLPVLCQAHGLTPQQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPQQVVAIASNNGGKQALETVQRLLPVLCQAQGLTPEQVVAIASHDGGKQALETVQRLLPVLCQAHGLTPQQVVAIASNIGGRPALESIVAQLSRPDPALAALTNDHLVALACLGGRPALDAVKKGLPHAPALIKRTNRRIPERTSHRVAKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 10 - DNA, 1980 bp, Solanum tuberosum
atggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccgtttcgcccctgacgaacccagaaagggttgtgatgttcttgtggaggcacttgaaagggagggggttacggatgtatttgcgtacccaggaggtgcttctatggagattcatcaggctttgacacgttcgaatattattcgtaatgtgctgccacgtcatgagcaaggtggtgtgtttgctgcagagggttacgcacgggcgactgggttccctggtgtttgcattgctacctctggtccgggagctacgaatcttgttagtggtcttgcggatgctttgttggatagtattccgattgttgctattacgggtcaagtgccgaggaggatgattggtactgatgcgtttcaggaaacgcctattgttgaggtaacgagatctattacgaagcataattatcttgttatggatgtagaggatattcctagggttgttcgtgaagcgttttttctagcgaaatcgggacggcctgggccggttttgattgatgtacctaaggatattcagcaacaattggtgatacctaattgggatcagccaatgaggttgcctggttacatgtctaggttacctaaattgcctaatgagatgcttttggaacaaattattaggctgatttcggagtcgaagaagcctgttttgtatgtgggtggtgggtgtttgcaatcaagtgaggagctgagacgatttgtggagcttacgggtattcctgtggcgagtactttgatgggtcttggagcttttccaactggggatgagctttcccttcaaatgttgggtatgcatgggactgtgtatgctaattatgctgtggatggtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggagacaggagttaacggaacagaaggtgaagtacccattgagttttaagacttttggtgaagccatccctccacaatatgctattcaggttcttgatgagttaactaacggaaatgccattattagtactggtgtggggcaacaccagatgtgggctgcccaatactataagtacaaaaagccacaccaatggttgacatctggtggattaggagcaatgggatttggtttgcctgctgcaataggtgcggctgttggaagaccgggtgagattgtggttgacattgatggtgacgggagttttatcatgaatgtgcaggagttagcaacaattaaggtggagaatctcccagttaagattatgttgctgaataatcaacacttgggaatggtggttcaatgggaggatcgattctataaggctaacagagcacacacttacttgggtgatcctgctaatgaggaagagatcttccctaatatgttgaaattcgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccagtggcggtgctttcaaagatgtgatcacagagggtgatgggagacgttcatattga

SEQ ID NO: 11 - protein, 659 aa, Solanum tuberosum
MAAAASPSPCFSKTLPPSSSKSSTILPRSTFPFHNHPQKASPLHLTHTHHHRRGFAVSNVVISTTTHNDVSEPETFVSRFAPDEPRKGCDVLVEALEREGVTDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYMSRLPKLPNEMLLEQIIRLISESKKPVLYVGGGCLQSSEELRRFVELTGIPVASTLMGLGAFPTGDELSLQMLGMHGTVYANYAVDGSDLLLAFGVRFDDRVTGKLEAFASRAKIVHIDIDSAEIGKNKQPHVSICADIKLALQGLNSILEGKEGKLKLDFSAWRQELTEQKVKYPLSFKTFGEAIPPQYAIQVLDELTNGNAIISTGVGQHQMWAAQYYKYKKPHQWLTSGGLGAMGFGLPAAIGAAVGRPGEIVVDIDGDGSFIMNVQELATIKVENLPVKIMLLNNQHLGMVVQWEDRFYKANRAHTYLGDPANEEEIFPNMLKFAEACGVPAARVSHRDDLRAAIQKMLDTPGPYLLDVIVPHQEHVLPMIPSGGAFKDVITEGDGRRSY

SEQ ID NO: 12 - DNA, 1980 bp, Solanum tuberosum
atggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccgtttcgcccctgacgaacccagaaagggttgtgatgttcttgtggaggcacttgaaagggagggggttacggatgtatttgcgtacccaggaggtgcttctatggagattcatcaggctttgacacgttcgaatattattcgtaatgtgctgccacgtcatgagcaaggtggtgtgtttgctgcagagggttacgcacgggcgactgggttccctggtgtttgcattgctacctctggtccgggagctacgaatcttgttagtggtcttgcggatgctttgttggatagtattccgattgttgctattacgggtcaagtgccgaggaggatgattggtactgatgcgtttcaggaaacgcctattgttgaggtaacgagatctattacgaagcataattatcttgttatggatgtagaggatattcctagggttgttcgtgaagcgttttttctagcgaaatcgggacggcctgggccggttttgattgatgtacctaaggatattcagcaacaattggtgatacctaattgggatcagccaatgaggttgcctggttacatgtctagattacctaaattgcctaatgagatgcttttggaacaaattattaggctgatttcggagtcgaagaagcctgttttgtatgtgggtggtgggtgtttgcaatcaagtgaggagctgagacgatttgtggagcttacgggtattcctgtggcgagtactttgatgggtcttggagcttttccaactggggatgagctttcccttcaaatgttgggtatgcatgggactgtgtatgctaattatgctgtggatggtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggagacaggagttaacggaacagaaggtgaagtacccattgagttttaagacttttggtgaagccatccctccacaatatgctattcaggttcttgatgagttaactaacggaaatgccattattagtactggtgtggggcaacaccagatgtgggctgcccaatactataagtacaaaaagccacaccaatggttgacatctggtggattaggagcaatgggatttggtttgcctgctgcaataggtgcggctgttggaagaccgggtgagattgtggttgacattgatggtgacgggagttttatcatgaatgtgcaggagttagcaacaattaaggtggagaatctcccagttaagattatgttgctgaataatcaacacttgggaatggtggttcaactggaggatcgattctataaggctaacagagcacacacttacttgggtgatcctgctaatgaggaagagatcttccctaatatgttgaaattcgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccattggcggtgctttcaaagatgtgatcacagagggtgatgggagacgttcatattga

SEQ ID NO: 13 - protein, 659 aa, Solanum tuberosum
MAAAASPSPCFSKTLPPSSSKSSTILPRSTFPFHNHPQKASPLHLTHTHHHRRGFAVSNVVISTTTHNDVSEPETFVSRFAPDEPRKGCDVLVEALEREGVTDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYMSRLPKLPNEMLLEQIIRLISESKKPVLYVGGGCLQSSEELRRFVELTGIPVASTLMGLGAFPTGDELSLQMLGMHGTVYANYAVDGSDLLLAFGVRFDDRVTGKLEAFASRAKIVHIDIDSAEIGKNKQPHVSICADIKLALQGLNSILEGKEGKLKLDFSAWRQELTEQKVKYPLSFKTFGEAIPPQYAIQVLDELTNGNAIISTGVGQHQMWAAQYYKYKKPHQWLTSGGLGAMGFGLPAAIGAAVGRPGEIVVDIDGDGSFIMNVQELATIKVENLPVKIMLLNNQHLGMVVQLEDRFYKANRAHTYLGDPANEEEIFPNMLKFAEACGVPAARVSHRDDLRAAIQKMLDTPGPYLLDVIVPHQEHVLPMIPIGGAFKDVITEGDGRRSY

SEQ ID NO: 14 - DNA, 13057 bp, Artificial Sequence
  Description of Artificial Sequence Synthetic polynucleotide
tggcaggatatataccggtgtaaacgaagtgtgtgtggttgatccaaaatctatcgtacctttagaaagtgtagctatgaaggatagtctcacttatgaagaactacctattgagattcttgatcgtcaggtccgaaggttgagaaaaatagaagtcgcttcagttacggctttgtggaggagtaagggtaccagttatacaccctacattctactcgagtcattatgatgatgtctcacgaccaaatcaaatcaaagttaaataaatatcgaaccgaacgcccactctgtatgagtatggcaaaagattttgagagaatcaagttgcataaaagcctaattttcatggaacatacaaattgagtctcataatagcccaaactcacagccatgaacccaaattgggtaaagttttgcaagacgttcatcaaacagttaggaaacataaaatggcgctagatatataataaatttttttaacatatggtgtgattgatagttatatactaaagatgtttgcttagttacgtaattttttcaaaaaaaaaaggtacattatcaatcatcagtcacaaaatattaaaagttactgtttgttttttaaattccatgtcgaatttaattgaatgacacttaaattgggacgaacggtgtaatttcttttgactattctactagtatctatccacagcacgtgttgttcctttcttctttcgtttttcatttacttgacattattaggagacttggccctgaactccaactattctaagctgacctttcttttcctttaccaattatcttcttctttctaatttcgttttacgcgtagtactgcctgaattttctgactttcaacgtttgttattcatgcttgaaaacgaaataccagctaacaaaagatgaattattgtgtttacaagacttgggccgttgactcttactttcccttcctcatcctcacatttagaaaaaagaaatttaacgaaaaattaaaggagatggctgaaattcttctcacagcagtcatcaataaatcaatagaaatagctggaaatgtactctttcaagaaggtacgcgtttatattggttgaaagaggacatcgattggctccagagagaaatgagacacattcgatcatatgtagacaatgcaaaggcaaaggaagttggaggcgattcaagggtgaaaaacttattaaaagatattcaacaactggcaggtgatgtggaggatctattagatgagtttcttccaaaaattcaacaatccaataagttcatttgttgccttaagacggtttcttttgccgatgagtttgctatggagattgagaagataaaaagaagagttgctgatattgaccgtgtaaggacaacttacagcatcacagatacaagtaacaataatgatgattgcattccattggaccggagaagattgttccttcatgctgatgaaacagaggtcatcggtctggaagatgacttcaatacactacaagccaaattacttgatcatgatttgccttatggagttgtttcaatagttggcatgcccggtttgggaaaaacaactcttgccaagaaactttataggcatgtctgtcatcaatttgagtgttcgggactggtctatgtttcacaacagccaagggcgggagaaatcttacatgacatagccaaacaagttggactgacggaagaggaaaggaaagaaaacttggagaacaacctacgatcactcttgaaaataaaaaggtatgttattctcttagatgacatttgggatgttgaaatttgggatgatctaaaacttgtccttcctgaatgtgattcaaaaattggcagtaggataattataacctctcgaaatagtaatgtaggcagatacataggaggggatttctcaatccacgtgttgcaacccctagattcagagaaaagctttgaactctttaccaagaaaatctttaattttgttaatgataattgggccaatgcttcaccagacttggtaaatattggtagatgtatagttgagagatgtggaggtataccgctagcaattgtggtgactgcaggcatgttaagggcaagaggaagaacagaacatgcatggaacagagtacttgagagtatggctcataaaattcaagatggatgtggtaaggtattggctctgagttacaatgatttgcccattgcattaaggccatgtttcttgtactttggtctttaccccgaggaccatgaaattcgtgcttttgatttgacaaatatgtggattgctgagaagctgatagttgtaaatactggcaatgggcgagaggctgaaagtttggcggatgatgtcctaaatgatttggtttcaagaaacttgattcaagttgccaaaaggacatatgatggaagaatttcaagttgtcgcatacatgacttgttacatagtttgtgtgtggacttggctaaggaaagtaacttctttcacacggagcacaatgcatttggtgatcctagcaatgttgctagggtgcgaaggattacattctactctgatgataatgccatgaatgagttcttccatttaaatcctaagcctatgaagcttcgttcacttttctgtttcacaaaagaccgttgcatattttctcaaatggctcatcttaacttcaaattattgcaagtgttggttgtagtcatgtctcaaaagggttatcagcatgttactttccccaaaaaaattgggaacatgagttgcctacgttatgtgcgattggagggggcaattagagtaaaattgccaaatagtattgtcaagctcaaatgtctagagaccctggatatatttcatagctctagtaaacttccttttggtgtttgggagtctaaaatattgagacatctttgttacacagaagaatgttactgtgtctcttttgcaagtccattttgccgaatcatgcctcctaataatctacaaactttgatgtgggtggatgataaattttgtgaaccaagattgttgcaccgattgataaatttaagaacattgtgtataatggatgtatccggttctaccattaagatattatcagcattgagccctgtgcctagagcgttggaggttctgaagctcagatttttcaagaacacgagtgagcaaataaacttgtcgtcccatccaaatattgtcgagttgggtttggttggtttctcagcaatgctcttgaacattgaagcattccctccaaatcttgtcaagcttaatcttgtcggcttgatggtagacggtcatctattggcagtgcttaagaaattgcccaaattaaggatacttatattgctttggtgcagacatgatgcagaaaaaatggatctctctggtgatagctttccgcaacttgaagttttgtatattgaggatgcacaagggttgtctgaagtaacgtgcatggatgatatgagtatgcctaaattgaaaaagctatttcttgtacaaggcccaaacatttccccaattagtctcagggtctcggaacggcttgcaaagttgagaatatcacaggtactataaataattatttacgtttaatatccatgatttttttaaatttgtatttagttcatcaactaaatattccatgtctaataaattgcagggatgcctttgaaaatgattctgtgttggagagaatcttctgatgcctgttggtattataatactaataataagagaaaaagtttgattactgtttcaagttaattgcttgtgatttgtaaaaacaaattacttttatatttctctttgttttattttatgtttatttatctttaattaatggagtaataaaataaaaatcttattttcaatagaaaaaagtagaccttatttgtggtgcatgtatggtatctttttgaaatttttgatatatttgctctttgattcgaatttcttgcttatatgatgatttgcataaatataaaatattatacaaatacctatgggttggaaaatatagaaatatgccaatcaaatgtatacaaaaatcattaatagatagaatcgtaaaagatatacaaatgagaaatgcttgactaagaagcttcgtgcaacctctcacactgagcacaatgcatttggtgatctcggcactattgctgttacttgtaagactacgttccccaataagtctttccaaacggcttgcaaagctgagaatatgaaaatctcataggttagtttgctgcgttaattatttacatttaatatgctcgataaggtgattttaaaaaaatttgtactagttaattcatgaactaaatatttcatttaatactccataattctgaatatggaaaataaataatatttaataacaagaataaaatgataaattattcattgattttataaattggataaatattattaaatattcttaaataatataatgaacaagtgaagatgaacggagggagtatgaagcctcttttcaaaggggccccaagtgtctgagacaaccaaaactgaaagtgggaaaccaaactctaagtcaaagactttatatacaaaatggtataaatataattatttaatttactatcgggttatcgattaacccgttaagaaaaaacttcaaaccgttaagaaccgataacccgataacaaaaaaaatctaaatcgttatcaaaaccgctaaactaataacccaatattgataaaccaataactttttttattcgggttatcggtttcagttctgtttggaacaatcctagtgtcctaattattgttttgagaaccaagaaaacaaaaacttacgtcgcaaatatttcagtaaatacttgtatatctcagtgataattgatttccaacatgtataattatcatttacgtaataatagatggtttccgaaacttacgcttcccttttttcttttgcagtcgtatggaataaaagttggatatggaggcattcccgggccttcaggtggaagagacggagctgcttcacaaggagggggttgttgtacttgaaaatgggcatttattgttcgcaaacctatcatgttcctatggttgtttatttgtagtttggtgttcttaatatcgagtgttctttagtttgttccttttaatgaaaggataatatctgtgcaaaaataagtaaattcggtacataaagacatttttttttgcattttctgtttatggagttgtcaaatgtgaatttatttcatagcatgtgagtttcctctcctttttcatgtgcccttgggccttgcatgtttcttgcaccgcagtgtgccagggctgtcggcagatggacataaatggcacaccgctcggctcgtggaaagagtatggtcagtttcattgataagtatttactcgtattcggtgtttacatcaagttaatatgttcaaacacatgtgatatcatacatccattagttaagtataaatgccaactttttacttgaatcgccgaataaatttacttacgtccaatatttagttttgtgtgtcaaacatatcatgcactatttgattaagaataaataaacgatgtgtaatttgaaaaccaattagaaaagaagtatgacgggattgatgttctgtgaaatcactggtaaattggacggacgatgaaatttgatcgtccatttaagcatagcaacatgggtctttagtcatcatcattatgttataattattttcttgaaacttgatacaccaactttcattgggaaagtgacagcatagtataaactataatatcaattctggcaatttcgaattattccaaatctcttttgtcatttcatttcctcccctatgtctgcaagtaccaattatttaagtacaaaaaatcttgattaaacaatttattttctcactaataatcacatttaatcatcaacggttcatacacgtctgtcactctttttttattctctcaagcgcatgtgatcataccaattatttaaatacaaaaaatcttgattaaacaattcagtttctcactaataatcacatttaatcatcaacggttcatacacatccgtcactctttttttattctctcaagcgcatgtgatcataccaattatttaaatacaaaaaatcttgattaaacaattcattttctcactaataatcacatttaatcatcaacggtttatacacgtccgccactctttttttattctctcaagcgtatgtgatcatatctaactctcgtgcaaacaagtgaaatgacgttcactaataaataatcttttgaatactttgttcagtttaatttatttaatttgataagaatttttttattattgaatttttattgttttaaattaaaaataagttaaatatatcaaaatatcttttaattttatttttgaaaaataacgtagttcaaacaaattaaaattgagtaactgtttttcgaaaaataatgattctaatagtatattctttttcatcattagatattttttttaagctaagtacaaaagtcatatttcaatccccaaaatagcctcaatcacaagaaatgcttaaatccccaaaataccctcaatcacaagacgtgtgtaccaatcatacctatggtcctctcgtaaattccgacaaaatcaggtctataaagttacccttgatatcagtattataaaactaaaaatctcagctgtaattcaagtgcaatcacactctaccacacactctctagtagagagatcagttgataacaagcttgttaacggatccataattgtaactgatttattcttgaataacaacttcaatgaaatcaagcaacaaagctgatttcaacataaaaaaacagaacaagaaaacaaaaacagagcatcatccatcaaagtgtaatctcagcagattcaatagagactacaagattttgcacttgtacataatcatcagtgtcaccggtataaagcatcatgatctgaccatcgggtaggatggtagcggacccagtccagacaccgttaatatcgtaccattgatcaggaaccatggcaaaaggcaagtagagccagtggatcaagtccttggatacggcatggccccatgtgatatttccccaaatagctgaatctggattgtattgataaaaaagatgataccatcccttgtggtacaatggaccattaggatcgttcatccaatttttttgaggttgaaaatggtaagcagttctttgccagctaagcatagcattggaccacgcataagaaacgtgactagcattgacgacatctcgaaaagtcttatcggagactccctgagaaacacctcttgacggcggcgccggcgaacgggagttactctgcaagtccggtgactggttgttgaggatcggaaagaaggctacagaaagcaaaaggaaagaggagaggaaaatgccggagatgattttaagggacttccggtggccggaatcgggttgatccgggaggaatgtgtaatgggaggcggagttttccgggtcataactggaatggtactgcgtggccatactcgtgcctaaaatggcgaataagtagagtataacactacatattctccctctcttccctttcttgatgggacatcggtgaaataaccttcaaatgaaaaaaagaatgaagaagatatggcttgatgaagaactctttatccagaaatggtactctagcttctaagccccacgcggatgtagccttgtttgctcttaaacagtcatactggtgaagcgcttttatcttgcgacatgtttccgtgtggaactcttccttgtttggagccttgtggaagtacaagtagccaccaaaaatttcgtcagcaccttcccctgatatgaccatcttcactcctagtgatttaatcttacgtgacataaggaacataggagtgctggctcttattgttgttacatcatacgtctcgatatgatatataacatcttcaatagcatcaatcccgtcctgaacagtaaagtgaaactcgtggtgaacggttcctaaaaagtcagcaacttcttttgcagccttgagatctggtgagccctcgagaacattttgaagttttccctccggggcacttgtactctagcaagaacggagggcttaggagatggtacaatcccgcttggttctctgaagcaattccttccactccttatgacactttggttctgaggcgtgccttcgaaaatgctgttatcaaacggttgatgactgatgtcccctttggcgttctgctctcggggggacttgattcgtctttggttgcttctgtcactactcgatacttggctggaacaaaagctgctaagcaatggggagcacaacttcattccttctgtgttggtctcgagggctcaccagatctcaaggctgcaaaagaagttgctgactttttaggaaccgttcaccacgagtttcactttactgttcaggacgggattgatgctattgaagatgttatatatcatatcgagacgtatgatgtaacaacaataagagccagcactcctatgttccttatgtcacgtaagattaaatcactaggagtgaagatggtcatatcaggggaaggtgctgacgaaatttttggtggctacttgtacttccacaaggctccaaacaaggaagagttccacacggaaacatgtcgcaagataaaagcgcttcaccagtatgactgtttaagagcaaacaaggctacatccgcgtggggcttagaagctagagtaccatttctggataaagagttcttcatcaagccatatcttcttcattctttttttcatttgaaggttatttcaccgatgtcccatcaagaaagggaagagagggagaatatgtagtgttatactctacttattcgccattttaggcacgagtatggccacgcagtaccattccagttatgacccggaaaactccgcctcccattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatattaacggtgtctggactgggtccgctaccatcctacccgatggtcagatcatgatgctttataccggtgacactgatgattatgtacaagtgcaaaatcttgtagtctctattgaatctgctgagattacactttgatggatgatgctctgtttttgttttcttgttctgtttttttatgttgaaatcagctttgttgcttgatttcattgaagttgttattcaagaataaatcagttacaattatactagtccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacgagctccctatttttttactatattatactcaacccaatgagcataaagactgtaaaatctcaaattcctgagaagcatatttatcgatcccacagacttgatagttccataatccatacgctgcagccaaattgctagtgtgttgaacatttaacacgtagagaactagaaaagatataaaactaagattgatatccaaaatagacgagaacaataagcaaaaactcttagttttgaaataaatcaacaatcccgagggttgtcacatacatcaaaaacgaaaatccatatagcaaaaaaaactctaaattaccgttcgacaaaaagagaaaactgataggacatttgctaaacattaaaatcaatatgaacgtctcccatcaccctctgtgatcacatctttgaaagcaccgccaatgggaatcataggtagaacgtgctcctgatgaggtacaatcacatccaacaagtatggcccaggagtgtctaacatcttttgaatggcagctctaagatcatccctgtgtgacactcttgcagcaggtacgccacaagcctctgcgaatttcaacatattagggaagatctcttcctcattagcaggatcacccaagtaagtgtgtgctctgttagccttatagaatcgatcctccagttgaaccaccattcccaagtgttgattattcagcaacataatcttaactgggagattctccaccttaattgttgctaactcctgcacattcatgataaaactcccgtcaccatcaatgtcaaccacaatctcacccggtcttccaacagccgcacctattgcagcaggcaaaccaaatcccattgctcctaatccaccagatgtcaaccattggtgtggctttttgtacttatagtattgggcagcccacatctggtgttgccccacaccagtactaataatggcatttccgttagttaactcatcaagaacctgaatagcatattgtggagggatggcttcaccaaaagtcttaaaactcaatgggtacttcaccttctgttccgttaactcctgtctccaagcagaaaagtccaacttcagcttaccttctttaccctccaatatggaattcaaaccctgtaatgccaacttgatatctgcacaaatggaaacatgaggttgcttgttctttccaatctcagccgaatcaatatcaatgtggacaattttcgctcggctagcaaaagcttccaatttaccagtaactcgatcatcaaacctcaccccaaatgcaagcaacaaatcactaccatccacagcataattagcatacacagtcccatgcatacccaacatttgaagggaaagctcatccccagttggaaaagctccaagacccatcaaagtactcgccacaggaatacccgtaagctccacaaatcgtctcagctcctcacttgattgcaaacacccaccacccacatacaaaacaggcttcttcgactccgaaatcagcctaataatttgttccaaaagcatctcattaggcaatttaggtaatctagacatgtaaccaggcaacctcattggctgatcccaattaggtatcaccaattgttgctgaatatccttaggtacatcaatcaaaaccggcccaggccgtcccgatttcgctagaaaaaacgcttcacgaacaaccctaggaatatcctctacatccataacaagataattatgcttcgtaatagatctcgttacctcaacaataggcgtttcctgaaacgcatcagtaccaatcatcctcctcggcacttgacccgtaatagcaacaatcggaatactatccaacaaagcatccgcaagaccactaacaagattcgtagctcccggaccagaggtagcaatgcaaacaccagggaacccagtcgcccgtgcgtaaccctctgcagcaaacacaccaccttgctcatgacgtggcagcacattacgaataatattcgaacgtgtcaaagcctgatgaatctccatagaagcacctcctgggtacgcaaatacatccgtaaccccctccctttcaagtgcctccacaagaacatcacaaccctttctgggttcgtcaggggcgaaacgggaaacgaatgtttcaggttcagaaacgtcgttatgggtggtagtggatatgacgacattggaaacggcgaaaccacgacgatgatgatgggtgtgggtgagatgaaggggtgaggctttttgagggtgattgtggaaagggaaggtagatctaggaagaatggtggaagatttggaggaagatggaggtagggttttggagaaacatggagatggtgaggcagcagccgccatacctccacgtagacggagcaccaaatggagggtagactccttctggatgttgtaatcagctagagtacgtccgtcctccaactgctttccggcgaagataagcctttgctgatccgggggaattccttccttatcctggatcttagccttaacgttgtcgattgtatcagaactttccacctctagggtgatagtctttccggtgagagttttcacaaagatctgcatctgcaaattcataaaaacaacaatcagaacaacgaaaaaaactcaaatttagggcttttcacactccctgattagctgattcgagaattaaaaccaaacaatctaaaacacggggaaaaaactcaaatttagggctttttcatcaaaacagccggagaaactcaaatttagggctttttcatcaaaacagccggagaaactcaaattttaggccttcagaatcaaaccgacgaaaaaacacaaattctagggctttttcatcaaatcaaccggaaaaactcaaatttaaggcctttttcatcaaacaaatgtaaaaactctaattttaaacctttagaatcgaatcgacagaaaaaaaatctcaaactaggggctttttcatcaaaacaacggggaaaactccaattgtaggccttttccatcaaaccaacgtaaaaaaactcaaattttaggccttttccatcaagacaaccgaaaaaaaactcaaaattaagggctataacagcacctgattagctaattcgagaattgacaggatatatggtactgtaaac

SEQ ID NO: 15 - DNA, 3014 bp, Artificial Sequence
  Description of Artificial Sequence Synthetic polynucleotide
atttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaaccaatactagtatgttaagggctataacagcacctgattagctaattcgagaatctaaacagacaaaaaccaaagtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaaagcttttgattttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaatagg

SEQ ID NO: 16 - DNA, 20 bp, Artificial Sequence
  Description of Artificial Sequence Synthetic primer
aaggcattcattcccatttg

SEQ ID NO: 17 - DNA, 20 bp, Artificial Sequence
  Description of Artificial Sequence Synthetic primer
gacccacactttgccgtaat

SEQ ID NO: 18 - protein, 34 aa, Unknown
  Description of Unknown Central repeat domain polypeptide
LTPEQVVAIASHDGGKQALETVQRLLPVLCQAHG

SEQ ID NO: 19 - DNA, 1018 bp, Artificial Sequence
  Description of Artificial Sequence Synthetic polynucleotide
gtttacagtaccatatatcctgtcaattctcgaattagctaatcaggtgctgttatagcccttaattttgagttttttttcggttgtcttgatggaaaaggcctaaaatttgagtttttttacgttggtttgatggaaaaggcctacaattggagttttccccgttgttttgatgaaaaagcccctagtttgagattttttttctgtcgattcgattctaaaggtttaaaattagagtttttacatttgtttgatgaaaaaggccttaaatttgagtttttccggttgatttgatgaaaaagccctagaatttgtgttttttcgtcggtttgattctgaaggcctaaaatttgagtttctccggctgttttgatgaaaaagccctaaatttgagtttctccggctgttttgatgaaaaagccctaaatttgagttttttccccgtgttttagattgtttggttttaattctcgaatcagctaatcagggagtgtgaaaagccctaaatttgagtttttttcgttgttctgattgttgtttttatgaatttgcagatgcagatctttgtgaaaactctcaccggaaagactatcaccctagaggtggaaagttctgatacaatcgacaacgttaaggctaagatccaggataaggaaggaattcccccggatcagcaaaggcttatcttcgccggaaagcagttggaggacggacgtactctagctgattacaacatccagaaggagtctaccctccatttggtgctccgtctacgtggaggtatggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccg

SEQ ID NO: 20 - DNA, 66 bp, Artificial Sequence
  Description of Artificial Sequence Synthetic oligonucleotide
atgttaagggctataacagcacctgattagctaattcgagaatctaaacagacaaaaaccaaagtc

SEQ ID NO: 21 - DNA, 60 bp, Artificial Sequence
  Description of Artificial Sequence Synthetic oligonucleotide
tttggtttttgtctgtttagattctcgaattagctaatcaggtgctgttatagcccttaa

SEQ ID NO: 22 - DNA, 60 bp, Artificial Sequence; identical to SEQ ID NO: 21
  Description of Artificial Sequence Synthetic oligonucleotide

SEQ ID NO: 23 - DNA, 60 bp, Artificial Sequence
  Description of Artificial Sequence Synthetic oligonucleotide
tttggtttttgtctgtttagattctcgaattagttaatcaggtgctgttatagcccttaa

SEQ ID NO: 24 - DNA, 55 bp, Artificial Sequence
  Description of Artificial Sequence Synthetic oligonucleotide
tttggtttttgtctgtttagattctcgaataatcaggtgctgttatagcccttaa

SEQ ID NO: 25 - DNA, 53 bp, Artificial Sequence
  Description of Artificial Sequence Synthetic oligonucleotide
tttggtttttgtctgtttagattctcgaaatcaggtgctgttatagcccttaa

SEQ ID NO: 26 - DNA, 45 bp, Artificial Sequence
  Description of Artificial Sequence Synthetic oligonucleotide
tttggtttttgtctgtttagattctcgaactgttatagcccttaa

SEQ ID NO: 27 - DNA, 52 bp, Artificial Sequence
  Description of Artificial Sequence Synthetic oligonucleotide
tttggtttttgtctgtttagattctcgaatcaggtgctgttatagcccttaa

SEQ ID NO: 28 - DNA, 56 bp, Artificial Sequence
  Description of Artificial Sequence Synthetic oligonucleotide
tttggtttttgtctgtttagattctcgaattaatcaggtgctgttatagcccttaa

SEQ ID NO: 29 - DNA, 52 bp, Artificial Sequence
  Description of Artificial Sequence Synthetic oligonucleotide
tttggtttttgtctgcttagattctcgaattaggtgctgttatagcccttaa

SEQ ID NO: 30 - DNA, 53 bp, Artificial Sequence
  Description of Artificial Sequence Synthetic oligonucleotide
tttggtttttgtctgtttagattctcgaattcaggtgctgttatagcccttaa

SEQ ID NO: 31 - DNA, 55 bp, Artificial Sequence
  Description of Artificial Sequence Synthetic oligonucleotide
tttggtttttgtctgtttagattctcgaattatcaggtgctgttatagcccttaa

SEQ ID NO: 32 - DNA, 56 bp, Artificial Sequence; identical to SEQ ID NO: 28
  Description of Artificial Sequence Synthetic oligonucleotide

SEQ ID NO: 33 - DNA, 58 bp, Artificial Sequence
  Description of Artificial Sequence Synthetic oligonucleotide
tttggtttttgtctgtttagattctcgaattataatcaggtgctgttatagcccttaa

SEQ ID NO: 34 - DNA, 61 bp, Artificial Sequence
  Description of Artificial Sequence Synthetic oligonucleotide
tttggtttttgtctgtttagattctcgaattagcctaatcaggtgctgttatagcccttaa

SEQ ID NO: 35 - DNA, 1450 bp, Artificial Sequence
  Description of Artificial Sequence Synthetic polynucleotide
ctgatttctattataatttctattaattgccttcaaatttctctttcaaggttagaaatcttctctattttttggtttttgtctgtttagattctcgaattagctaatcaggtgctgttaaagccctaaaatttgagttttttttccgtcgaattgatgctaaaggcttaaaattagagttttttcgtcggtttgactctgaaggcctaaaatttggggttttccgggtgatttgatgataaagccctagaatttgagtttttttatttgtcggtttgatgaaaaaggccttaaatttaatttttttcccggttgatttgatgaaaaagccctagaatttgtgttttttcgtcggtttgattctaaaggcctaaaatttgagtttttccggttgttttgatgaaaaagccctaaaatttgagttttttccccgtgttttagattgtttggttttaattcttgaatcagataatcagggagtgtgaaaagccctaaaatttgagtttttttcgttgttctgattgttgtttttatgaatttgattctcgaattagctaatcaggtgctgttatagcccttaattttgagttttttttcggttgtcttgatggaaaaggcctaaaatttgagtttttttacgttggtttgatggaaaaggcctacaattggagttttccccgttgttttgatgaaaaagcccctagtttgagattttttttctgtcgattcgattctaaaggtttaaaattagagtttttacatttgtttgatgaaaaaggccttaaatttgagtttttccggttgatttgatgaaaaagccctagaatttgtgtttttcgtcggtttgattctgaaggtttgattctgaaggcctaaaatttgagtttctccggctgttttgatgaaaaagccctaaatttgagtttctccggctgttttgatgaaaaagccctaaatttgagttttttccccgtgttttagattgtttggttttaattctcgaatcagctaatcagggagtgtgaaaagccctaaatttgagtttttttcgttgttctgattgttgtttttatgaatttgcagatgcagatctttgtgaaaactctcaccggaaagactatcaccctagaggtggaaagttctgatacaatcgacaacgttaaggctaagatccaggataaggaaggaattcccccggatcagcaaaggcttatcttcgccggaaagcagttggaggacggacgtactctagctgattacaacatccagaaggagtctaccctccatttggtgctccgtctacgtggaggtatggcggctgctgcctcaccatctccatgtttctccaaaaccctacctccatcttcctccaaatcttccaccattcttcctagatctaccttccctttccacaatcaccctcaaaaagcctcaccccttcatctcaccc

SEQ ID NO: 36 - DNA, 891 bp, Artificial Sequence
  Description of Artificial Sequence Synthetic polynucleotide
ttttcatcttctatctgatttctattataatttctattaattgccttcaaatttctctttcaaggttagaaatcttctctattttttggtttttgtctgtttagattctcgaattagctaatcaggtgctgttaaagccctaaaatttgagttttttttccgccgaattgatgctaaaggcttaaaattagggttttttcgtcggtttgactctgaaggcctaaaatttggggttttccgggtgatttgatgataaagccctagaatttgagtttttttatttgtcggtttgatgaaaaaggccttaaatttaatttttttcccggttgatttgatgaaaaagccctagaatttgtgttttttcgtcggtttgattctaaaggcctaaaatttgagtttttccggttgttttgatgaaaaagccctaaaatttgagttttttccccgtgttttagattgtttggttttaattcttgaatcagataatcagggagtgtgaaaagccctaaatttgagtttttttcgttgttctgattgttgtttttatgaatttgcagatgcagatctttgtgaaaactctcaccggaaagactatcaccctagaggtggaaacaatcgacaacgttaaggctaagatccaggataaggaaggaattcccccggatcagcaaaggcttatcttcgccggaaagcagttggaggacggacgtactctagctgattacaacatccagaaggagtctaccctccatttggtgctccgtctacgtggaggtatggcggctgctgcctcaccatctccatgcttctccaaaaccctacctccatcttcctccaaatcttccaccattcttcctagatctaccttccctttccacaatcaccctcaaa

SEQ ID NO: 37 - DNA, 900 bp, Artificial Sequence
  Description of Artificial Sequence Synthetic polynucleotide
ttttcatcttctatctgatttctattataatttctattaattgccttcaaatttctctttcaaggttagaaatcttctctattttttggtttttgtctgtttagattctcgaattagctaatcaggtgctgttaaagccctaaaatttgagttttttttccgccgaattgatgctaaaggcttaaaattagggttttttcgtcggtttgactctgaaggcctaaaatttggggttttccgggtgatttgatgataaagccctagaatttgagtttttttatttgtcggtttgatgaaaaaggccttaaatttaatttttttcccggttgatttgatgaaaaagccctagaatttgtgttttttcgtcggtttgattctaaaggcctaaaatttgagtttttccggttgttttgatgaaaaagccctaaaatttgagttttttccccgtgttttagattgtttggttttaattcttgaatcagataatcagggagtgtgaaaagccctaaatttgagtttttttcgttgttctgattgttgtttttatgaatttgcagatgcagatctttgtgaaaactctcaccggaaagactatcaccctagaggtggaaagttctgatacaatcgacaacgttaaggctaagatccaggataaggaaggaattcccccggatcagcaaaggcttatcttcgccggaaagcagttggaggacggacgtactctagctgattacaacatccagaaggagtctaccctccatttggtgctccgtctacgtggaggtatggcggctgctgcctcaccatctccatgcttctccaaaaccctacctccatcttcctccaaatcttccaccattcttcctagatctaccttccctttccacaatcaccctcaaa